(19) United States
(12) Patent Application Publication
(10) Pub. No.: US 2011/0154516 A1
(43) Pub. Date: Jun. 23, 2011

(54) HUMAN FACTOR IX VARIANTS WITH AN EXTENDED HALF LIFE

(76) Inventors: Darrel W. Stafford, Carrboro, NC (US); David M. Mann, Gaithersburg, MD (US); Dengmin Feng, Carrboro, NC (US)

(21) Appl. No.: 12/734,181

(22) PCT Filed: Oct. 15, 2008
(86) PCT No.: PCT/US2008/011754

§ 371 Date: Aug. 23, 2010

Related U.S. Application Data
(60) Provisional application No. 60/999,035, filed on Oct. 15, 2007.

(51) Int. Cl. C12N 9/50; C12N 15/57; A01K 67/00

(52) U.S. Cl. 800/13; 435/219; 536/23.2; 435/325

(57) ABSTRACT

Factor IX variants are described with an increase in the number of glycosylation sites. The Factor IX variants have an increased half life and/or recovery.

### PRIORITY STATEMENT

This application claims the benefit, under 35 U.S.C. §119(e), of U.S. Provisional Application Ser. No. 60/999,035, filed Oct. 15, 2007, the entire contents of which are incorporated by reference herein in their entirety.

### BACKGROUND OF THE INVENTION

1. Field of the Invention

The invention pertains to Factor IX variants containing additional glycosylation sites, as well as nucleic acid constructs encoding the Factor IX variants.

2. Description of the Related Art

Factor IX is commercially available as both a plasma-derived product (Mononine®) and a recombinant protein (Benefix®). Mononine® has the disadvantage that there is a potential to transmit disease through contamination with bacteria and viruses (such as HIV, hepatitis) which are carried through the purification procedure. The use of recombinant protein (e.g., Benefix®) avoids these problems. However, the pharmacokinetic properties of recombinant Factor IX (rFactor IX, e.g., Benefix®) do not compare well with the properties of human plasma-derived Factor IX (pdFactor IX, e.g., Mononine®) after intravenous (i.v.) bolus infusion in laboratory animal model systems and in humans. Due to the less favorable pharmacokinetic properties of rFactor IX, generally 20-30% higher doses of rFactor IX are required to achieve the same procoagulant activity level as pdFactor IX (White et al. (April 1998) Seminars in Hematology vol. 35, no. 2 Suppl. 2: 33-38; Roth et al. (Dec. 15, 2001) Blood vol. 98 (13): 3600-3606).

The addition of glycosylation sites to proteins has proved to be an important tool for extending their half life. For example, darbepoetin-α is a recombinant form of erythropoietin in which two additional N-linked glycosylation sites were added (Elliott et al. “Enhancement of therapeutic protein in vivo activities through glycoengineering” Nat. Biotechnol. (2003) 21:414-421). To create darbepoetin, residues 30 and 32 were mutated to create one glycosylation site and residues 87, 88 and 90 were mutated to create the second glycosylation site. Darbepoetin with these two additional glycosylation sites had a half life three times that of normal erythropoietin; moreover, its safety was indistinguishable from EPO. No cases of antibody development against darbepoetin have been identified as of 2004 even though the molecule has five amino acid changes (Smalling et al. “Drug-induced and antibody-mediated pure red cell aplasia: a review of literature and current knowledge” Biotechnol Annu Rev. (2004) 10:237-250; Sinclair et al. “Glycoengineering: the effect of glycosylation on the properties of therapeutic protein”. J Pharm Sci. (2005) 94:1626-1635). Adding neo-glycosylation sites also extended the half life of leptin and Mpl ligand.

The present invention relates to the production of Factor IX (FIX) variants having additional glycosylation sites. The recombinant Factor IX variants have greater recovery values and/or an increased half life so that lower dosages and/or less frequent doses of Factor IX may be administered to a subject.

### SUMMARY OF THE INVENTION

The present invention provides an isolated Factor IX (FIX) protein variant comprising one or more than one additional glycosylation site as compared to wild type Factor IX. The one or more additional glycosylation sites can be introduced by insertion of additional amino acids, deletion of amino acids, substitution of amino acids and/or rearrangement of amino acids, in any combination. The one or more additional glycosylation sites can also be introduced by site-directed mutagenesis and/or by chemical synthesis of the FIX variant.

In some embodiments, at least one of the additional glycosylation sites is in the activation peptide. The FIX variant can comprise a peptide segment inserted between position N157 and N167 of the human FIX amino acid sequence of SEQ ID NO:33 and the peptide segment can comprise from about 3 to about 100 amino acid residues. The peptide segment can comprise at least part of a mouse Factor IX activation peptide (e.g., FIG. 1, line 4) and the mouse activation peptide can be modified to increase the number of glycosylation sites (e.g., FIG. 1, lines 2 and 3). The FIX protein variant of this invention can be a human FIX protein.

The one or more than one additional glycosylation sites of the variant FIX of this invention can be N-linked glycosylation site(s), O-linked glycosylation site(s) and a combination of N-linked glycosylation site(s) and O-linked glycosylation site(s).

In some embodiments, glycosylation site(s) can comprise N-linked glycosylation site(s) comprising a consensus sequence NXT/S, with the proviso that X is not proline. In other embodiments, the glycosylation site(s) comprise O-linked glycosylation site(s) comprising a consensus sequence selected from the group consisting of CXXGGT/S-C (SEQ ID NO:9), NSTE/DA (SEQ ID NO:10), NITQS (SEQ ID NO:11), QSTQS (SEQ ID NO:12), D/E-FT-R/K-V (SEQ ID NO:13), C-E/D-SN (SEQ ID NO:14), GGSC-K/R (SEQ ID NO:15) and any combination thereof. Furthermore, the FIX variant of this invention can comprise about one to about five additional glycosylation sites.

The present invention further provides a vector comprising a nucleotide sequence encoding the FIX variant of this invention, a transformed cell comprising the vector of this invention and a transgenic animal comprising the FIX variant of this invention.

In some embodiments, at least one additional glycosylation site of the FIX variant of this invention, can be outside of the activation peptide.

Furthermore, the at least one additional glycosylation site of the FIX variant of this invention can correspond to a site that is glycosylated in the native form of a non-human homolog of FIX, which non-human homolog can be, e.g., dog, pig, cow or mouse.

Additionally provided herein is a method of increasing the number of glycosylation sites in a Factor IX protein comprising: a) aligning a first FIX amino acid sequence and a second FIX amino acid sequence; b) identifying a glycosylation site in the first FIX amino acid sequence that is not present in the second FIX amino acid sequence; c) modifying the second FIX amino acid sequence to introduce a glycosylation site corresponding to the glycosylation site identified in the first FIX amino acid sequence of step (b), wherein modifying the second FIX amino acid sequence increases the number of glycosylation sites in the FIX protein.

In the methods of this invention, the first FIX amino acid sequence can be from a non-human species and the second FIX amino acid sequence can be human FIX. In further embodiments of these methods, the glycosylation site in the first FIX amino acid sequence can be in the activation peptide or outside of the activation peptide. The methods further encompass the addition of one or more glycosylation site both in the activation peptide and outside the activation peptide.

The present invention further provides an isolated FIX variant comprising one or more additional sugar chains as compared to wild type FIX. In some embodiments, the one or more additional sugar chains are added to the FIX protein by chemical and/or enzymatic methods.

Further aspects, features and advantages of this invention will become apparent from the drawings described below.

### BRIEF DESCRIPTION OF THE DRAWINGS

These and other features of this invention will now be described with reference to the following figures, which are intended to illustrate and not to limit the invention.

FIG. 1 shows human Factor IX variants. Line 1: Human Factor IX (SEQ ID NO:5); Line 2: Human FIX with mouse active peptide (AP) segment without modification of the mouse AP segment (SEQ ID NO:2); Line 3: Human FIX with mouse AP with one glycosylation site added (SEQ ID NO:3); Line 4: Human FIX with mouse AP and two glycosylation sites added (SEQ ID NO:4). The small arrow denotes the first amino acid of mature FIX (SEQ ID NO:33). The two larger arrows denote the activation peptide cleavage sites. The black stars indicate two existing glycosylation sites in the human FIX protein. The grey stars denote proposed additional glycosylation sites.

FIG. 2 shows an alignment of the amino acid sequence of human Factor IX (SEQ ID NO:5) with homologous amino acid sequences from dog (SEQ ID NO:16), pig (SEQ ID NO:17), cow (SEQ ID NO:18), and mouse (SEQ ID NO:19) (Lines 2, 3, 4 and 5, respectively). The two arrows denote the activation peptide cleavage sites. The stars indicate existing glycosylation sites in at least one of the five species shown.

FIG. 3 shows alignment of the activation peptides of several mammalian species (bovine (SEQ ID NO:20), sheep (SEQ ID NO:21), horse (SEQ ID NO:22), dog (SEQ ID NO:23), cat (SEQ ID NO:24), rat (SEQ ID NO:25), mouse (SEQ ID NO:26), human (SEQ ID NO:27), pig (SEQ ID NO:28), rabbit (SEQ ID NOS:29 & 30), and guinea pig (SEQ ID NO:31). The highly variable region is displayed as black background with white lettering. The consensus sequence shown corresponds to SEQ ID NO:32.

FIG. 4 shows a box plot of the half life of the human FIX variant containing one extra glycosylation site compared to wild-type recombinant human factor IX. The half-life of this FIX variant is increased by about 1.5 hour. Each of the box plot results represent half life determination for eight mice; the median for each box plot is represented by the solid horizontal line and the extremes of each set of mice are shown by the error bars on the graph.

FIG. 5 shows the alignment of the complete FIX amino acid sequence for cow, dog, human, mouse, platypus and opossum.

FIG. 6 shows 168 additional examples of FIX variants of this invention, wherein O-linked glycosylation site attachment sequences have been inserted into regions outside the activation peptide.

Further aspects, features and advantages of this invention will become apparent from the detailed description of the embodiments which follow.

### DETAILED DESCRIPTION OF THE INVENTION

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

### Definitions

As used herein, “a,” “an” or “the” can mean one or more than one. For example, “a” cell can mean a single cell or a multiplicity of cells.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount (e.g., an amount of methylation) and the like, is meant to encompass variations of ±20%, ±10%, ±5%, ±1%, ±0.5%, or even ±0.1% of the specified amount.

As used herein, the transitional phrase “consisting essentially of” means that the scope of a claim is to be interpreted to encompass the specified materials or steps recited in the claim, “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP §2111.03. Thus, the term “consisting essentially of” when used in a claim of this invention is not intended to be interpreted to be equivalent to “comprising.”

The term “pharmacokinetic properties” has its usual and customary meaning and refers to the absorption, distribution, metabolism and excretion of the Factor IX protein.

The usual and customary meaning of “bioavailability” is the fraction or amount of an administered dose of a biologically active drug that reaches the systemic circulation. In the context of embodiments of the present invention, the term “bioavailability” includes the usual and customary meaning but, in addition, is taken to have a broader meaning to include the extent to which the Factor IX protein is bioactive. In the case of Factor IX, for example, one measurement of “bioavailability” is the procoagulant activity of Factor IX protein obtained in the circulation post-infusion.

“Posttranslational modification” has its usual and customary meaning and includes but is not limited to removal of leader sequence, γ-carboxylation of glutamic acid residues, β-hydroxylation of aspartic acid residues, N-linked glycosylation of asparagine residues, O-linked glycosylation of serine and/or threonine residues, sulfation of tyrosine residues, phosphorylation of serine residues and any combination thereof.

As used herein, “biological activity” is determined with reference to a standard derived from human plasma. For Factor IX, the standard is MONONINE® (ZLB Behring). The biological activity of the standard is taken to be 100%.

The term “processing factor” is a broad term which includes any protein, peptide, non-peptide cofactor, substrate and/or nucleic acid which promotes the formation of a functional Factor IX. Examples of such processing factors include, but are not limited to, paired basic amino acid converting (or cleaving) enzyme (PACE), Vitamin K epoxide reductase (VKOR), and Vitamin K dependent γ-glutamyl carboxylase (VKGC).

The term “Factor IX protein” as used herein includes wild type Factor IX protein as well as naturally occurring or man-made variants (e.g., the T/A dimorphism in the activation peptide of human FIX at position 148 (numbering based on the mature human FIX amino acid sequence of SEQ ID NO:33, which shows a T at position 148) as described in Graham et al. (“The Malmo polymorphism of coagulation factor IX, an immunologic polymorphism due to dimorphism of residue 148 that is in linkage disequilibrium with two other F.IX polymorphisms” Am. J. Hum. Genet. 42:573-580 (1988)) Thus, a FIX protein of this invention includes a mature human FIX protein having the amino acid sequence of SEQ ID NO:33, wherein the amino acid at position 148 can be a T or an A and a subject can be either heterozygous or homozygous for either T or A at this site. A FIX protein of this invention can further include mutated forms of FIX as are known in the literature (see, e.g., Chang et al. “Changing residue 338 in human factor IX from arginine to alanine causes an increase in catalytic activity” J. Biol. Chem. 273:12089-94 (1998); Cheung et al. “Identification of the endothelial cell binding site for factor IX” PNAS USA 93:11068-73 (1996); Horst, Molecular Pathology, page 361 (458 pages) CRC Press, 1991, the entire contents of each of which are incorporated by reference herein). A FIX protein of this invention further includes any other naturally occurring human FIX variant or man made human FIX variant now known or later identified, derivatives and active fragments/active domains thereof, as are known in the art. A Factor IX protein of this invention further includes the pharmacologically active form of FIX, which is the molecule from which the activation peptide has been cleaved out of the protein by the action of proteases (or by engineering it out of the protein by removing it at the nucleic acid level), resulting in two non-contiguous polypeptide chains for FIX (light chain and heavy chain) folded as the functional FIX clotting factor. Specifically, Factor IX variants having a modification to increase the degree of glycosylation (e.g., N-linked and/or O-linked glycosylation) are specifically included in the broad term.

The term “half life” is a broad term which includes the usual and customary meaning as well as the usual and customary meaning found in the scientific literature for Factor IX. Specifically included in this definition is a measurement of a parameter associated with Factor IX which defines the time post-infusion for a decrease from an initial value measured at infusion to half the initial value. In some embodiments, the half life of FIX can be measured in blood and/or blood components using an antibody to Factor IX in a variety of immunoassays, as are well known in the art and as described herein. Alternatively, half life may be measured as a decrease in Factor IX activity using functional assays including standard clotting assays, as are well known in the art and as described herein.

The term “recovery” as used herein includes the amount of FIX, as measured by any acceptable method including but not limited to FIX antigen levels or FIX protease- or clotting-activity levels, detected in the circulation of a recipient animal or human subject at the earliest practical time of removing a biological sample (e.g., a blood or blood product sample) for the purpose of measuring the level of FIX following its infusion, injection, or delivery or administration otherwise. With current methodologies, the earliest biological sampling time for measuring FIX recovery typically falls within the first 15 minutes post infusion, injection, or delivery/administration otherwise of the FIX, but it is reasonable to expect quicker sampling times as scientific and/or clinical technologies improve. In essence, the recovery value for FIX is meant here to represent the maximum fraction of infused, injected or otherwise delivered/administered FIX that can be measured in the circulation of the recipient at the earliest possible time point following infusion, injection, or otherwise delivery to a recipient animal or patient.

The term “glycosylation site(s)” is a broad term that has its usual and customary meaning. In the context of the present application the term applies to both sites that potentially could accept a carbohydrate moiety, as well as sites within the protein, specifically FIX, on which a carbohydrate moiety has actually been attached and includes any amino acid sequence that could act as an acceptor for oligosaccharide and/or carbohydrate.

The term “isolated” can refer to a nucleic acid or polypeptide that is substantially free of cellular material, viral material, and/or culture medium (when produced by recombinant DNA techniques), or chemical precursors or other chemicals (when chemically synthesized). Moreover, an “isolated fragment” is a fragment of a nucleic acid or polypeptide that is not naturally occurring as a fragment and would not be found in the natural state.

An “isolated cell” refers to a cell that is separated from other cells and/or tissue components with which it is normally associated in its natural state. For example, an isolated cell is a cell that is part of a cell culture. An isolated cell can also be a cell that is administered to or introduced into a subject, e.g., to impart a therapeutic or otherwise beneficial effect.

Some embodiments of the invention are directed to Factor IX variants having one or more additional glycosylation sites. By “additional” or “new” glycosylation sites is meant that the number of glycosylation sites in the FIX variant is greater than the number of glycosylation sites normally present in a “wild type” form of Factor IX. A Factor IX protein of this invention can include plasma derived FIX as well as recombinant forms of FIX. Generally, embodiments of the invention are directed to increasing the number of glycosylation sites in a FIX molecule of this invention. However, it is to be understood that a Factor IX protein of this invention that can be modified to increase the number of glycosylation sites and/or to increase the number of sugar chains is not limited to a particular “wild type” FIX amino acid sequence because naturally occurring or man-made FIX variants can also be modified according to the methods of this invention to increase the number of glycosylation sites and/or to increase the number of sugar chains.

The present invention is further directed to FIX variants containing additional sugar chains. Such additional sugar side chains can be present at one or more of the additional glycosylation sites introduced into the FIX variants of this invention by the methods described herein. Alternatively, the additional sugar side chains can be present at sites on the FIX protein as a result of chemical and/or enzymatic methods to introduce such sugar chains to the FIX molecule, as are well known in the art. By “additional” or “new” sugar chains is meant that the number of sugar chains in the FIX variant is greater than the number of sugar chains normally present in a “wild type” form of Factor IX. In various embodiments, about 1 to about 500 additional sugar side chains (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) can be added.

In some embodiments, at least one additional glycosylation site is in the activation peptide of Factor IX (e.g., the human FIX activation peptide having the amino acid sequence of SEQ ID NO:1). In particular embodiments, the FIX variant has an insertion of a peptide segment that introduces one or more glycosylation sites between position N157 and N167 of the human Factor IX amino acid sequence of SEQ ID NO:33.

Insertion(s) can be introduced into a FIX variant of this invention to increase the number of glycosylation sites and such insertion(s) can include from about one to about 100 amino acid residues, including any number of amino acid residues from one to 100 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and 100).

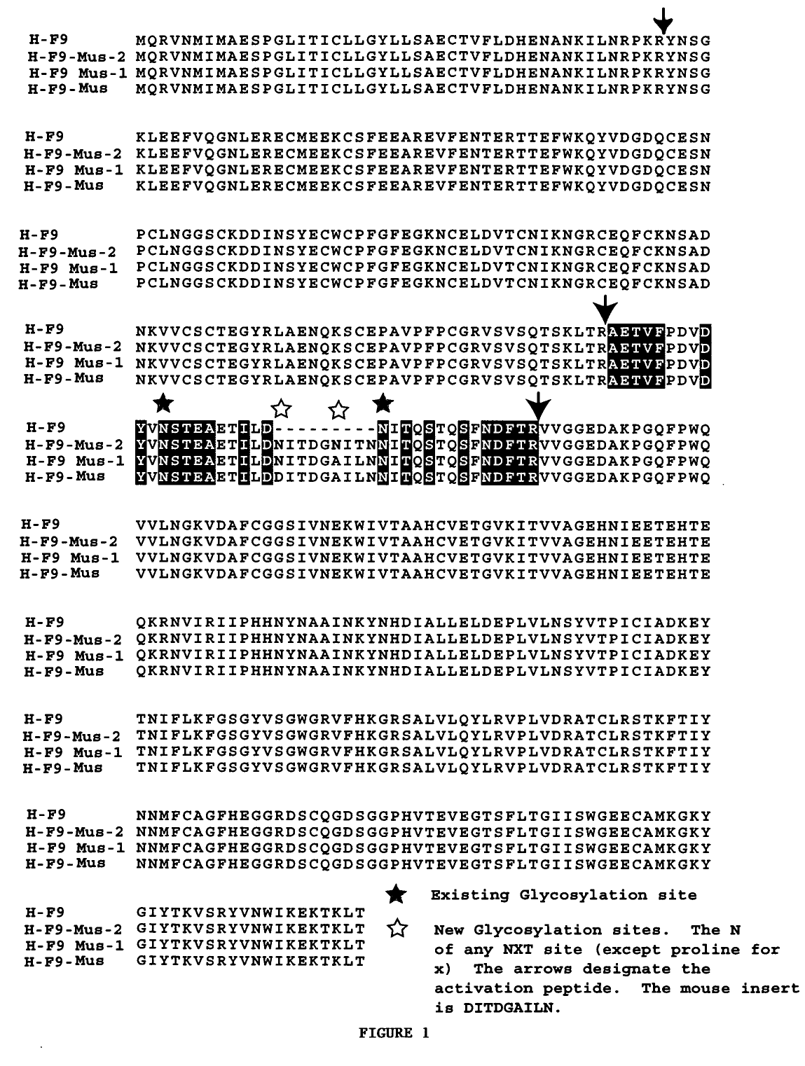

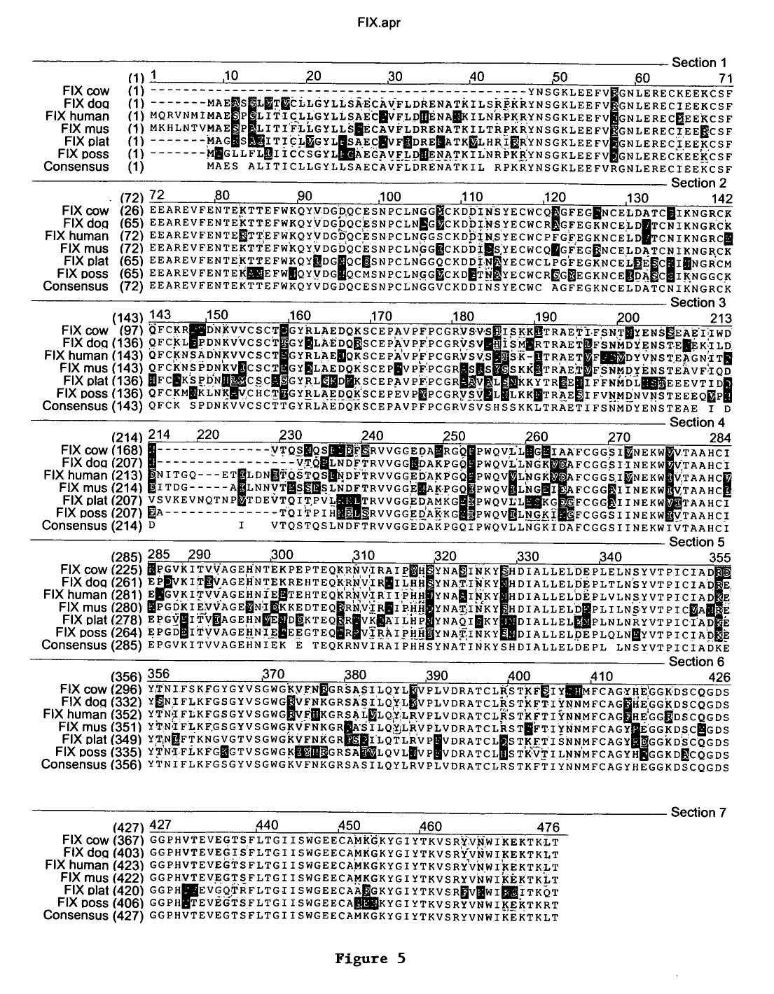

In particular embodiments, the insertion includes all or at least part (e.g., at least 3, 4, 5, 6, 7, 8, 9, 10, 11, 12, 13, 14, 15 or more amino acid residues) of a Factor IX activation peptide from a non-human species, such as mouse (as shown, e.g., in line 4 of FIG. 1 and in SEQ ID NO:2). In other embodiments, the human FIX sequence is modified to include the non-human (e.g., mouse) FIX activation peptide which is modified to increase the number of glycosylation sites (as shown, e.g., in lines 2 and 3 of FIG. 1 and in SEQ ID NOs:3 and 4). In further embodiments, the human FIX amino acid sequence can be modified by insertion of an amino acid segment of the activation peptide of any non-human FIX protein, including platypus (FIG. 5). SEQ ID NO:305 provides the 14 amino acid segment that can be introduced into the activation peptide of human FIX, e.g., between amino acid residues 166 and 167 as shown in FIG. 1 for the inserted peptide segment from the mouse activation peptide or at any other site in the activation peptide. SEQ ID NO:306 provides a mature human FIX variant with the 14 amino acid sequence of platypus inserted between amino acid residues 166 and 167. This inserted peptide sequence can be further modified to introduce additional glycosylation sites according to the teachings herein. The amino acid sequence for platypus FIX as provided in FIG. 5 also indicates that at least 14 amino acids can be inserted into the activation peptide of a FIX protein with an expectation that the activity and/or function of the protein would not be adversely affected.

The glycosylation site(s) may be selected from N-linked glycosylation site(s), O-linked glycosylation site(s) and/or a combination of N-linked glycosylation site(s) and O-linked glycosylation site(s). In some embodiments, the added glycosylation site(s) include N-linked glycosylation site(s) and the consensus sequence is NXT/S, with the proviso that X is not proline.

In some embodiments about one to about 5 glycosylation site(s) can be added to the FIX amino acid sequence. In various embodiments, about 1 to about 50 glycosylation site(s) (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) can be added. Embodiments of the invention include FIX variants in which either an N-linked or O-linked glycosylation site has been created by insertion, deletion or substitution of specific amino acids. In particular embodiments, the insertion, deletion and/or substitution is in the region of the activation peptide shown by the arrows in FIG. 1. The amino acid sequence of the human FIX activation peptide is provided herein as SEQ ID NO:1.

In some embodiments, the added glycosylation site(s) include O-linked glycosylation site(s) and the consensus sequence can be but is not limited to CXXGGT/S-C (SEQ ID NO:9), NSTE/DA (SEQ ID NO:10), NITQS (SEQ ID NO:11), QSTQS (SEQ ID NO:12), D/E-FT-R/K-V (SEQ ID NO:13), C-E/D-SN (SEQ ID NO:14), and GGSC-K/R (SEQ ID NO:15).

It is contemplated that the additional glycosylation sites introduced into a FIX amino acid sequence can be introduced anywhere throughout the amino acid sequence of the FIX protein. Thus, in some embodiments, the additional glycosylation site or sites are introduced in the activation peptide (denoted by arrows in FIG. 1; amino acids 146-180 of the mature human FIX amino acid sequence of SEQ ID NO:33), outside the activation peptide (e.g., before and/or after the activation peptide) or both inside the activation peptide and outside the activation peptide. Thus, based on the numbering of the 415 amino acids of the amino acid sequence of the mature human FIX protein as shown in SEQ ID NO:33, a glycosylation attachment site can be introduced by inserting additional amino acid residues between any of amino acids 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415 and any combination thereof. As used herein, a “glycosylation attachment site” or “glycosylation site” can mean a sugar attachment consensus sequence (i.e., a series of amino acids that act as a consensus sequence for attaching a sugar (mono-, oligo-, or poly-saccharide) to an amino acid sequence or it can mean the actual amino acid residue to which the sugar moiety is covalently linked. The sugar moiety can be a monosaccharide (simple sugar molecule), on oligosaccharide, or a polysaccharide.

In particular embodiments, additional amino acids can be inserted between and/or substituted into any of the amino acid residues that make up the activation peptide, such as between any of amino acids 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182 and any combination thereof. Furthermore, the same insert of this invention can be introduced multiple times at the same and/or at different locations in the amino acid sequence of the FIX protein, including within the activation peptide. Also, different inserts and/or the same inserts can be introduced one or more times at the same and/or at different locations between amino acid residues throughout the amino acid sequence of the FIX protein, including within the activation peptide.

It is well known in the art that some proteins can support a large number of sugar side chains and the distance between O-linked glycosylation sites can be as few as every other amino acid (see, e.g., Kolset & Tveit “Serglycin—structure and biology” Cell. Mol. Life. Sci 65:1073-1085 (2008) and Kiani et al. “Structure and function of aggrecan” Cell Research 12(1):19-32 (2002)). For N-linked glycosylation sites, the distance between sites can be as few as three, four, five or six amino acids (see, e.g., Lundin et al. “Membrane topology of the Drosophila OR83b odorant receptor” FEBS Letters 581:5601-5604 (2007); Apweiler et al. “On the frequency of protein glycosylation, as deduced from analysis of the SWISS-PROT database” Biochimica et Biophysica Acta 1473:4-8 (19991), the entire contents of each of which are incorporated by reference herein).

Furthermore, the FIX protein of this invention can be modified by mutation (e.g., substitution, addition and/or deletion of amino acids) to introduce N-linked glycosylation sites, O-linked glycosylation sites or both N-linked and O-linked glycosylation sites. For example, amino acid residues on the surface of the functional FIX protein can be identified according to molecular modeling methods standard in the art that would be suitable for modification (e.g., mutation) to introduce one or more glycosylation sites. One particular example of this approach is provided in Table 2, which shows the results of molecular modeling calculations used to determine the relative surface accessibility of each amino acid in the mature human FIX protein. The solvent accessibility calculations are based on a crystallographic structure determination of the actual three dimensional structure of this FIX protein. The first column lists the amino acid name, the second column lists the sequence position for that corresponding amino acid and the column entitled “Total” shows the calculated solvent accessibility values, in relative units, for each amino acid. A higher value in the Total column means that particular amino acid is calculated to be far more exposed to the solvent (i.e., on the surface of the folded protein). For the present invention, a cut off value of greater than or equal to 60 was arbitrarily selected in order to identify amino acid residues on the surface of the FIX molecule that could be modified according to the methods of this invention to increase the number of glycosylation sites.

For example, in some embodiments, three consecutive amino acid residues having a Total value of greater than or equal to 60 can be considered for modification to introduce an additional glycosylation site and such regions are shaded in the Total column of Table 2. (The amino acid residues that make up the activation peptide are also shaded in Table 2.) However, 60 is an arbitrary cut off used as an example, and any other cut off value could be selected in order to select amino acid candidates for modification to incorporate an additional glycosylation site. Furthermore, this approach is merely one example of how amino acid residues in the FIX protein can be selected for modification and thus, the amino acid residues that can be modified to incorporate additional glycosylation sites into the mature human FIX protein are not limited to those having any particular value in the Total column of Table 2. It is within the scope of this invention and within the skill of one of ordinary skill in the art to modify any amino acid residue or residues in the mature FIX amino acid sequence according to methods well known in the art and as taught herein and to test any resulting FIX variant for activity, stability, recovery, half life, etc., according to well known methods and as described herein (see, e.g., Elliott et al. “Structural requirements for additional N-linked carbohydrate on recombinant human erythropoietin” J. Biol. Chem. 279:16854-62 (2004), the entire contents of which are incorporated by reference herein).

Embodiments of the invention are directed to recombinant Factor IX variants in which glycosylation sites have been added to improve the recovery and/or half-life and/or stability of Factor IX. The glycosylation sites may be N-linked and/or O-linked glycosylation sites. In specific embodiments, at least one N-linked glycosylation site is added. Numerous examples of human FIX variants with one or more additional N-linked glycosylation sites in the activation peptide are provided herein as SEQ ID NOs:34-91.

Numerous other examples of human FIX variants with one or more additional O-linked glycosylation sites in the activation peptide are provided herein as SEQ ID NOs:92-132. Furthermore, numerous examples of human FIX variants with one or more additional N-linked glycosylation site and one or more O-linked glycosylation site in the activation peptide are provided herein by combining the modifications made to introduce N-linked glycosylation sites as shown in SEQ ID NOs:34-91 with the modifications made to introduce the O-linked glycosylation sites as shown in SEQ ID NOs:92-132, in any combination and in any order. Such combinations can further comprise any additional modifications in the activation peptide and/or outside of the activation peptide that introduce even more glycosylation sites. Such combined modifications as described for the amino acid sequences set forth herein as SEQ ID NOs:34-132 are readily identifiable by one of ordinary skill in the art and are included among the embodiments of this invention to the same extent as if each individual amino acid sequence setting forth all such combinations were explicitly provided herein.

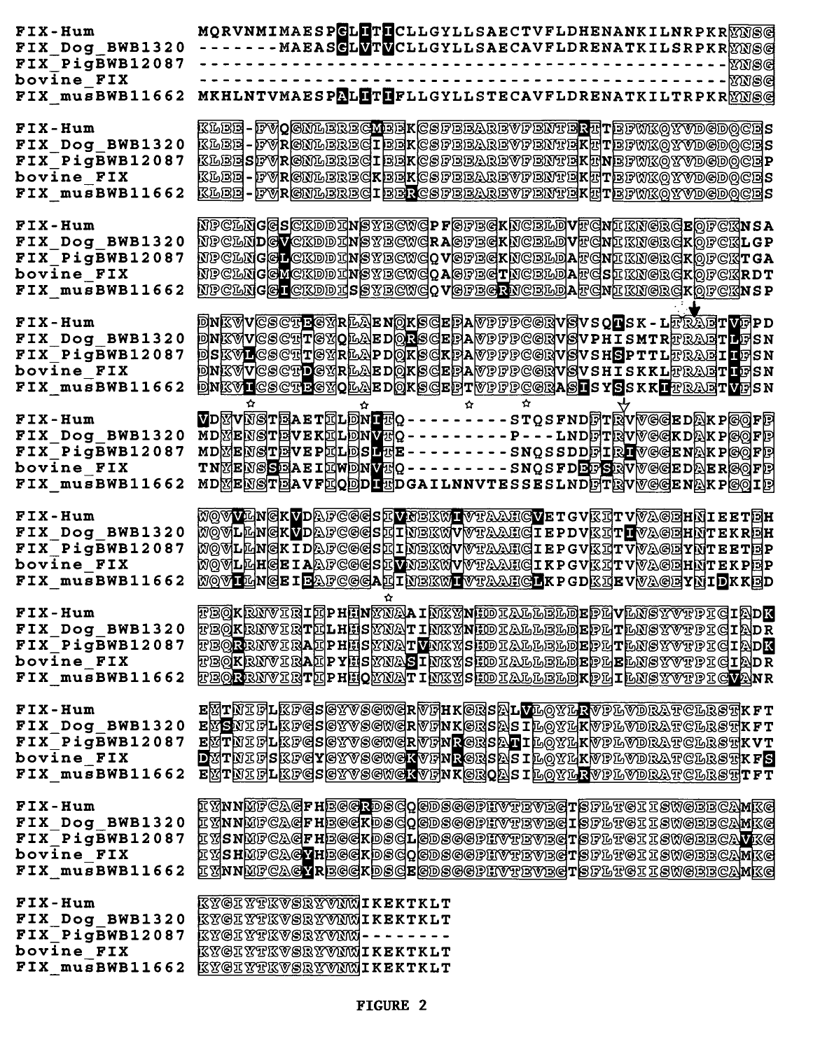

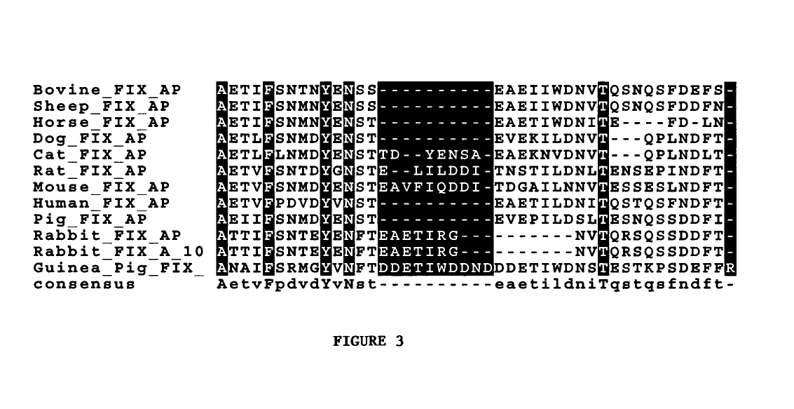

As noted herein, in some embodiments, at least one additional glycosylation site is introduced into the FIX amino acid sequence at a site that is outside of the activation peptide. Preferably, the at least one additional glycosylation site corresponds to a site that is glycosylated in the native form of a non-human homolog of Factor IX, as shown for example, in FIG. 2, wherein a glycosylation site is identified at amino acids 260-262 in all non-human species shown in the figure but is not naturally present in the human FIX protein. A modification of the human FIX amino acid sequence to introduce a serine or threonine at amino acid 262 of the amino acid sequence of SEQ ID NO:33, which is the mature (i.e., secreted) form of human FIX, would introduce an additional N-linked glycosylation site in the human protein. Preferably, the non-human homolog is from dog, pig, cow, or mouse.

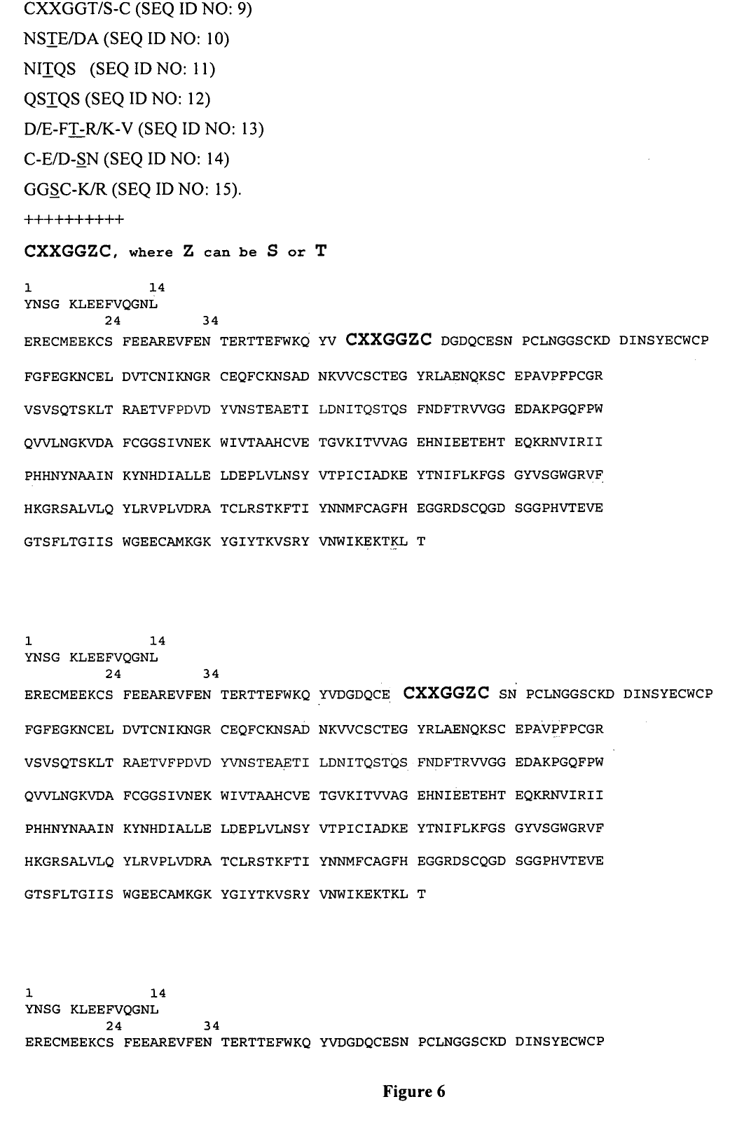

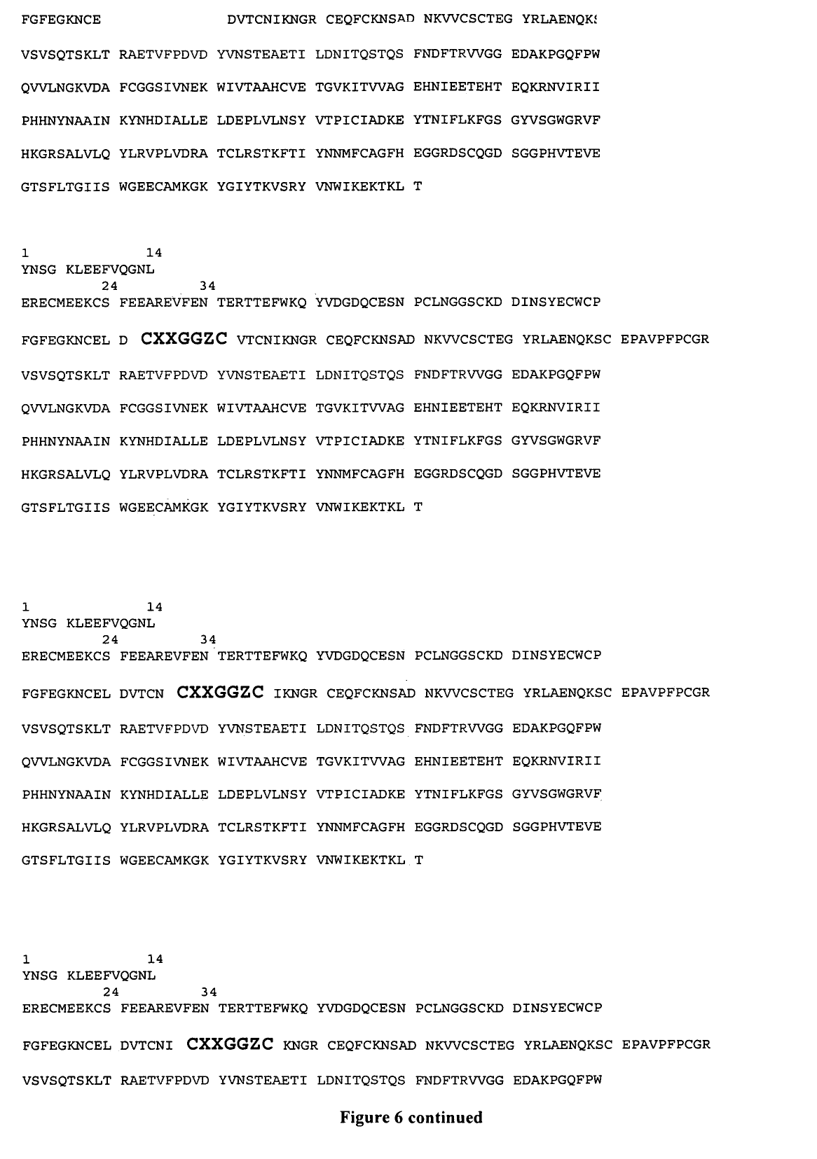

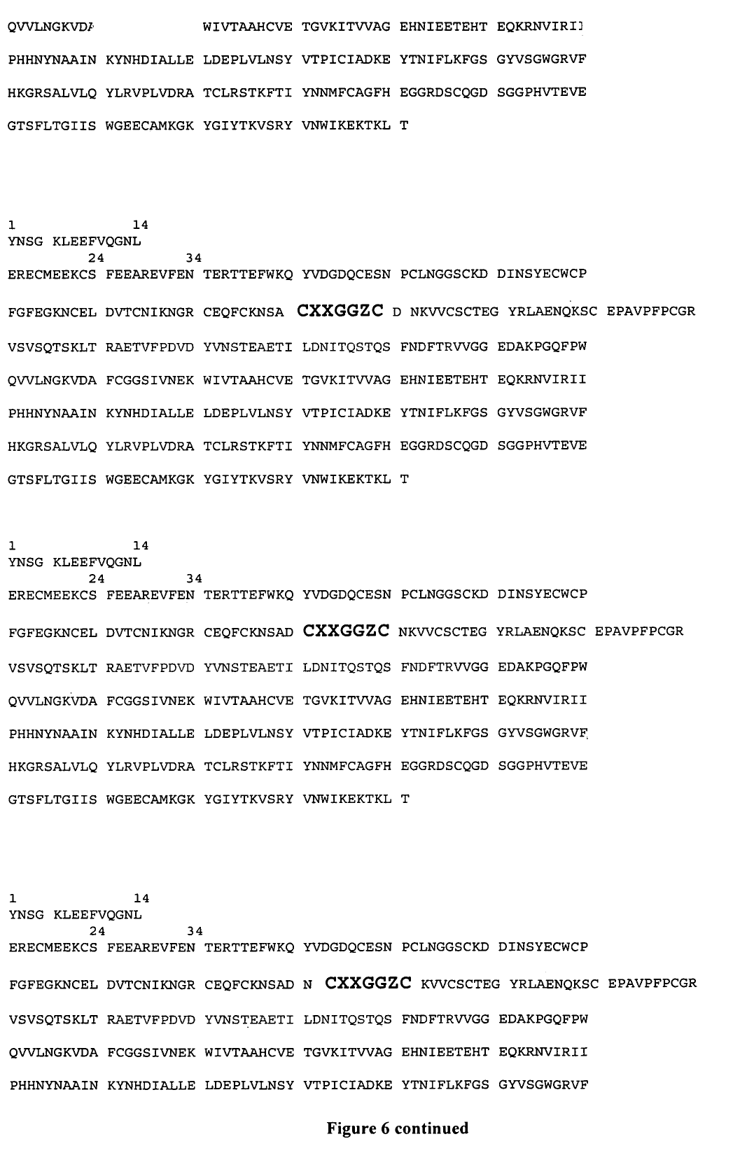

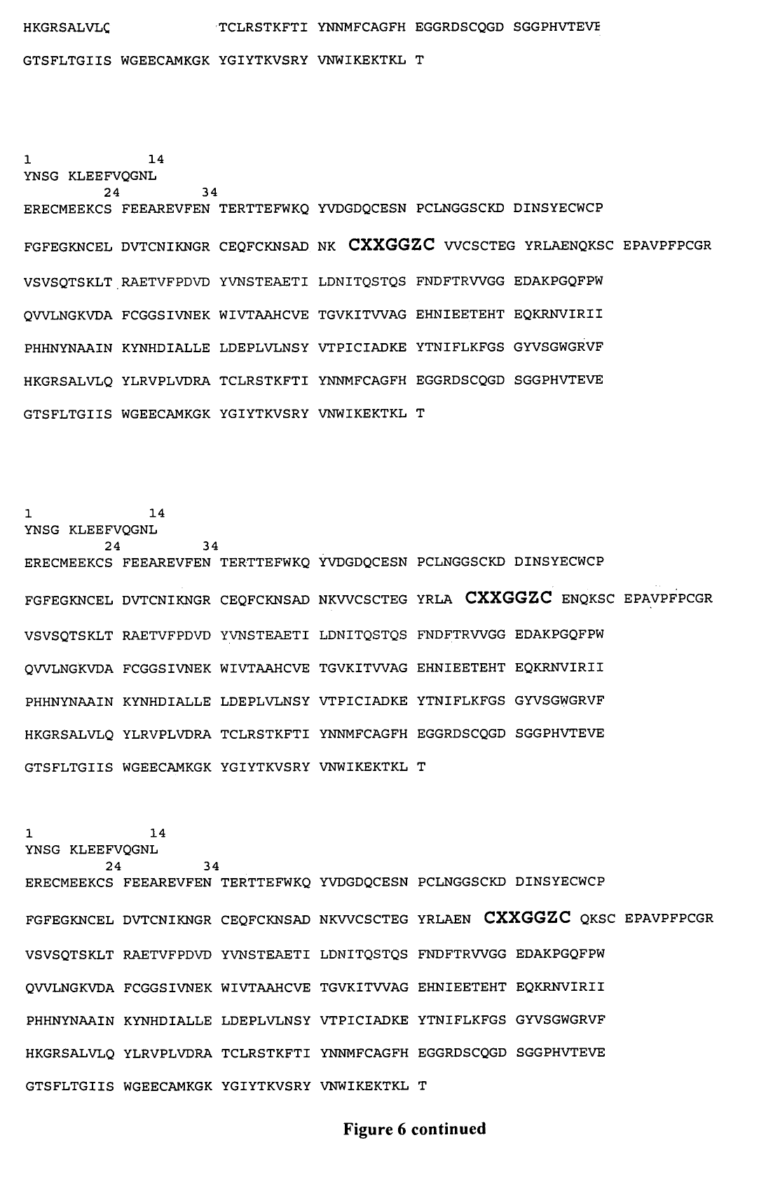

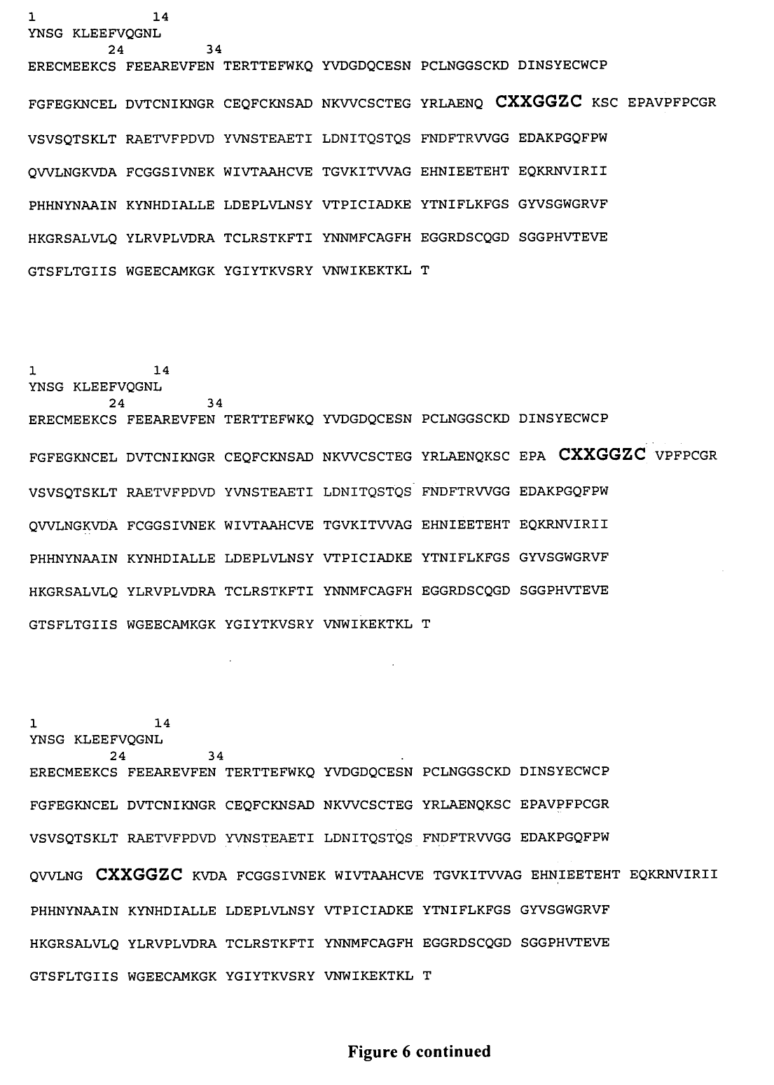

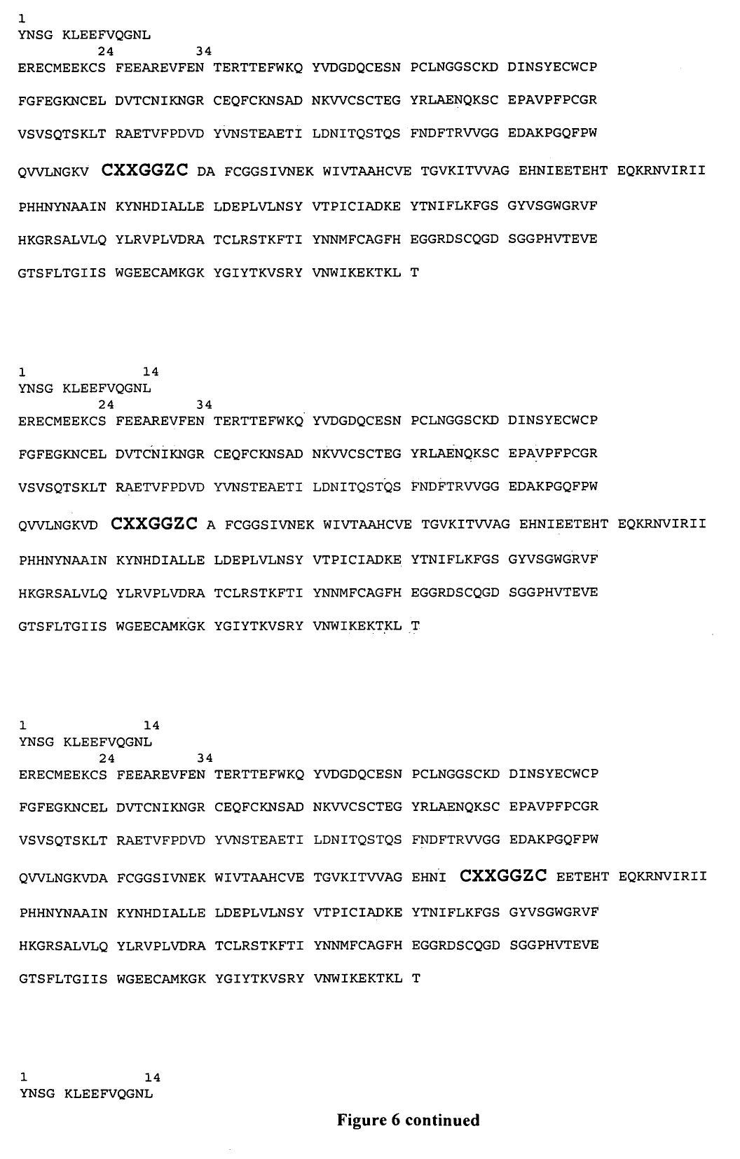

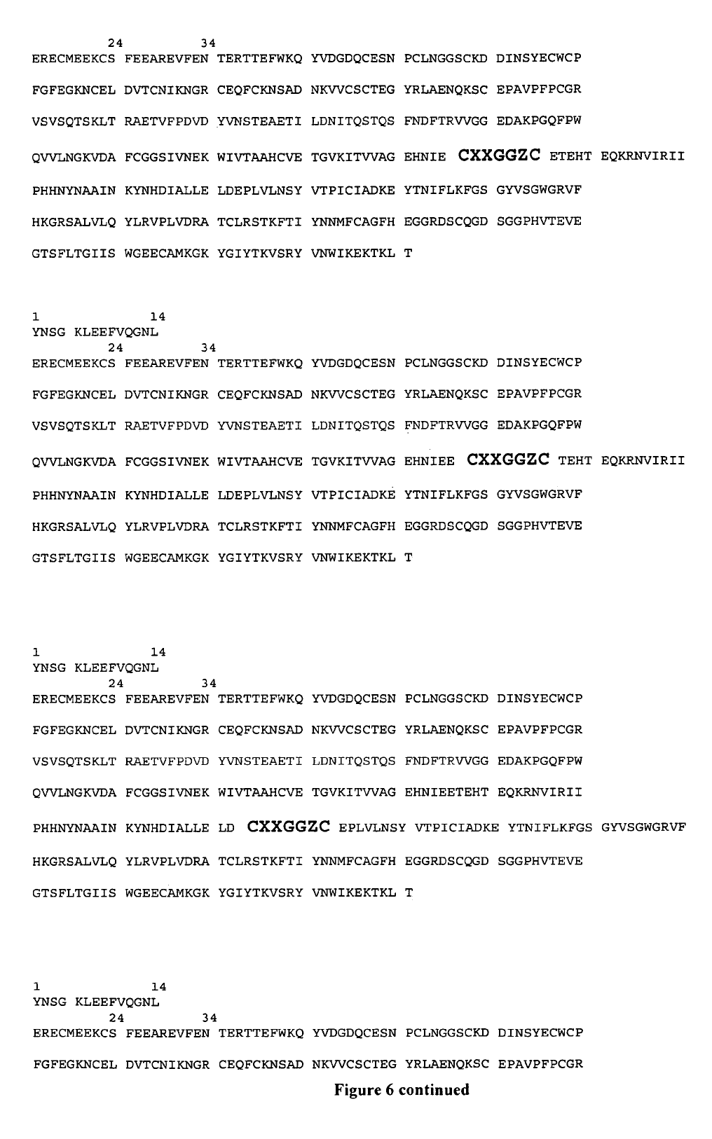

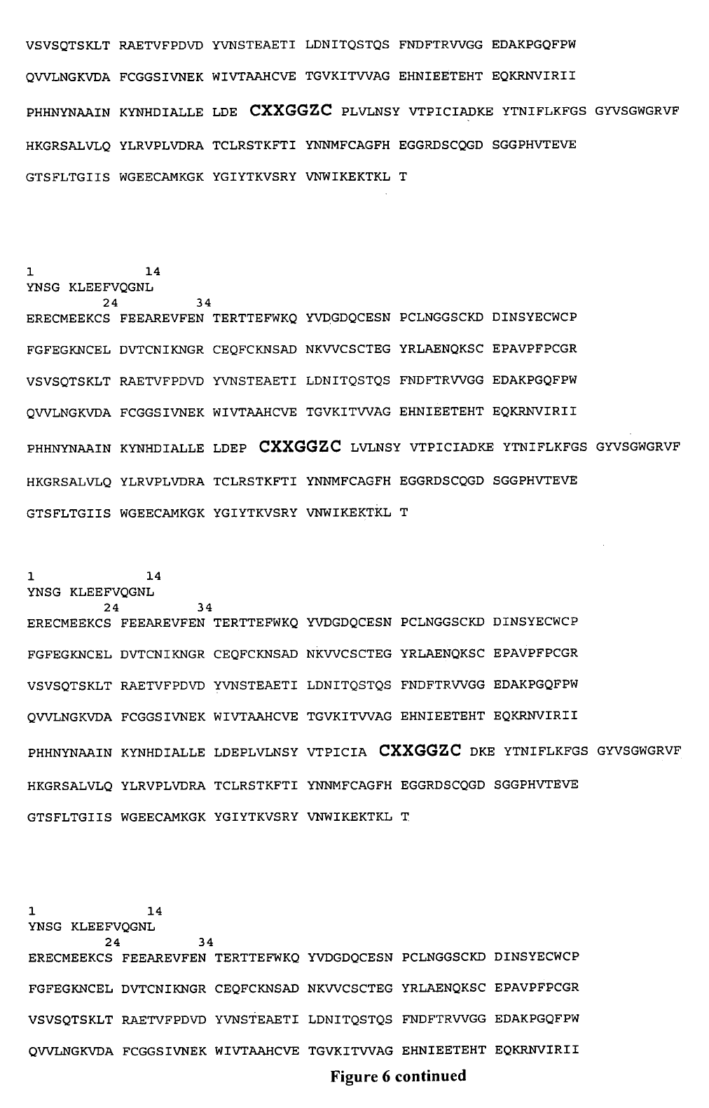

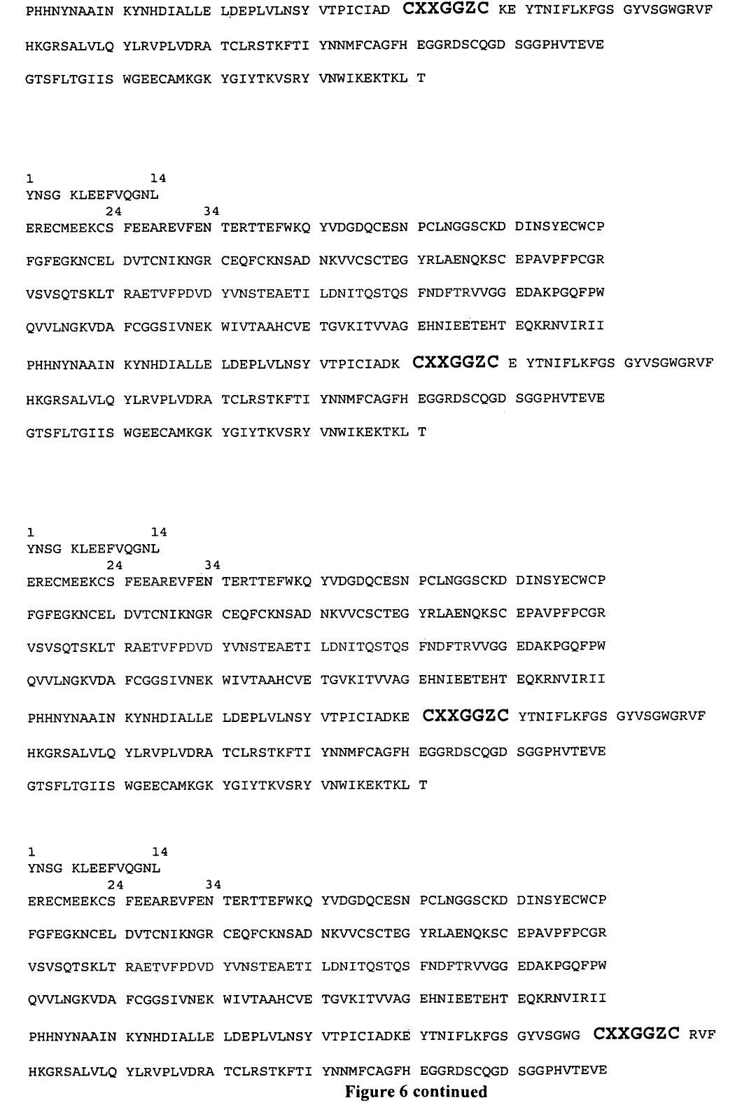

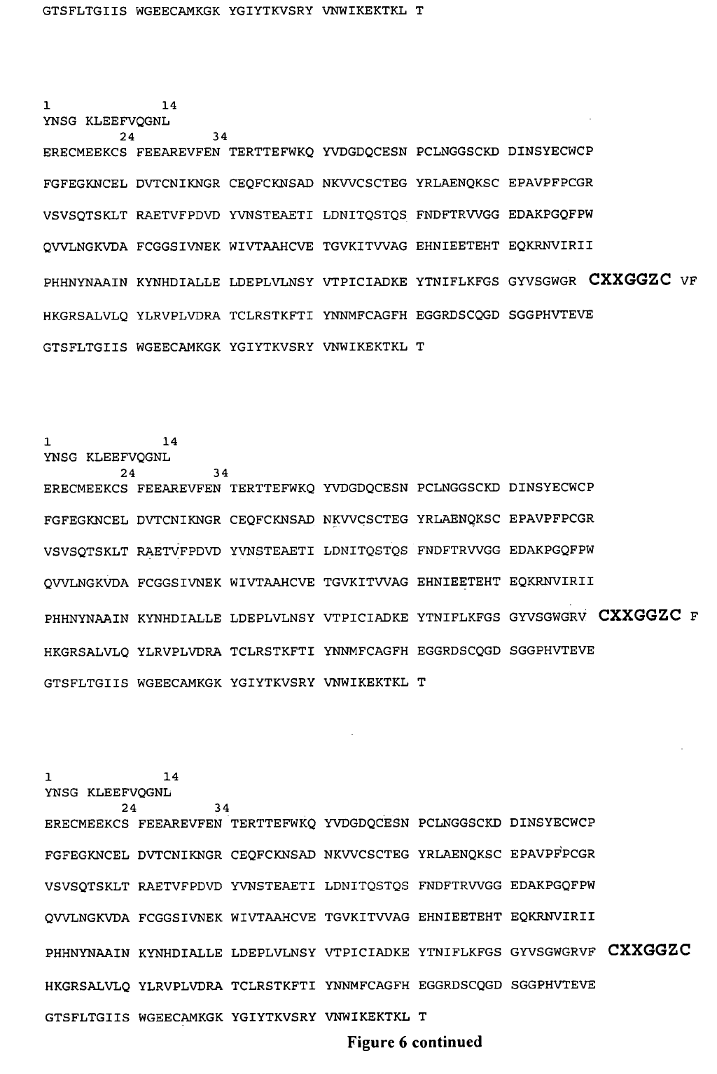

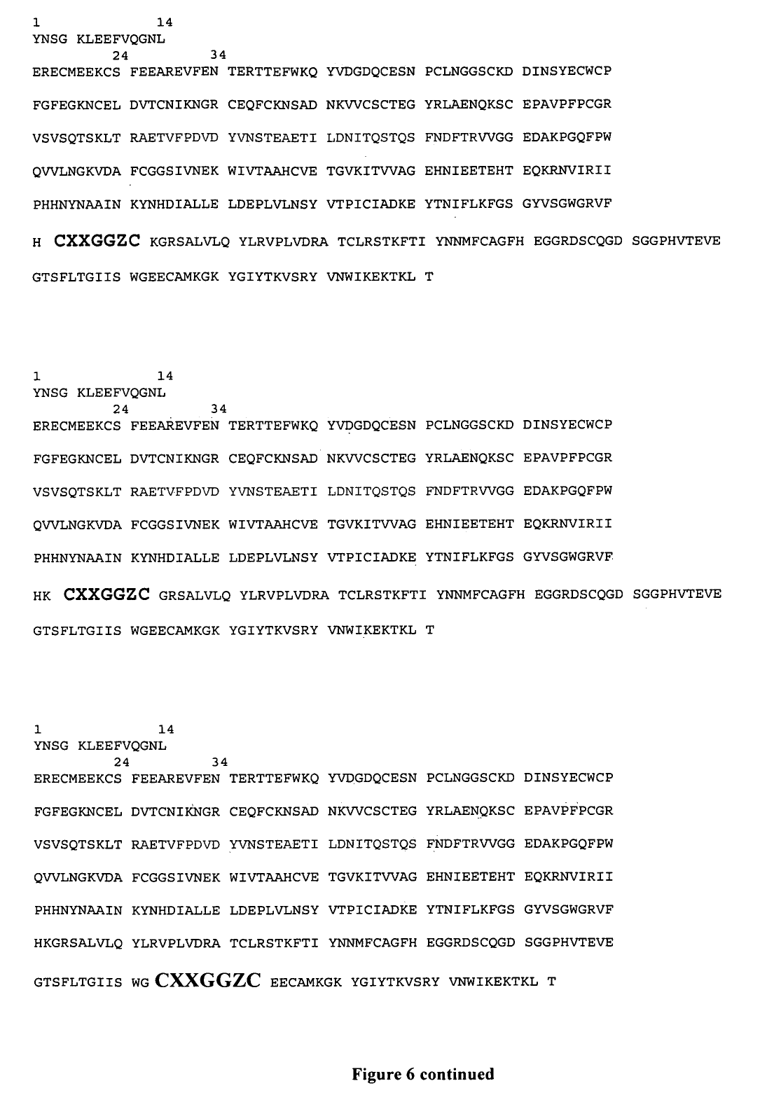

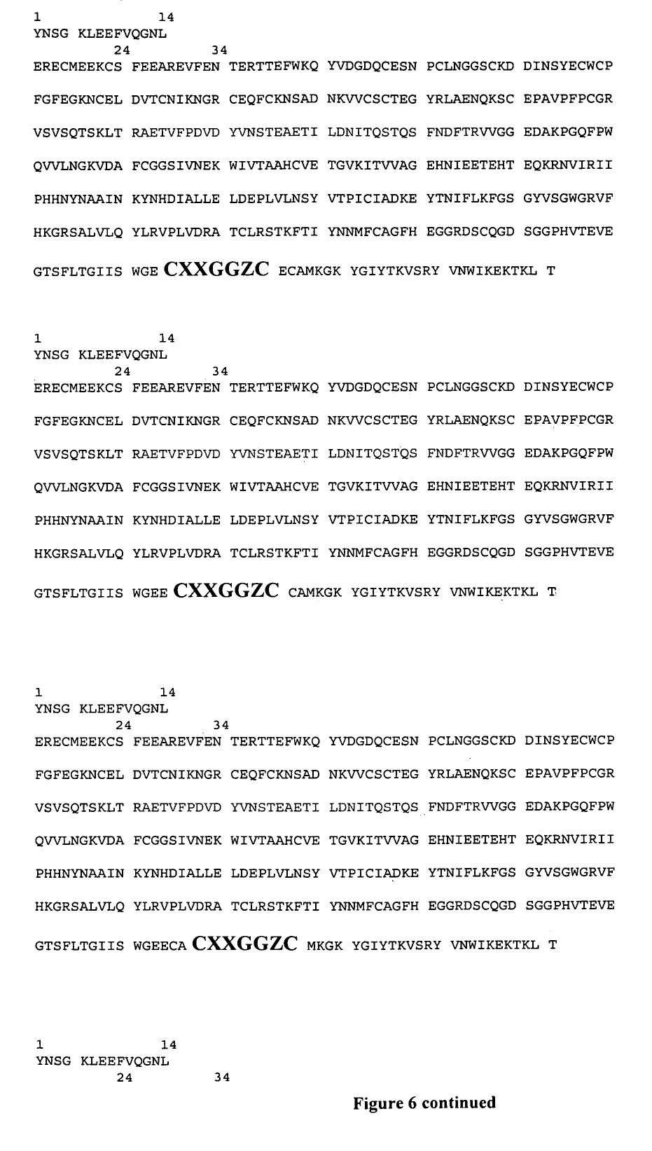

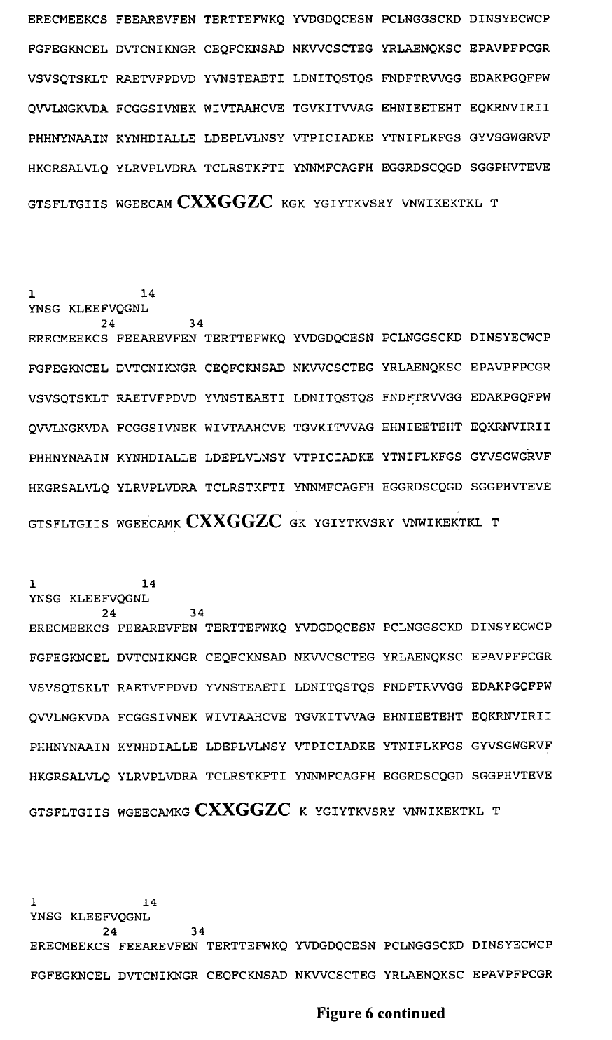

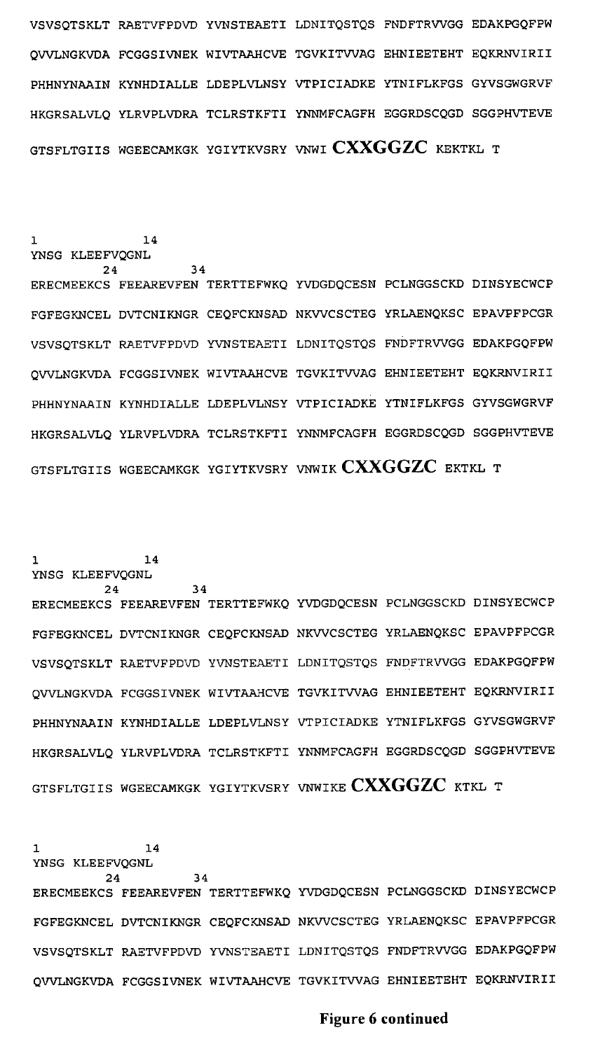

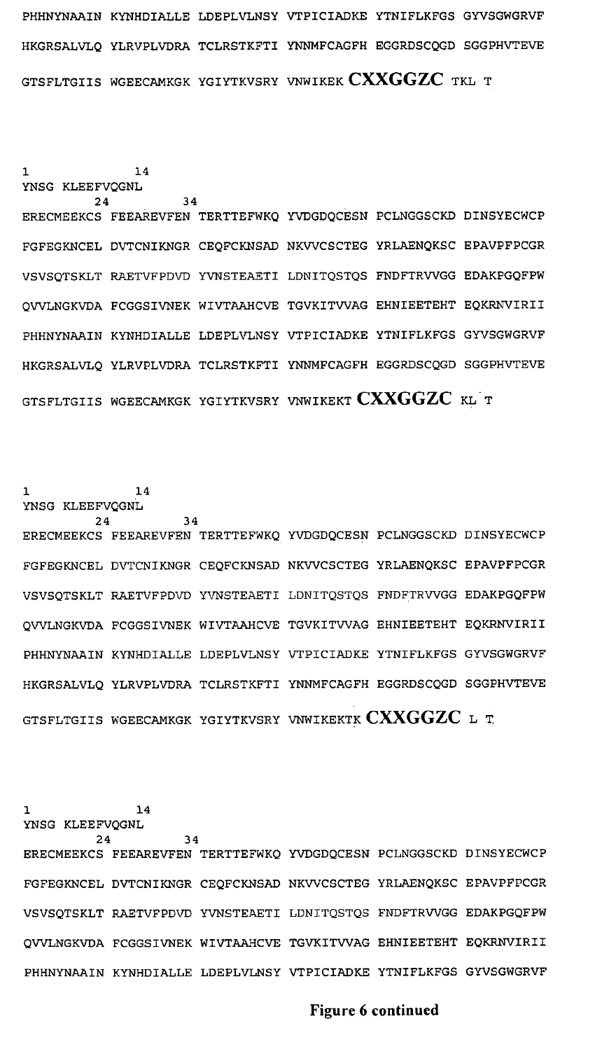

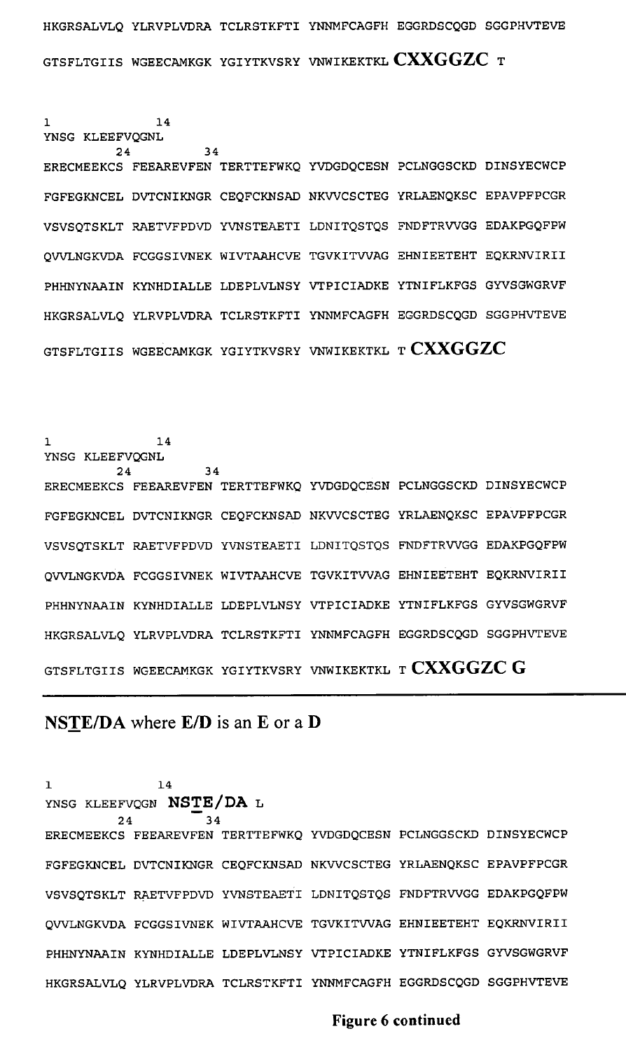

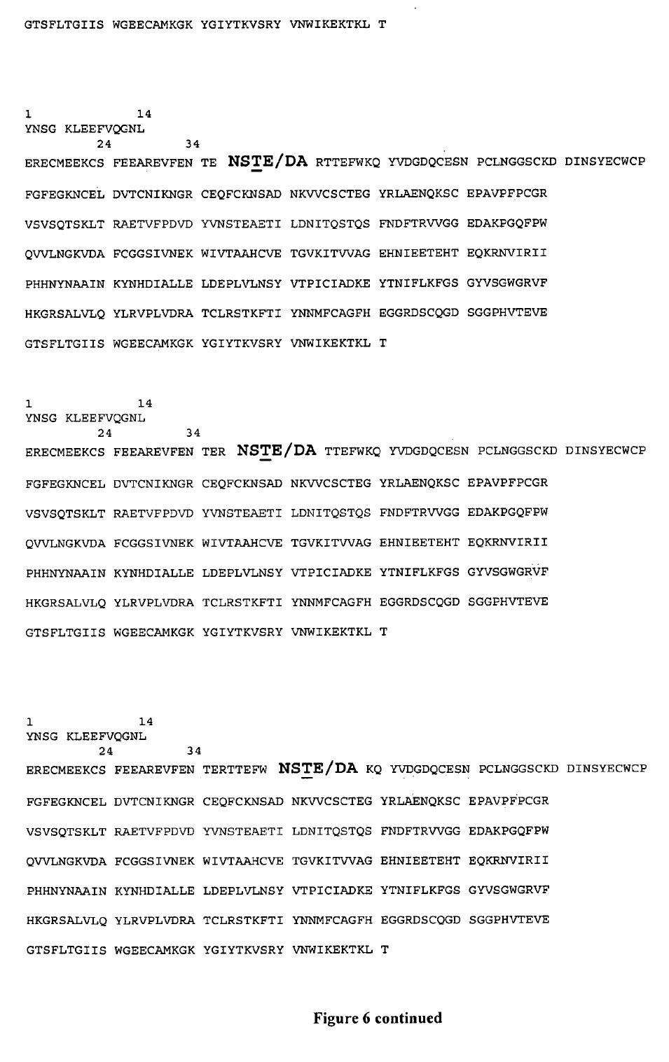

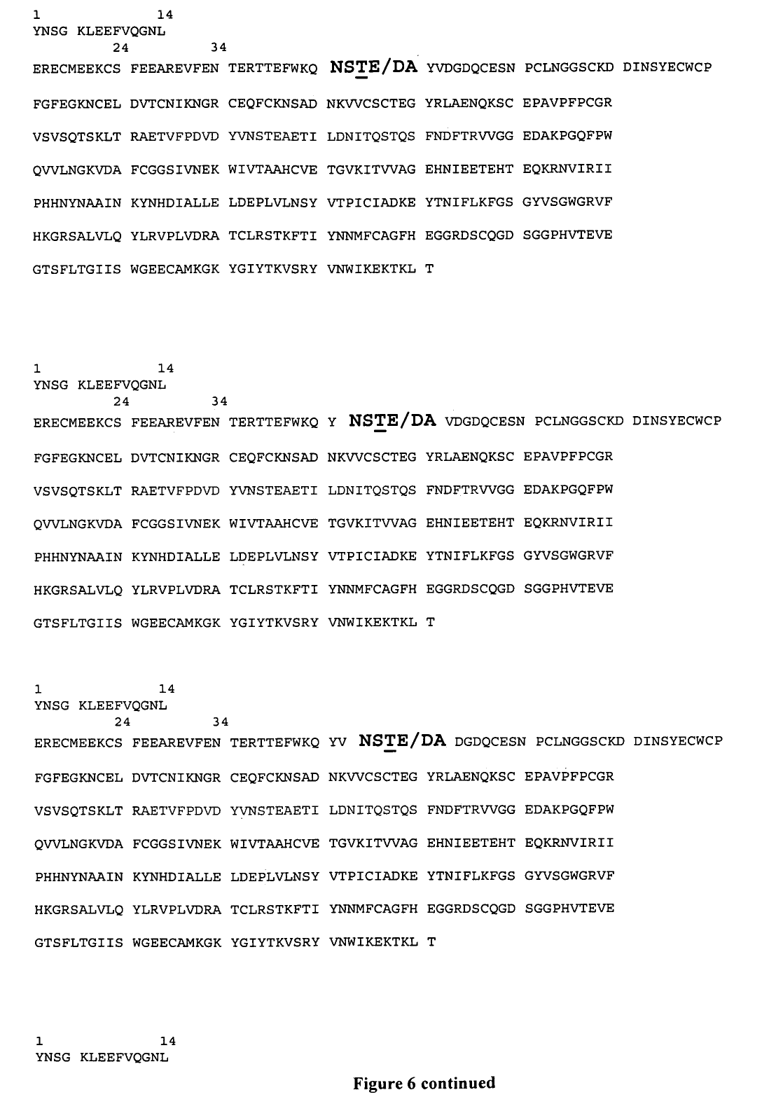

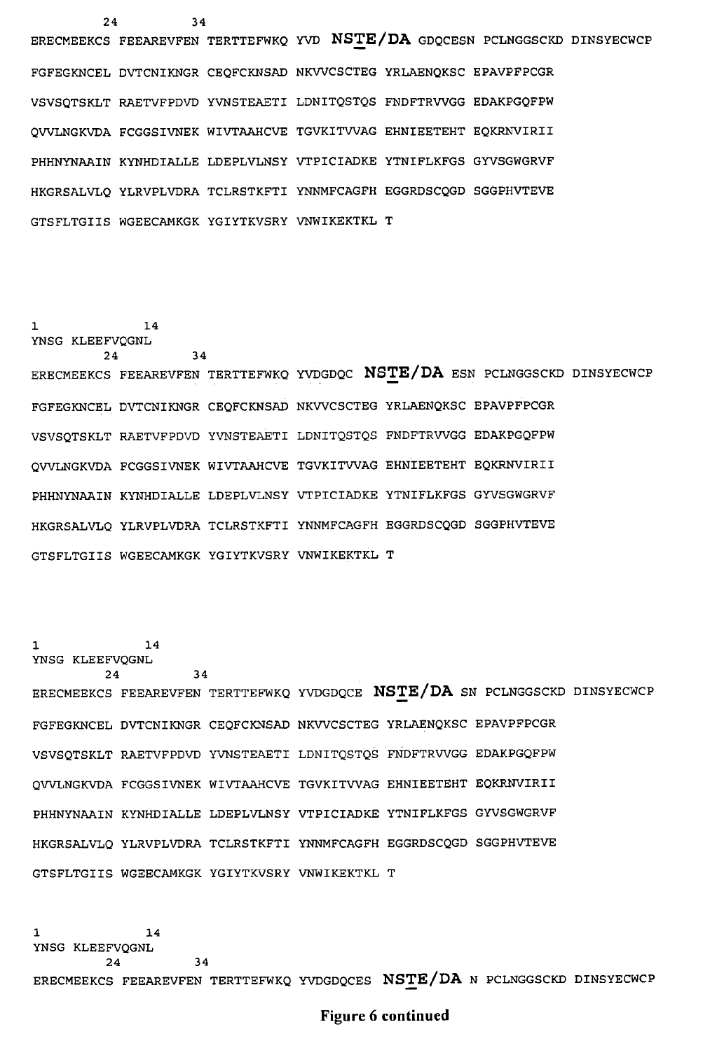

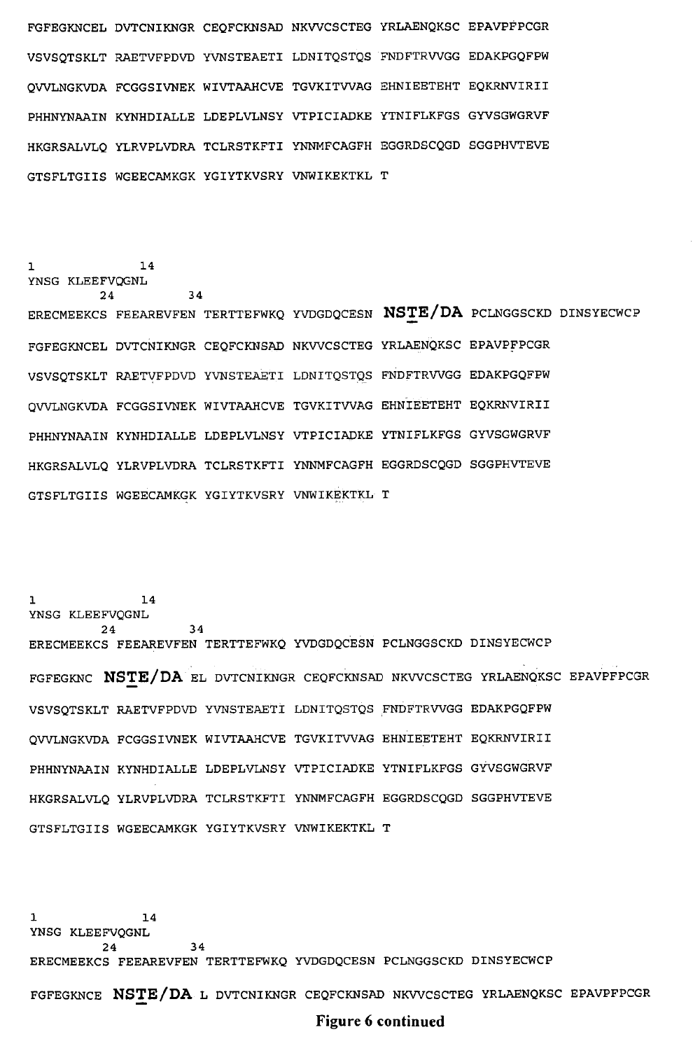

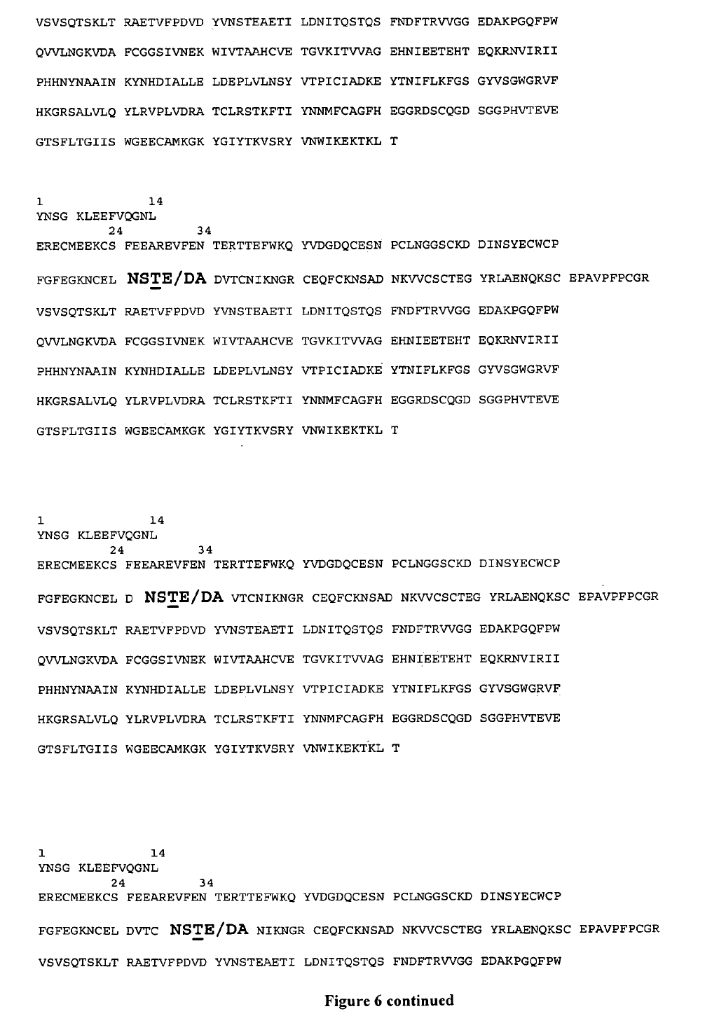

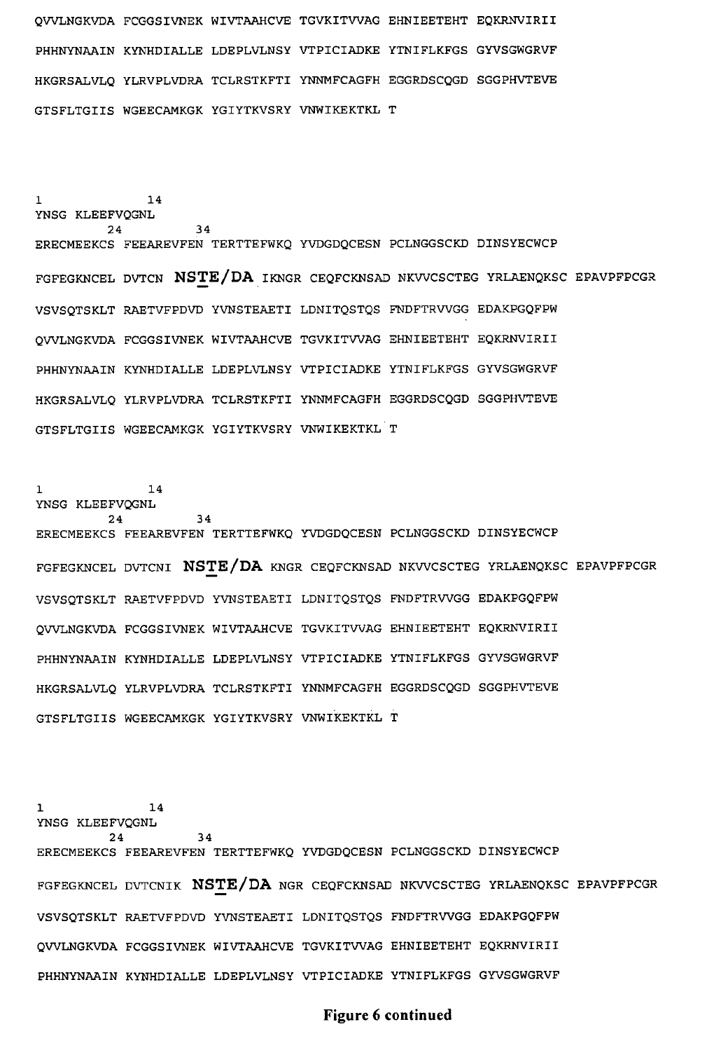

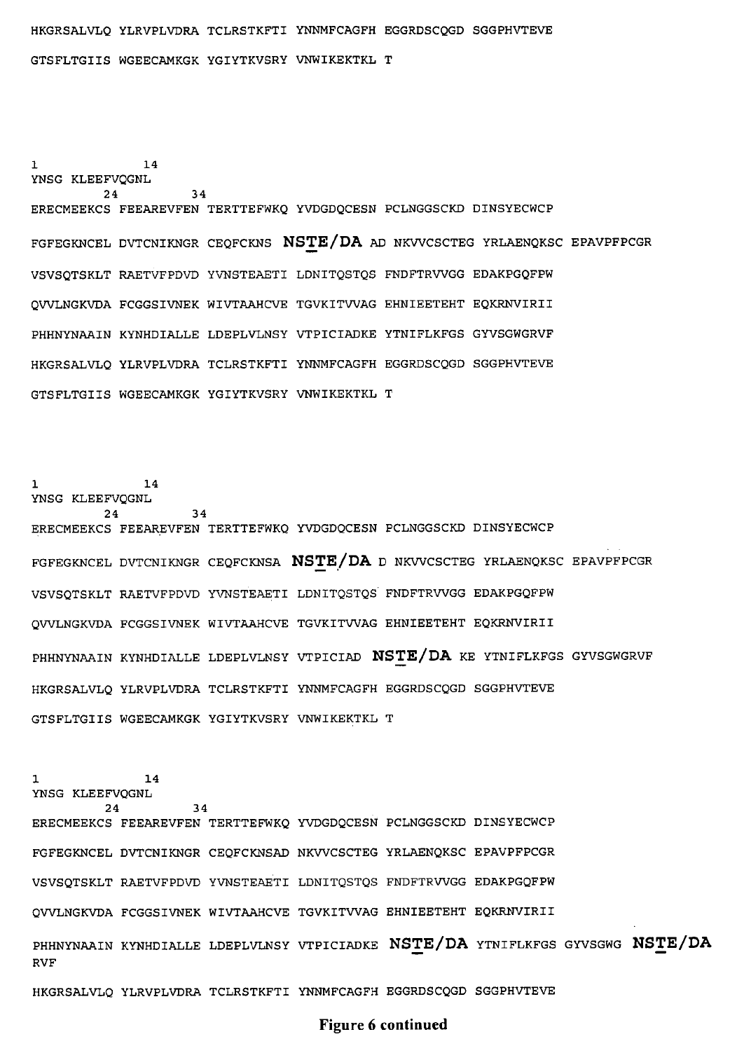

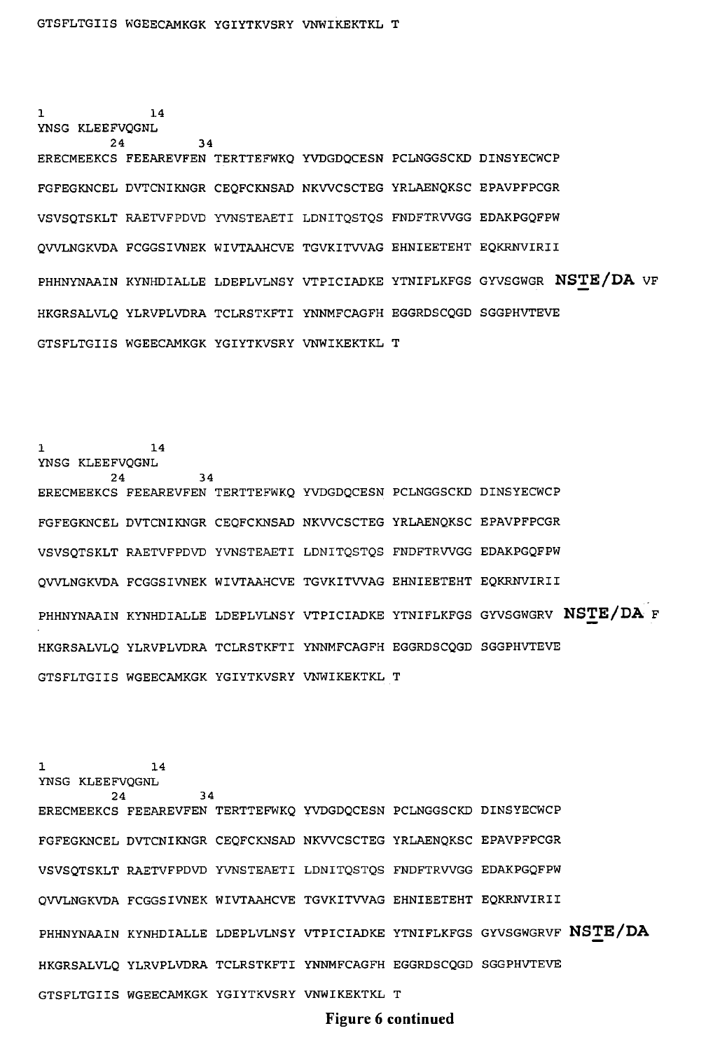

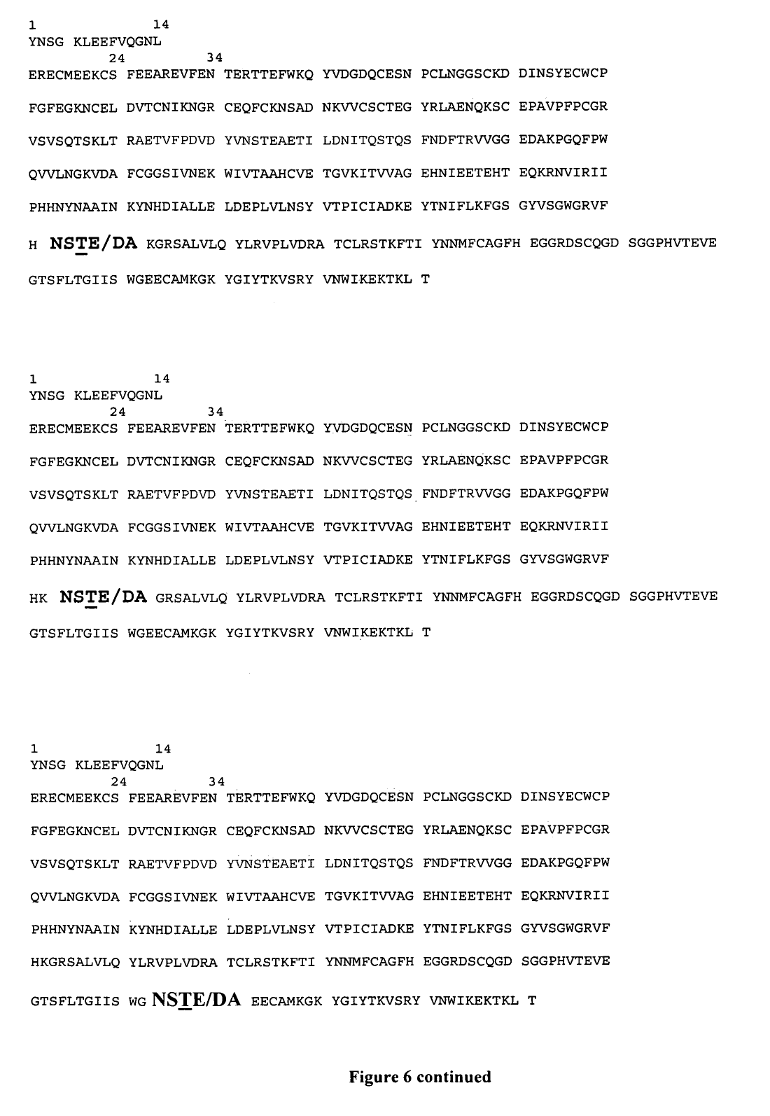

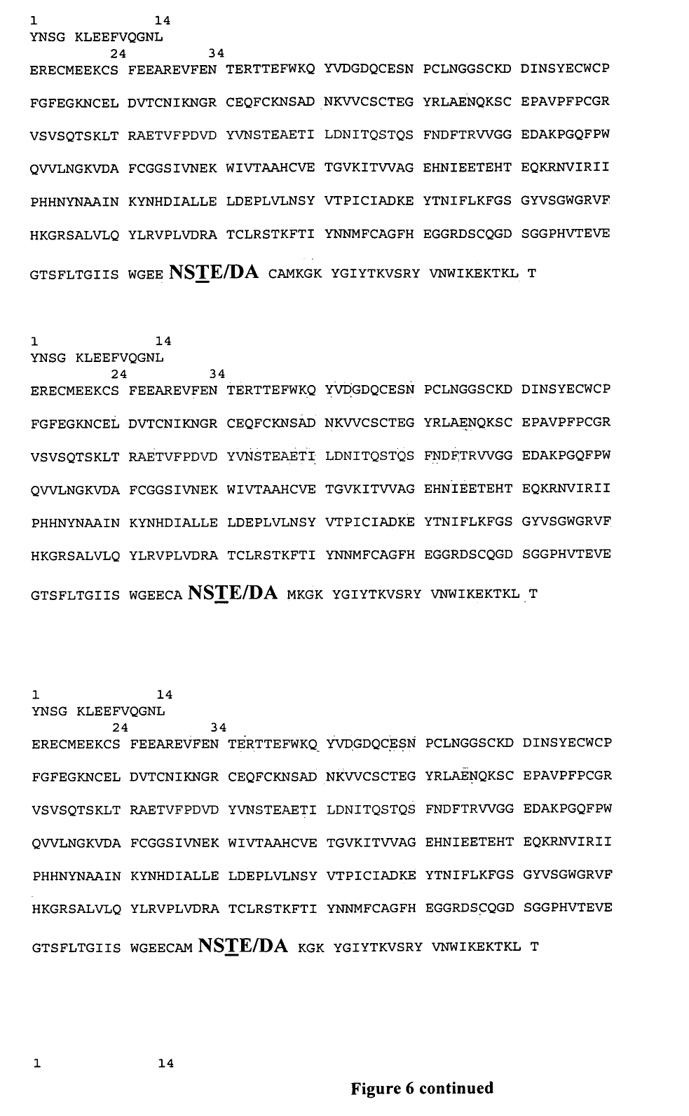

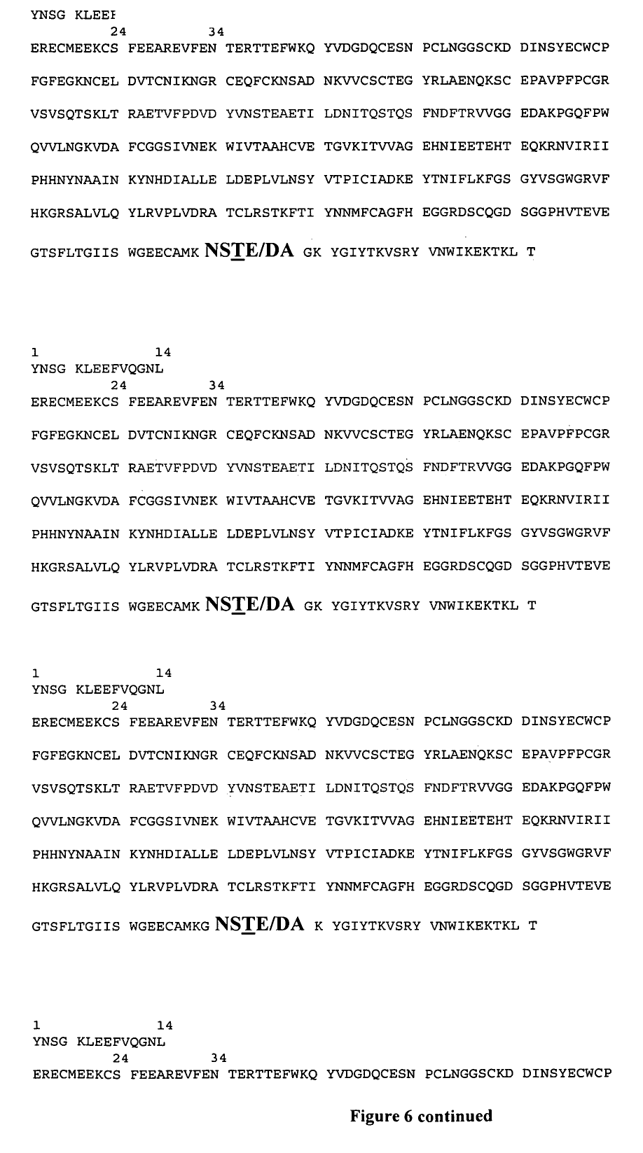

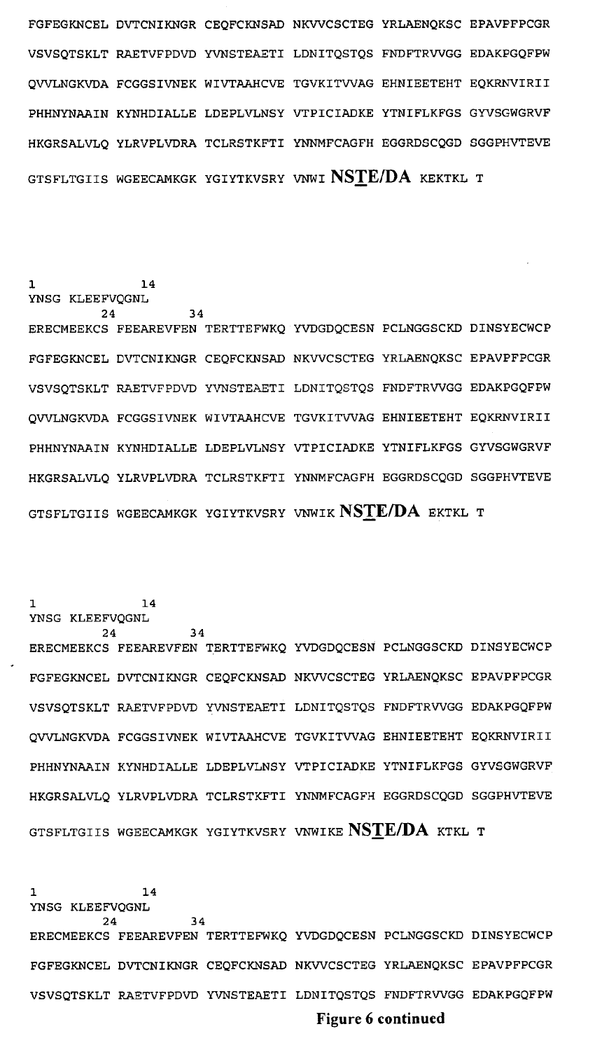

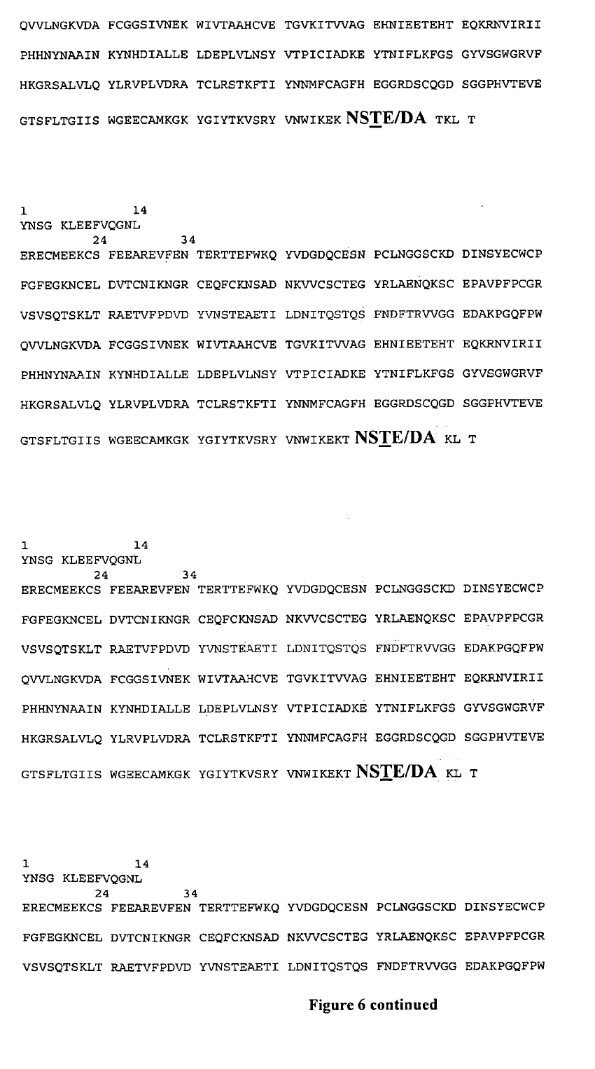

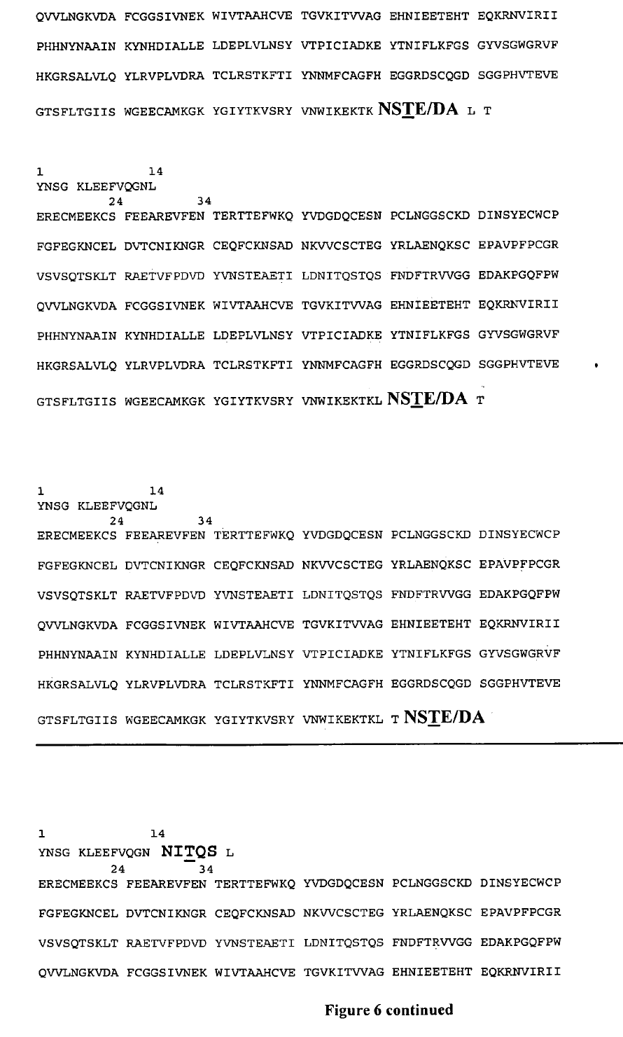

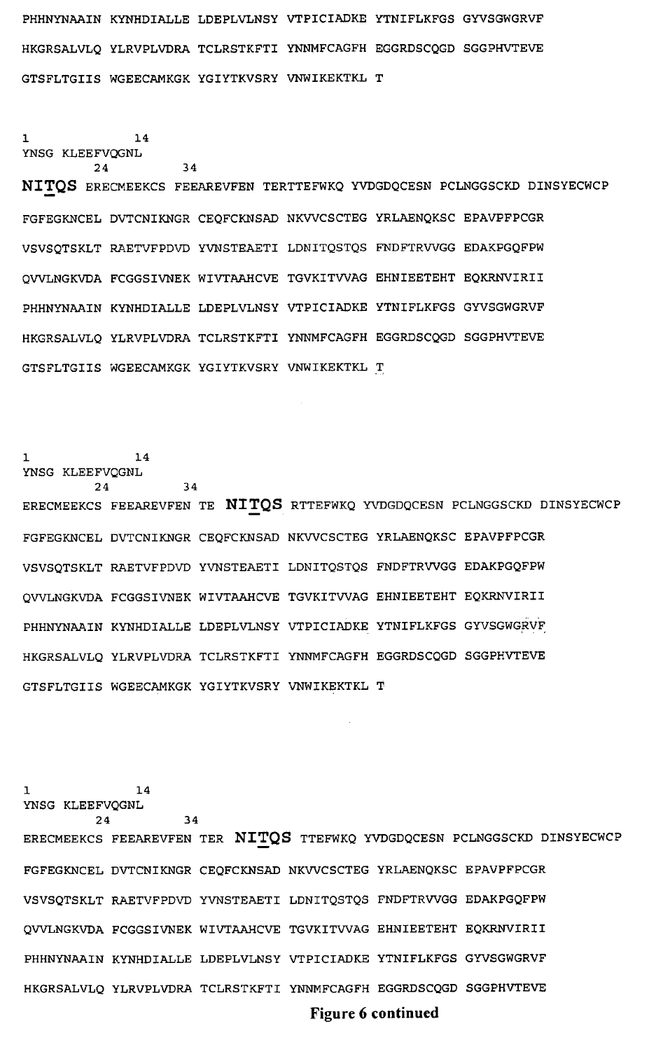

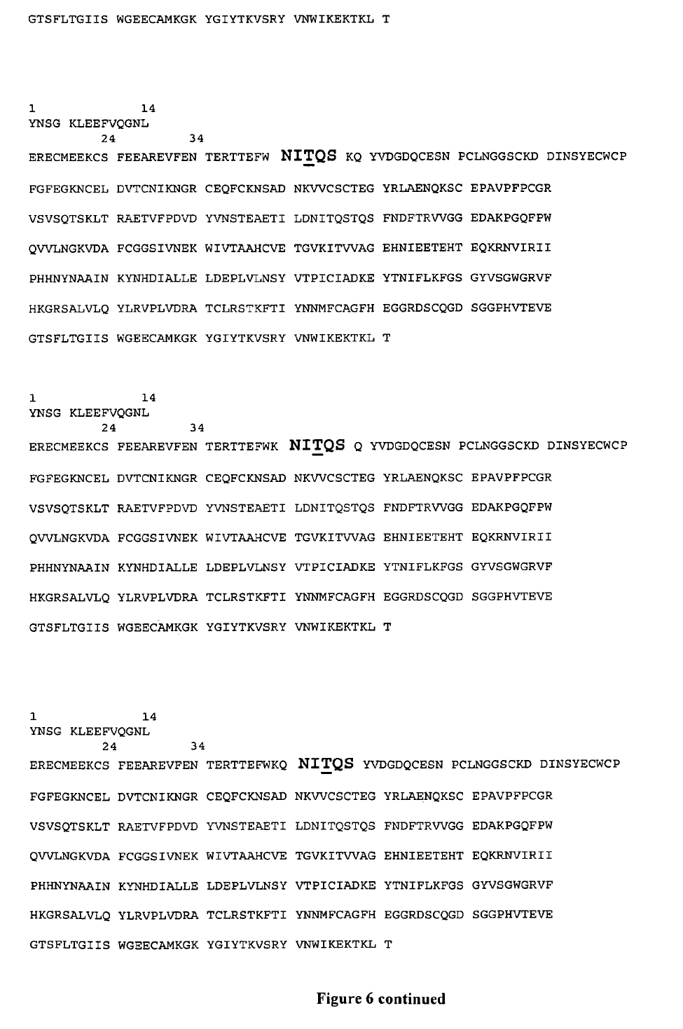

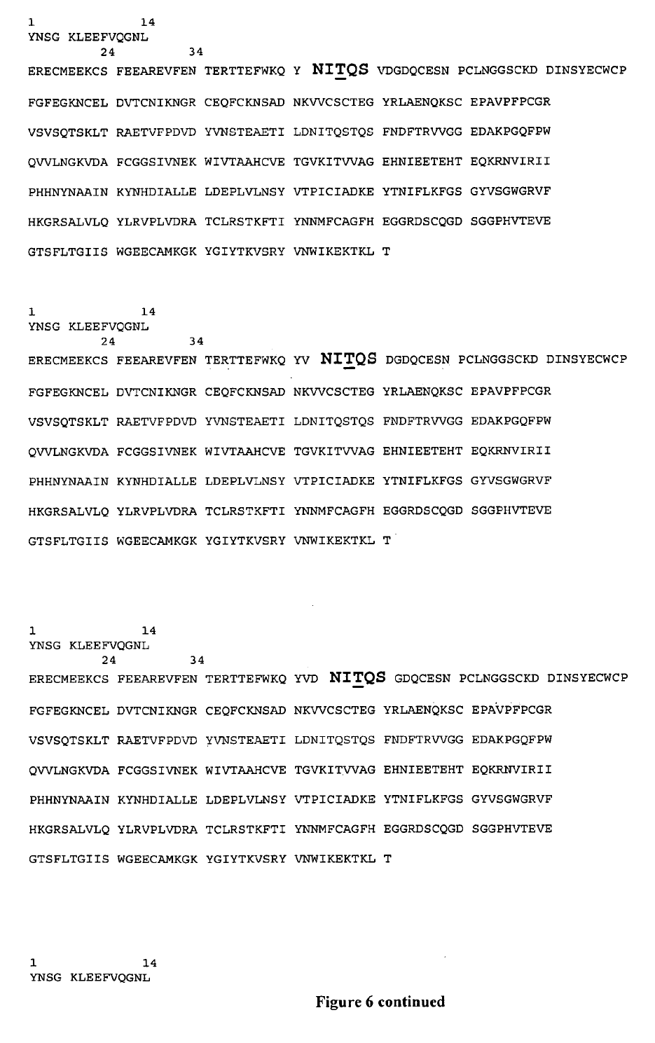

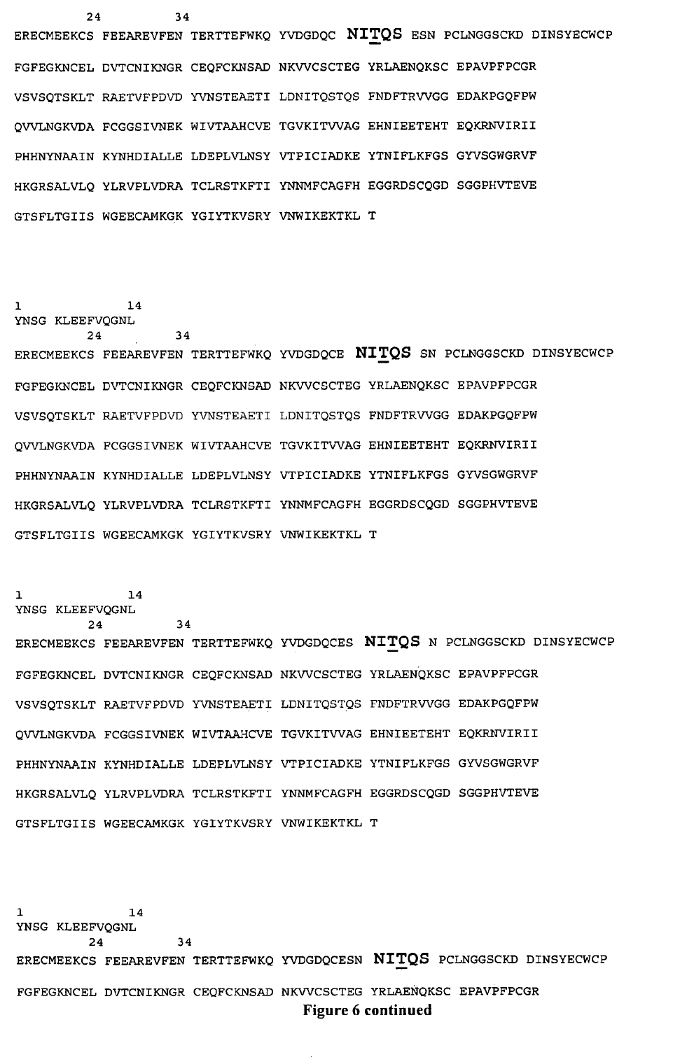

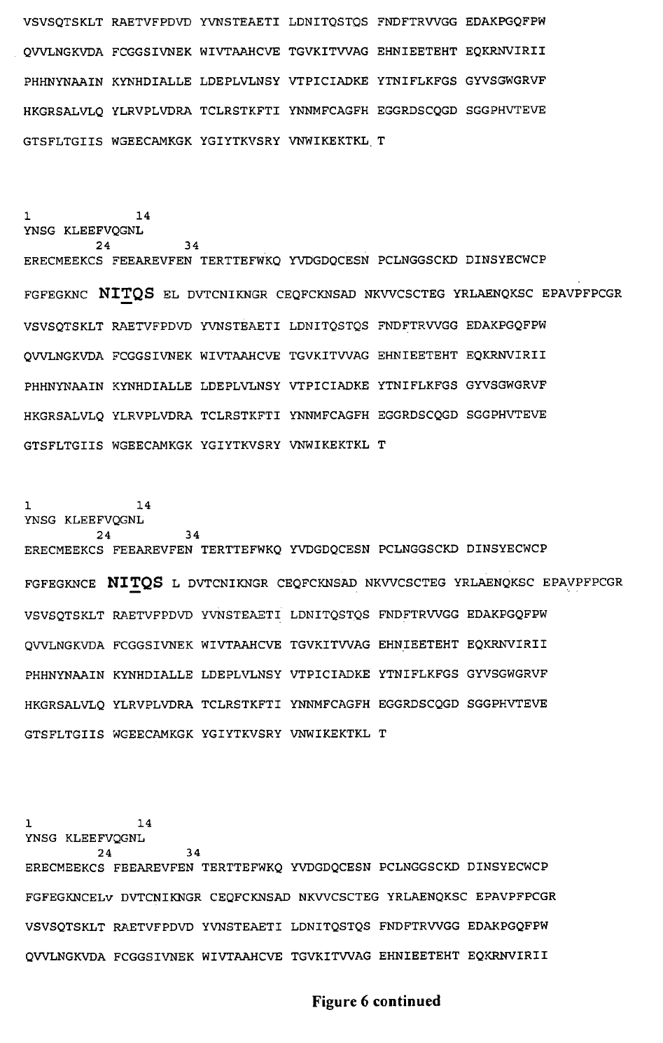

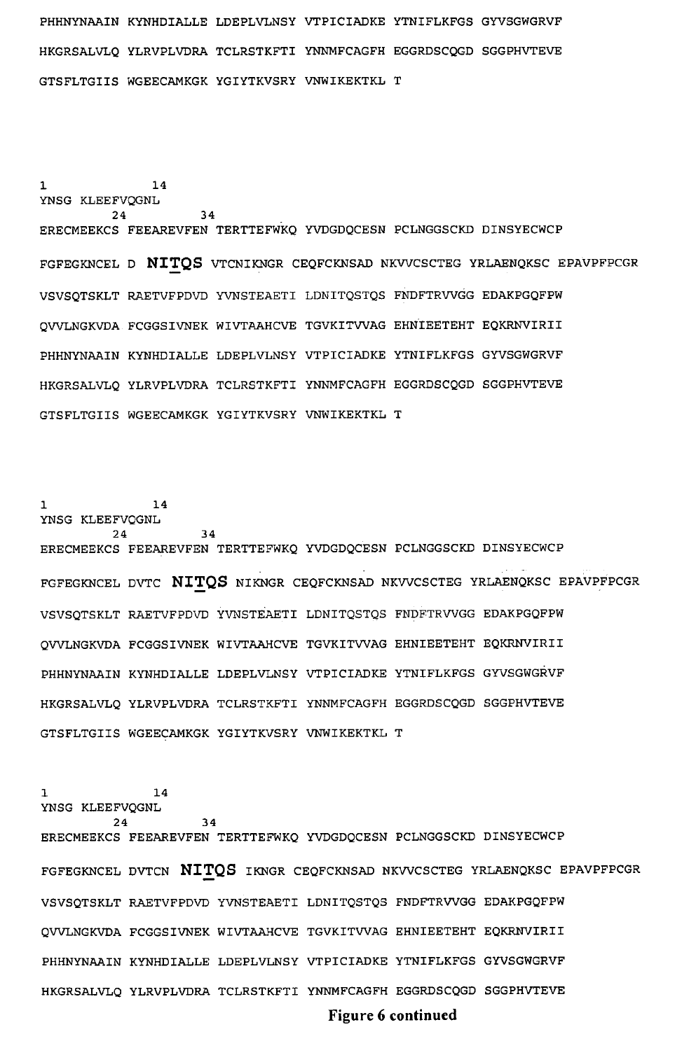

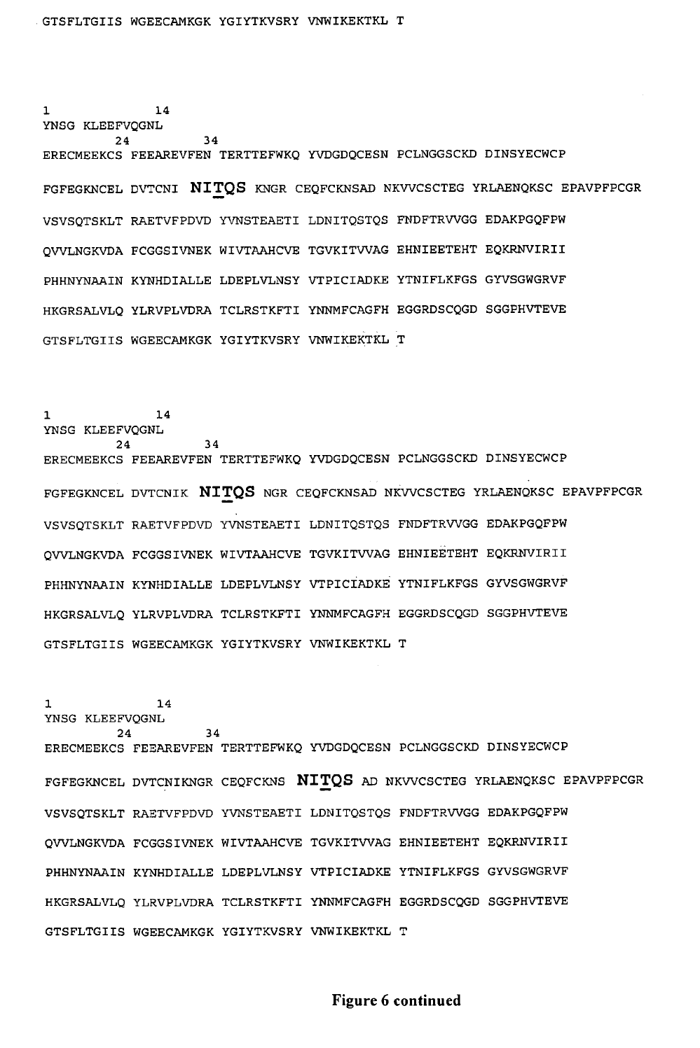

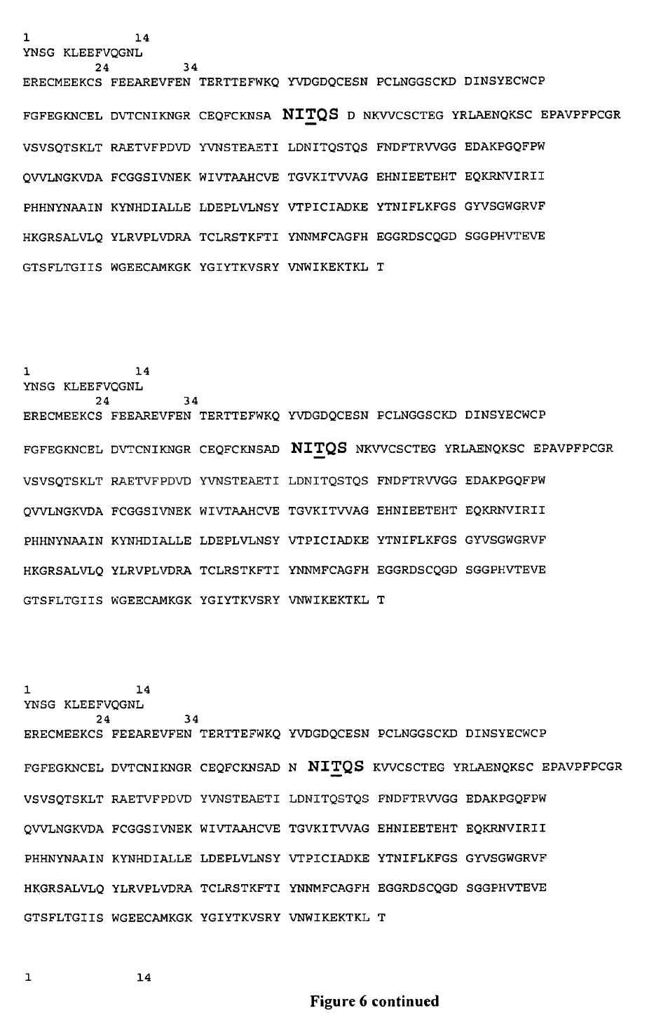

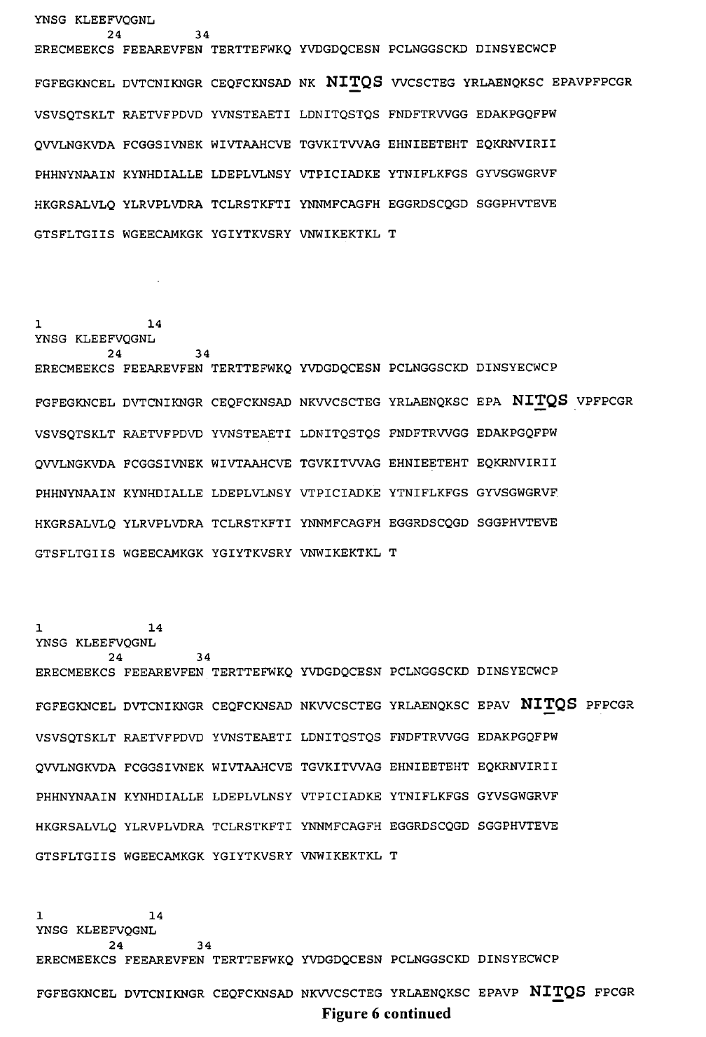

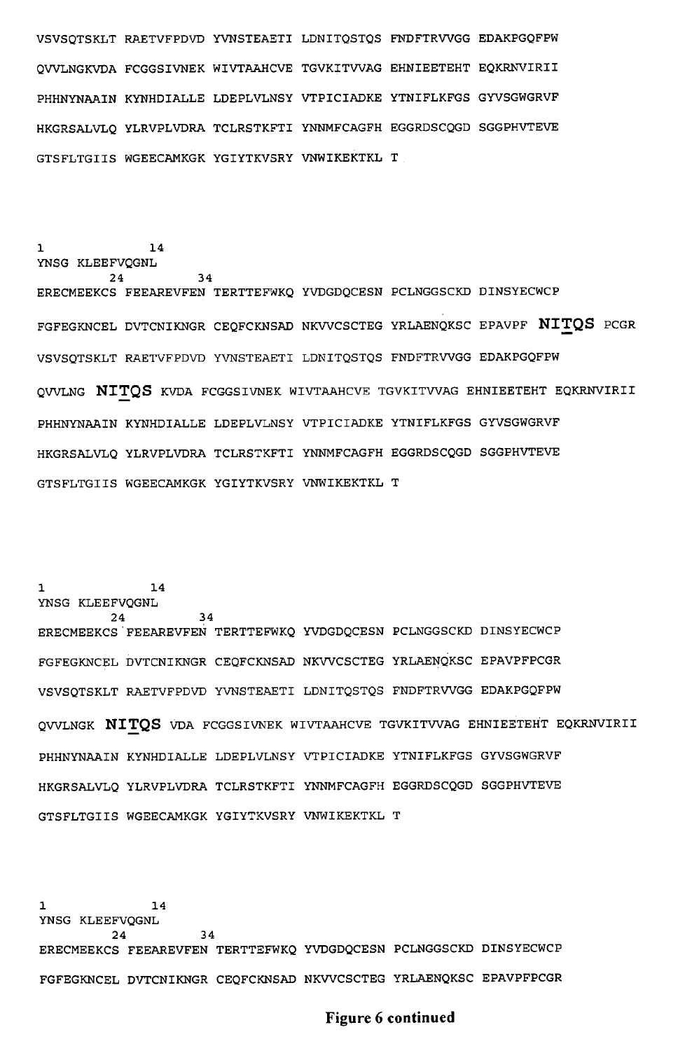

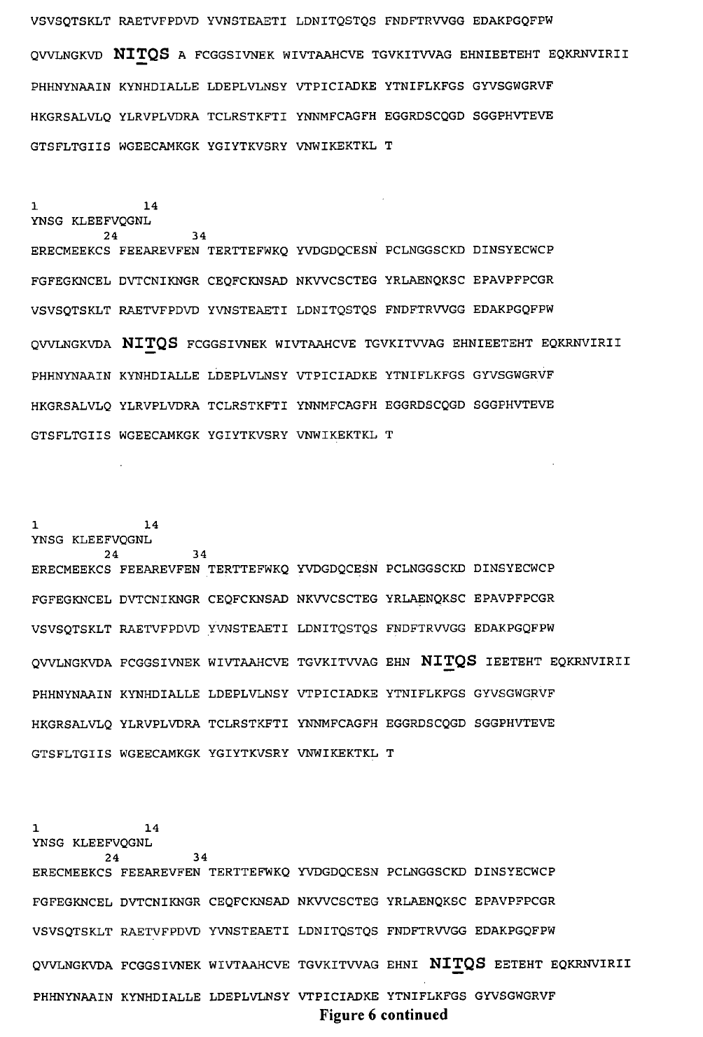

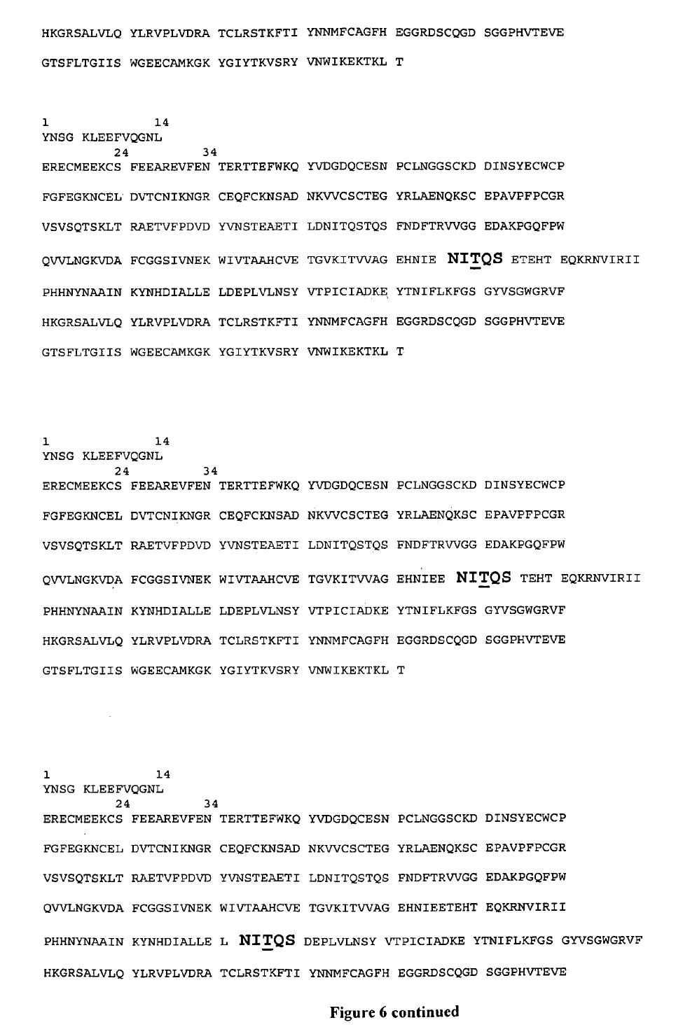

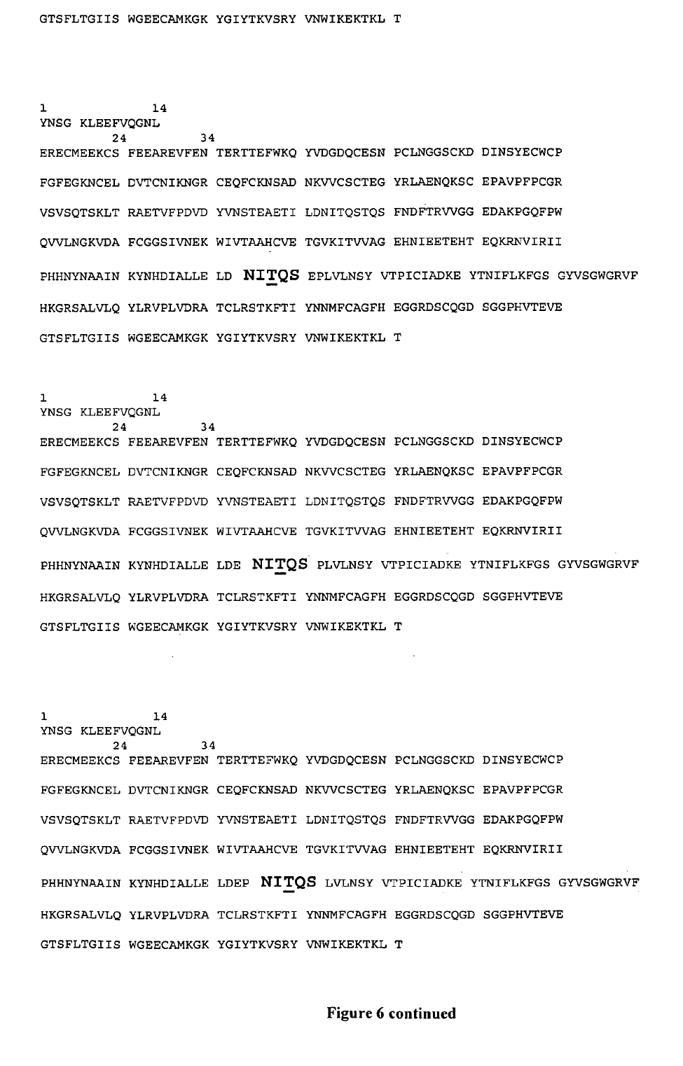

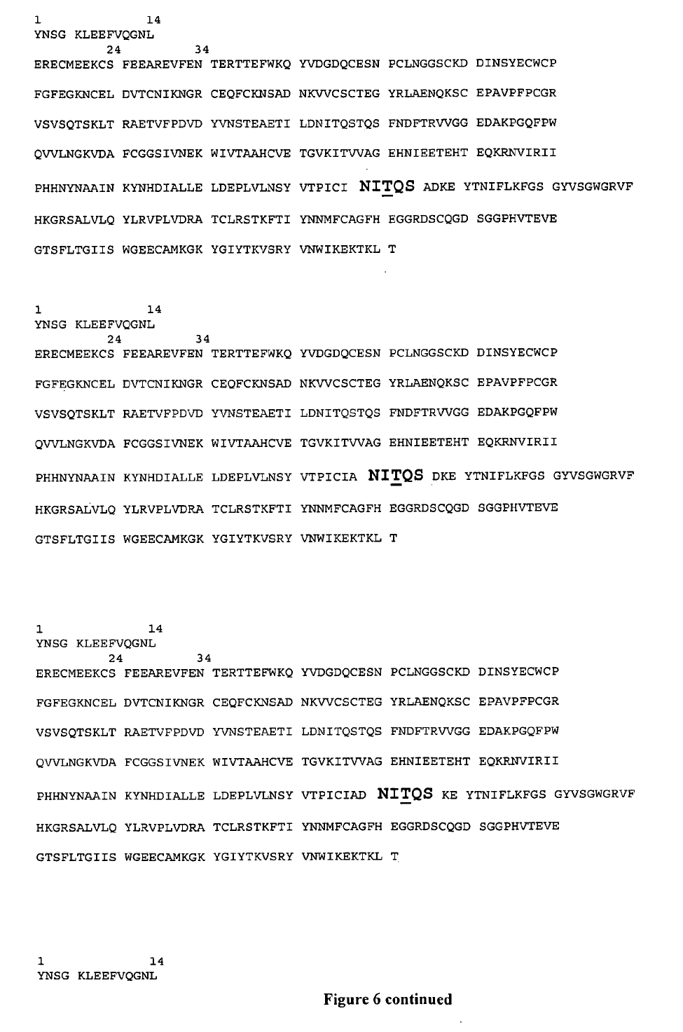

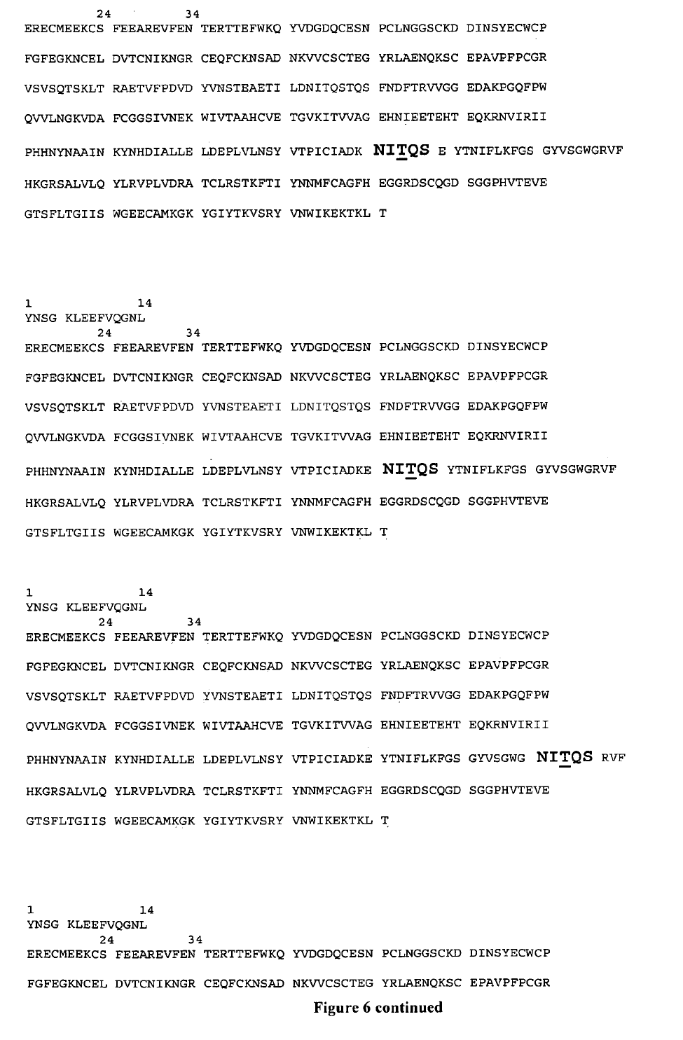

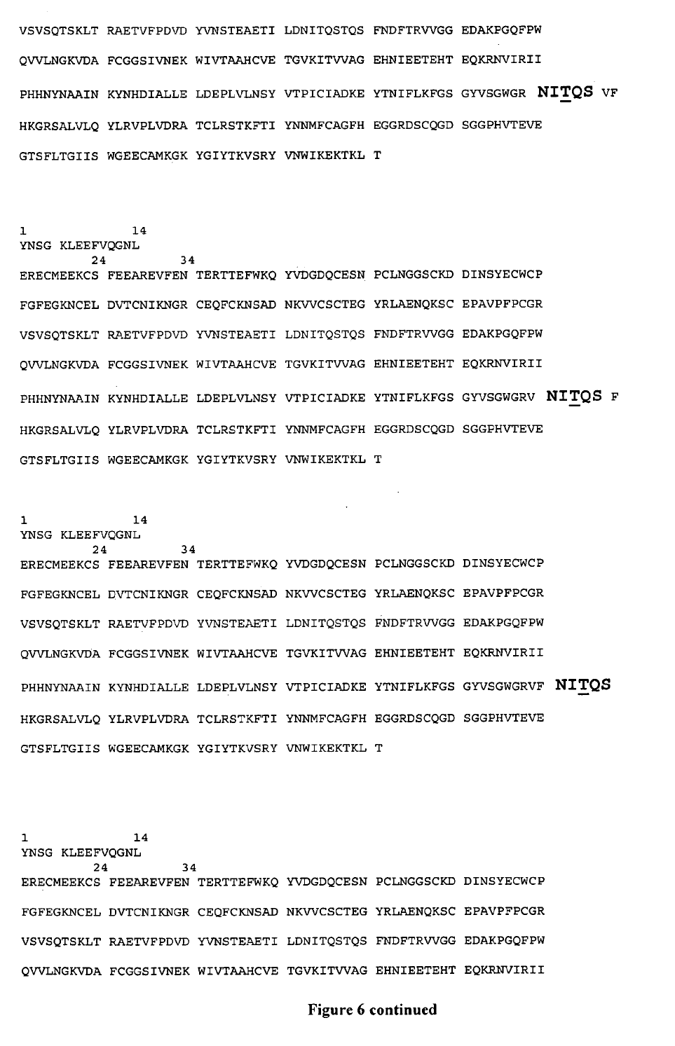

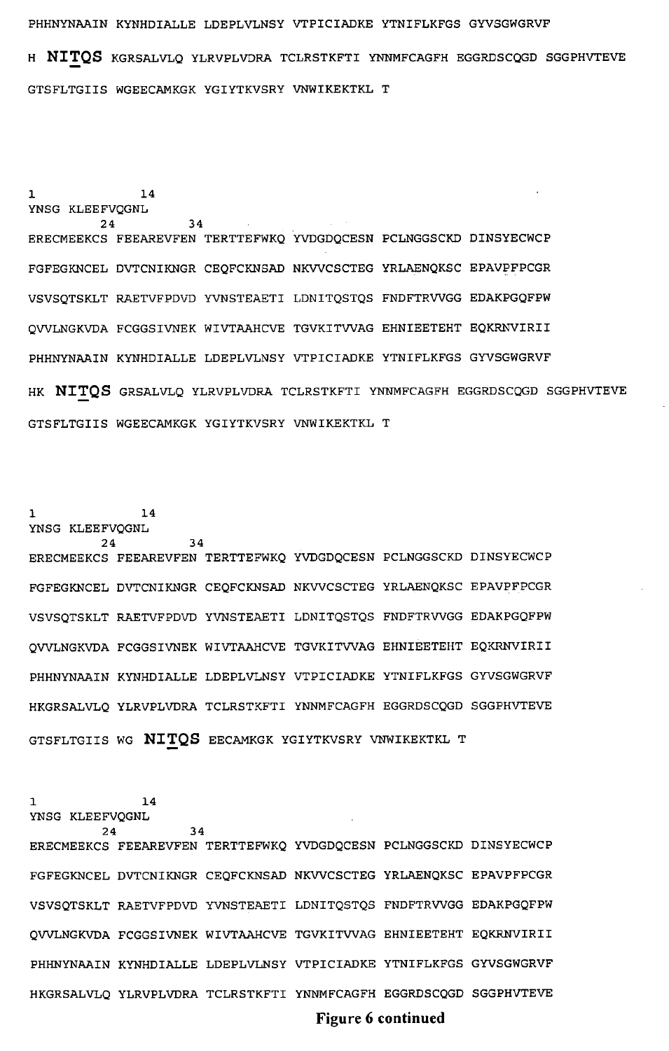

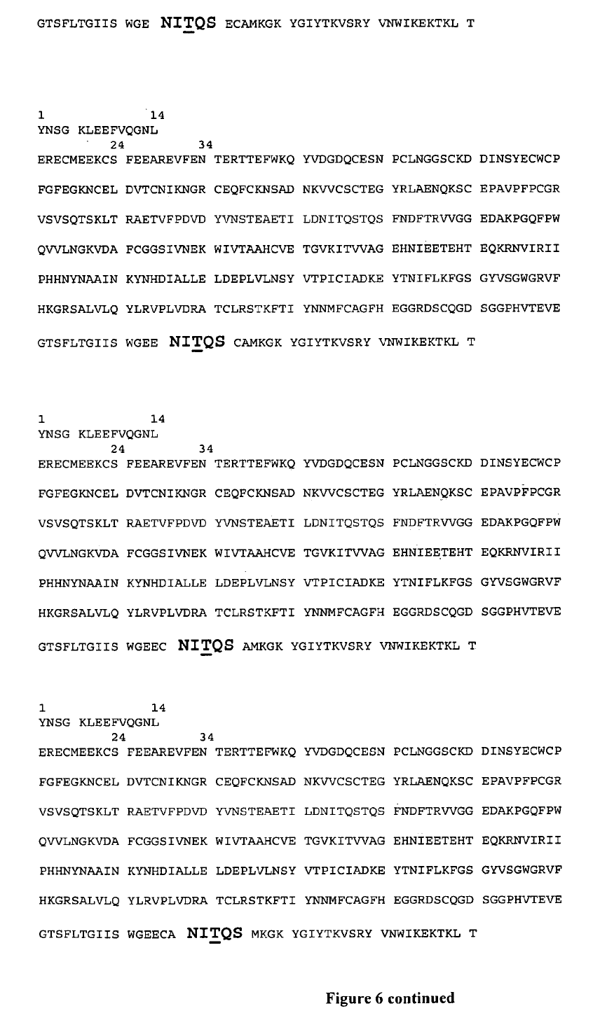

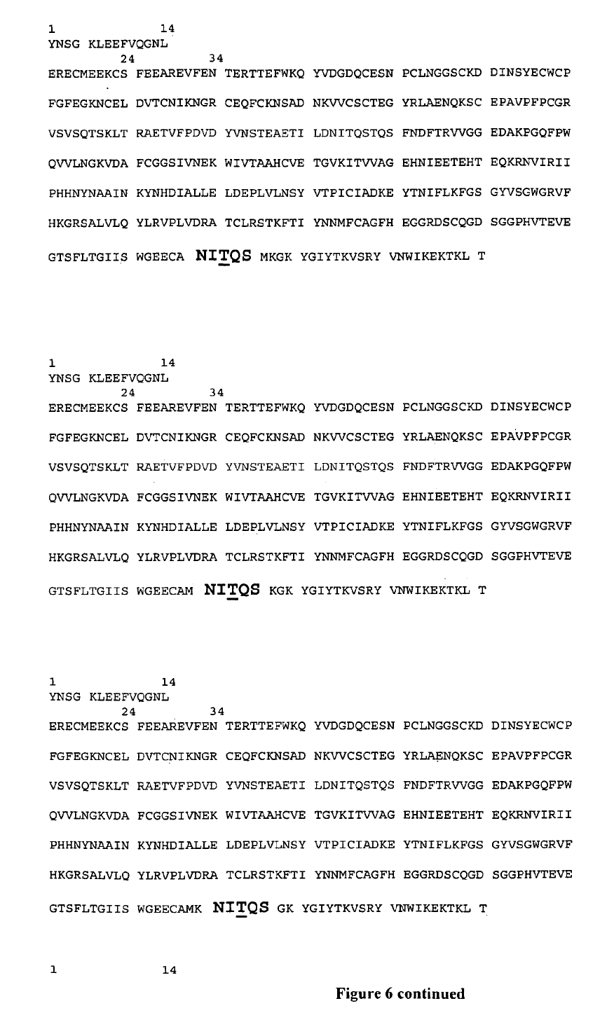

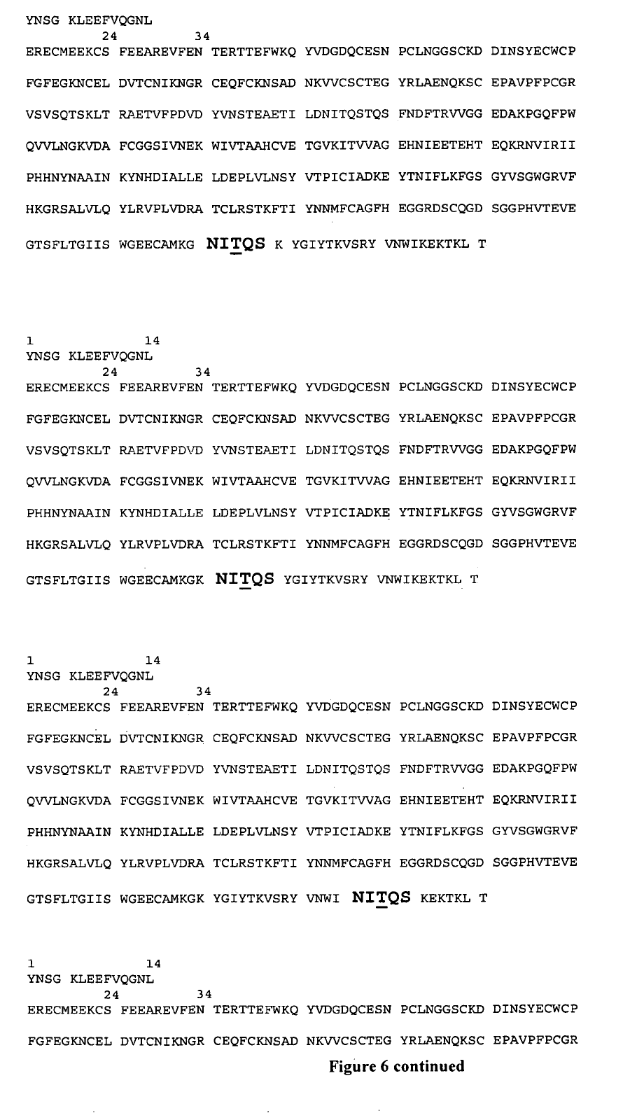

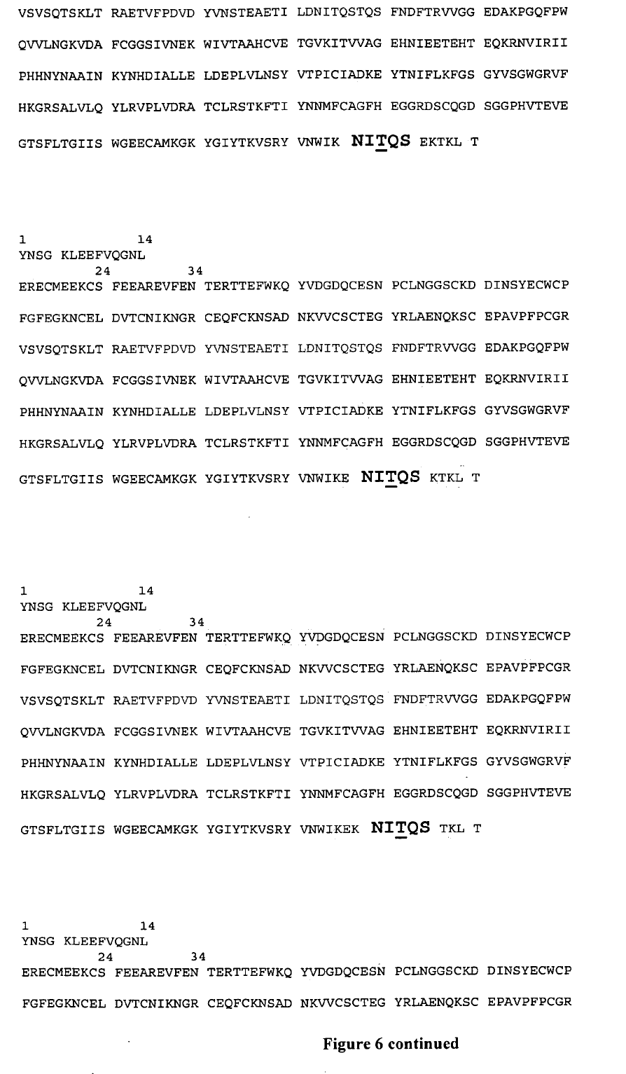

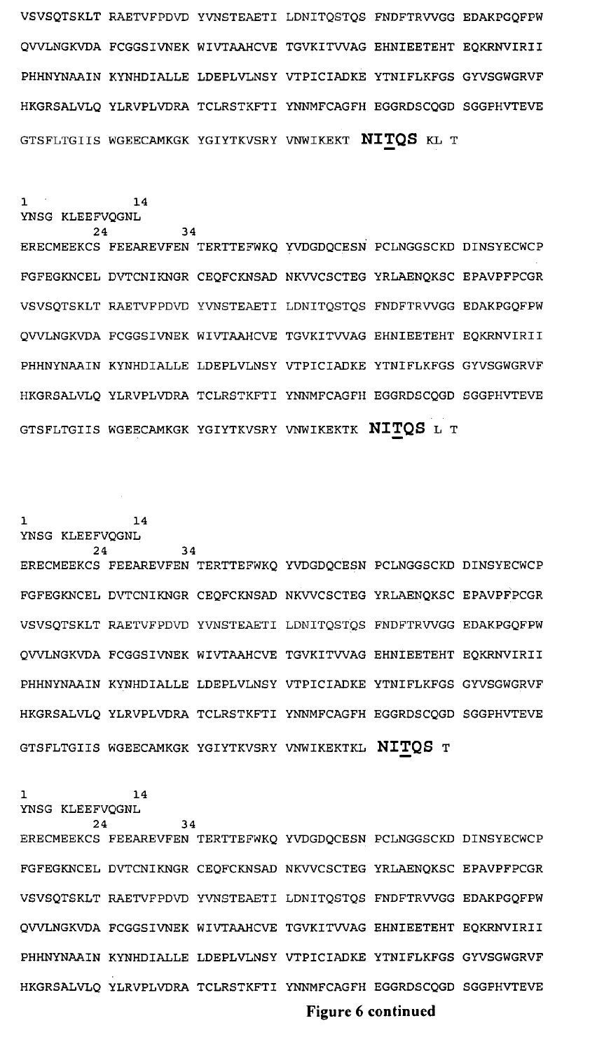

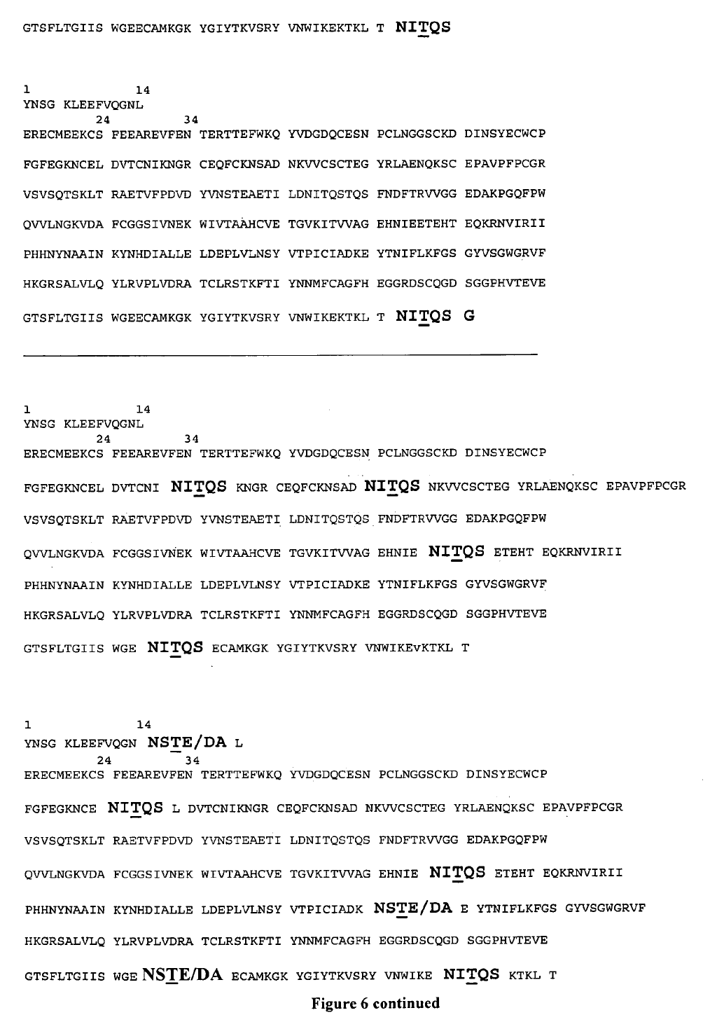

Numerous examples of human FIX variants with one or more additional N-linked glycosylation site outside the activation peptide or human FIX variants with combinations of additional N-linked and O-linked glycosylation sites are provided herein as SEQ ID NOs:135-304. Numerous other examples of human FIX variants with one or more additional O-linked glycosylation sites outside the activation peptide are provided herein in FIG. 6. It would be readily understood that the modifications shown in FIG. 6 and in the Sequence Listing can be combined with modifications shown in any of the other examples provided herein and that the modifications shown in FIG. 6 and in the Sequence Listing are not limited to the specific N-linked or O-linked glycosylation site consensus sequences shown. Any of the N-linked and/or O-linked glycosylation site consensus sequences of this invention as well as any others known in the art are included within the embodiments of this invention and can be introduced into the FIX of this invention individually, in any combination with other O-linked glycosylation site consensus sequences and/or with any N-linked glycosylation site consensus sequences to increase the number of glycosylation sites on the FIX protein.

Additional embodiments of the invention are direct to methods of increasing the number of glycosylation sites in a Factor IX protein, comprising one or more of the following steps: a) aligning a first and a second Factor IX amino acid sequence; b) identifying one or more glycosylation sites in the first FIX amino acid sequence that are not present in the second FIX amino acid sequence; and c) altering the second FIX amino acid sequence to introduce one or more new or additional glycosylation sites in the second FIX amino acid sequence corresponding to the one or more glycosylation sites identified in the first amino acid sequence in step (b). In particular embodiments, the first amino acid sequence is Factor IX from a non-human species and the second amino acid sequence is human Factor IX. In certain embodiments, the one or more new or additional glycosylation sites are introduced into the activation peptide of the second FIX amino acid sequence. In other embodiments, the one or more new or additional glycosylation sites are introduced outside the activation peptide of the second FIX amino acid sequence and in further embodiments, the one or more new or additional glycosylation sites are introduced both in the activation peptide of the second FIX amino acid sequence and outside the activation peptide of the second FIX amino acid sequence, in any combination and at any location. In the methods of this invention, the new or additional glycosylation sites can be N-linked and/or O-linked glycosylation sites in any combination.

The methods of this invention comprise modifying the second FIX amino acid sequence within the vicinity of a corresponding region containing a glycosylation site in the first FIX amino acid sequence (e.g., within 1, 2, 3, 4, 5 or 6 amino acids), as well as modifying the second FIX amino acid sequence at the exact amino acid position(s) as those in the corresponding region in the first FIX amino acid sequence.

Additionally provided herein is a nucleic acid comprising, consisting essentially of and/or consisting of a nucleotide sequence encoding a FIX amino acid sequence of this invention. Such nucleic acids can be present in a vector, such as an expression cassette. Thus, further embodiments of the invention are directed to expression cassettes designed to express a nucleotide sequence encoding any of the Factor IX variants of this invention. The nucleic acids and/or vectors of this invention can be present in a cell. Thus, various embodiments of the invention are directed to recombinant host cells containing the vector (e.g., expression cassette). Such a cell can be isolated and/or present in a transgenic animal. Therefore, certain embodiments of the invention are further directed to a transgenic animal comprising a nucleic acid comprising a nucleotide sequence encoding any of the Factor IX variants of the present invention.

A comparison of the amino acid sequence of the activation peptide of human, mouse, guinea pig and platypus FIX reveals that the mouse FIX amino acid sequence has an additional nine amino acids present in its activation peptide, the guinea pig FIX amino acid sequence has an additional ten amino acid residues present in its activation peptide and the platypus has an additional 14 amino acid residues present in its activation peptide (FIG. 5). These extra amino acids are between the two naturally occurring glycosylation sites (N 157 and N 167) in human Factor IX.

The human and mouse FIX have essentially identical structures and the human enzyme can function in the mouse. As the human FIX functions without the additional nine amino acid segment found in the mouse, this region of the Factor IX molecule can tolerate modifications within its sequence, including insertions, substitutions and/or deletions, without substantial loss in structural, biochemical, or otherwise functional integrity of the molecule. The inserted nine amino acids in mouse are most likely surface residues (as supported by structural studies) and therefore accessible for modification by the glycosylation enzymes. In native human factor IX, the two N-linked glycosylation sites are 12 and 14 amino acids distant from the amino and carboxyl cleavage sites, respectively, of the activation peptide. Thus, in some embodiments of the invention, additional amino acid residues can be added between N157 and N167 of the human Factor IX protein in order to add glycosylation sites to improve half life and/or bioavailability. In various embodiments, glycosylation sites are added by insertion, deletion and/or modification of the native sequence to include an attachment sequence for O-linked glycosylation and/or consensus sequences for N-linked glycosylation.

The human sequence for the activation peptide starts at residue 146 of the mature protein. The natural glycosylation sites are at N157 and N167 (SEQ ID NO:33). In some embodiments, additional amino acid residues can be inserted between the two normal glycosylation sites (between N157 and N167 in the human sequence) to provide additional glycosylation sites. In some embodiments, about 3 to about 100 additional amino acid residues are added. In other embodiments, about 5 to about 50 amino acid residues are added. In further embodiments, about 5 to about 20 amino acid residues are added. In yet further embodiments, about 7 to about 15 amino acid residues are added. Typically, the amino acid residues are chosen from the 20 biological amino acids with the proviso that proline is not used as “X” in the glycosylation site NXT/S, which is the consensus sequence for N-linked glycosylation. Table 1 shows 20 common biological amino acids and their abbreviations.

N-glycosylation sites and/or O-glycosylation sites may be added. Consensus sequences for addition of glycosylation sites are known in the art and include the consensus sequence “NXT/S” for N-glycosylation where X is not proline. O-glycosylation sites are more varied and generally do not have a “consensus sequence” for attachment. In preferred embodiments, additional O-linked glycosylation sites for Factor IX are introduced by insertion, deletion and/or modification of the native sequence to include consensus sequences for O-linked glycosylation found in other clotting proteins such as Factor II, Factor VII, Factor VIII, Factor X, Protein C, and Protein S. For example, the sequence CXXGGT/S-C (SEQ ID NO:9) is found in several clotting factors and hemostatic proteins as a consensus sequence for attaching an O-linked oligosaccharide (van den Steen et al. In Critical Reviews in Biochemistry and Molecular Biology, Michael Cox, ed., 33(3):151-208 (1998)). In some embodiments, the glycosylation site(s) include O-linked glycosylation site(s) including but not limited to:

| | |
|---|---|
| CXXGGT/S-C | (SEQ ID NO: 9) |
| NSTE/DA | (SEQ ID NO: 10) |
| NITQS | (SEQ ID NO: 11) |
| QSTQS | (SEQ ID NO: 12) |
| D/E-FT-R/K-V | (SEQ ID NO: 13) |
| C-E/D-SN | (SEQ ID NO: 14) |
| GGSC-K/R | (SEQ ID NO: 15) |

In the sequences above, the attachment point for glycosylation is underlined. In some embodiments, the FIX variant is prepared by insertion of the S/T residue for O-linked glycosylation with a residue on either side such as the following trimers: G-T/S-C, ST-E/D, ITQ, STQ, FT-R/K, E/D-SN and GSC. Other variations include the interchangeability of S and T for the actual glycosylation site. S may be substituted for T and T may be substituted for S. Embodiments of the invention are directed to the addition, by insertion, deletion and/or substitution, of any sequence thought to be a signal for either N-linked or O-linked glycosylation.

In some embodiments, endogenous N-linked and O-linked attachment sequences from mouse, human and other mammalian Factor IX sequences are inserted into the activation peptide. These may be inserted individually or in combination. In certain embodiments, the inserted segment includes a spacer region between glycosylation sites, which can be present individually, in tandem repeats, in multiples, etc. A spacer region of this invention can be from one to about 100 amino acids in length (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and 100). In some embodiments, for example, the spacer region can be from one to about 20 amino acids. In other embodiments the spacer region can be from one to about ten amino acids. In further embodiments, the spacer region can be from one to about five amino acid residues.

A spacer region of this invention is included between the added carbohydrate attachment sites and/or between naturally occurring glycosylation sites and added glycosylation sites to reduce or eliminate steric hindrance and provide efficient recognition by the appropriate glycosyltransferase. A spacer region of this invention can be comprised of any combination of amino acid residues provided that they are not cysteine or proline and provided that the amino acid sequence of the spacer does not have more than about 10% residues that are hydrophobic (e.g., tryptophan, tyrosine, phenylalanine and valine).

In some embodiments, NXT/S is incorporated into the inserted amino acid sequence to add one or more additional glycosylation sites. “X” may be any biological amino acid except that proline is disfavored. In some embodiments, at least one additional glycosylation site is added to the Factor IX variant. In other embodiments, two additional glycosylation sites are added. In further embodiments, three additional glycosylation sites are added. In yet further embodiments, four additional glycosylation sites are added. In further embodiments, five additional glycosylation sites are added. In some embodiments, six additional glycosylation sites are added. In other embodiments, more than six additional glycosylation sites are added.

In one embodiment, Ala at position 161 of the mature human FIX amino acid sequence (SEQ ID NO:33) is replaced with Asn to provide one additional glycosylation site. In another embodiment, a peptide segment from the mouse activation peptide is inserted into the human FIX activation peptide with modification of the mouse sequence to create one additional glycosylation site (FIG. 1, line 3; SEQ ID NO:3) or two additional glycosylation sites (SEQ ID NO:4; FIG. 1, line 2). In a further embodiment, the sequence VFIQDNITD (SEQ ID NO:6) is inserted between residues 161 and 162 of the mature human FIX amino acid sequence of SEQ ID NO:33 to introduce an N-linked glycosylation site in the human FIX sequence. In yet a further embodiment, another new glycosylation site is added by replacing Asp with Asn in the VFIQDNITD insert. The inserted sequence would give VFIQDNITN (SEQ ID NO:7). The embodiments discussed above could be combined to provide one to four additional glycosylation sites in the human Factor IX protein.

In another embodiment, the following sequence is added, which provides five additional glycosylation sites. The glycosylation sites are shown in bold and underlined.

(SEQ ID NO: 8)
AETVFPDVDYVNSTENETIQDNITDNETILDNITQSTQSFNDFTR

In some embodiments, glycosylation sites are added at sites outside of the activation peptide. These additional sites can be selected, for example, by aligning the amino acid sequence of Factor IX from human with the Factor IX amino acid sequence from other species and determining the position of glycosylation sites in non-human species. The homologous or equivalent position in the human FIX amino acid sequence is then modified to provide a glycosylation site. This method may be used to identify both potential N-glycosylation and O-glycosylation sites.

An example of this approach is provided in FIG. 2, in which the human FIX amino acid sequence (SEQ ID NO:1) is aligned with the FIX amino acid sequence from dog, pig, cow and mouse, respectively. At the position of the fifth star, there is a glycosylation site in all species shown except human, indicating that glycosylation is well tolerated at this position. The dog, pig, cow and mouse FIX amino acid sequences have the consensus sequence for N-glycosylation at this site (NXT/S), but the human FIX amino acid sequence does not. Instead the human FIX amino acid sequence is NAA. Mutation of the human FIX amino acid sequence at amino acid 262 to produce the consensus sequence NAT/S will introduce an additional glycosylation site at this position in the human FIX protein.

The FIX variants according to the invention are produced and characterized by methods well known in the art and as described in the EXAMPLE section provided herein. These methods include determination of clotting time (partial thromboplastin time (PPT) assay) and administration of the FIX variant to a test animal to determine recovery, half life, and bioavailability by an appropriate immunoassay and/or activity-assay, as are well known in the art.

In some embodiments, a recombinant Factor IX protein is produced by one or more of the method steps described herein. The recombinant Factor IX protein produced by the methods described can be included in a pharmaceutical composition. Some embodiments are directed to a kit which includes the recombinant Factor IX protein produced according to the methods described herein. The recombinant Factor IX protein can be used in a method of treating bleeding disorders by administering an effective amount of the recombinant Factor IX protein to a subject (e.g., a human patient) in need thereof.

Many expression vectors can be used to create genetically engineered cells. Some expression vectors are designed to express large quantities of recombinant proteins after amplification of transfected cells under a variety of conditions that favor selected, high expressing cells. Some expression vectors are designed to express large quantities of recombinant proteins without the need for amplification under selection pressure. The present invention includes the production of genetically engineered cells according to methods standard in the art and is not dependent on the use of any specific expression vector or expression system.

To create a genetically engineered cell to produce large quantities of a Factor IX protein, cells are transfected with an expression vector that contains the cDNA encoding the protein. In some embodiments, the target protein is expressed with selected co-transfected enzymes that cause proper post-translational modification of the target protein to occur in a given cell system.

The cell may be selected from a variety of sources, but is otherwise a cell that may be transfected with an expression vector containing a nucleic acid, preferably a cDNA encoding a Factor IX protein.

The practice of the present invention employs, unless otherwise indicated, conventional techniques of molecular biology, microbiology, recombinant DNA, and immunology, which are within the skill of the art. Such techniques are explained fully in the literature. See, e.g., Sambrook, et al., Molecular Cloning; A Laboratory Manual, 2nd ed. (1989); DNA Cloning, Vols. I and II (D. N Glover, ed. 1985); Oligonucleotide Synthesis (M. J. Gait, ed. 1984); Nucleic Acid Hybridization (B. D. Hames & S. J. Higgins, eds. 1984); Transcription and Translation (B. D. Hames & S. J. Higgins, eds. 1984); Animal Cell Culture (R. I. Freshney, ed. 1986); Immobilized Cells and Enzymes (IRL Press, 1986); B. Perbal, A Practical Guide to Molecular Cloning (1984); the series, Methods in Enzymology (Academic Press, Inc.), particularly Vols. 154 and 155 (Wu and Grossman, and Wu, eds., respectively); Gene Transfer Vectors for Mammalian Cells (J. H. Miller and M. P. Calos, eds. 1987, Cold Spring Harbor Laboratory); Immunochemical Methods in Cell and Molecular Biology, Mayer and Walker, eds. (Academic Press, London, 1987); Scopes, Protein Purification: Principles and Practice, 2nd ed. 1987 (Springer-Verlag, N.Y.); and Handbook of Experimental Immunology Vols I-IV (D. M. Weir and C. C. Blackwell, eds 1986). All patents, patent applications, and publications cited in the specification are incorporated herein by reference in their entireties.

### Genetic Engineering Techniques

The production of cloned genes, recombinant DNA, vectors, transformed host cells, proteins and protein fragments by genetic engineering is well known. See, e.g., U.S. Pat. No. 4,761,371 to Bell et al. at Col. 6, line 3 to Col. 9, line 65; U.S. Pat. No. 4,877,729 to Clark et al. at Col. 4, line 38 to Col. 7, line 6; U.S. Pat. No. 4,912,038 to Schilling at Col. 3, line 26 to Col. 14, line 12; and U.S. Pat. No. 4,879,224 to Wallner at Col. 6, line 8 to Col. 8, line 59.

A vector is a replicable DNA construct. Vectors are used herein either to amplify nucleic acid encoding Factor IX Protein and/or to express nucleic acid which encodes Factor IX Protein. An expression vector is a replicable nucleic acid construct in which a nucleotide sequence encoding a Factor IX protein is operably linked to suitable control sequences capable of effecting the expression the nucleotide sequence to produce a Factor IX protein in a suitable host. The need for such control sequences will vary depending upon the host selected and the transformation method chosen. Generally, control sequences include a transcriptional promoter, an optional operator sequence to control transcription, a sequence encoding suitable mRNA ribosomal binding sites, and sequences that control the termination of transcription and translation.

Vectors comprise plasmids, viruses (e.g., adenovirus, cytomegalovirus), phage, and integratable DNA fragments (i.e., fragments integratable into the host genome by recombination). The vector replicates and functions independently of the host genome, or may, in some instances, integrate into the genome itself. Expression vectors can contain a promoter and RNA binding sites that are operably linked to the gene to be expressed and are operable in the host organism.

DNA regions or nucleotide sequences are operably linked or operably associated when they are functionally related to each other. For example, a promoter is operably linked to a coding sequence if it controls the transcription of the sequence; or a ribosome binding site is operably linked to a coding sequence if it is positioned so as to permit translation of the sequence.

Transformed host cells are cells which have been transformed, transduced and/or transfected with Factor IX protein vector(s) constructed using recombinant DNA techniques.

Suitable host cells include prokaryote, yeast or higher eukaryotic cells such as mammalian cells and insect cells. Cells derived from multicellular organisms are a particularly suitable host for recombinant Factor IX protein synthesis, and mammalian cells are particularly preferred. Propagation of such cells in cell culture has become a routine procedure (Tissue Culture, Academic Press, Kruse and Patterson, editors (1973)). Examples of useful host cell lines are VERO and HeLa cells, Chinese hamster ovary (CHO) cell lines, and WI138, HEK 293, BHK, COS-7, CV, and MDCK cell lines. Expression vectors for such cells ordinarily include (if necessary) an origin of replication, a promoter located upstream from the nucleotide sequence encoding Factor IX protein to be expressed and operatively associated therewith, along with a ribosome binding site, an RNA splice site (if intron-containing genomic DNA is used), a polyadenylation site, and a transcriptional termination sequence. In a preferred embodiment, expression is carried out in Chinese Hamster Ovary (CHO) cells using the expression system of U.S. Pat. No. 5,888,809, which is incorporated herein by reference in its entirety.

The transcriptional and translational control sequences in expression vectors to be used in transforming vertebrate cells are often provided by viral sources. For example, commonly used promoters are derived from polyoma, Adenovirus 2, and Simian Virus 40 (SV40). See. e.g., U.S. Pat. No. 4,599,308.

An origin of replication may be provided either by construction of the vector to include an exogenous origin, such as may be derived from SV 40 or other viral (e.g., polyoma, adenovirus, VSV, or BPV) source, or may be provided by the host cell chromosomal replication mechanism. If the vector is integrated into the host cell chromosome, the latter is often sufficient.

Rather than using vectors which contain viral origins of replication, one can transform mammalian cells by the method of cotransformation with a selectable marker and the nucleic acid encoding the Factor IX protein. Examples of suitable selectable markers are dihydrofolate reductase (DHFR) or thymidine kinase. This method is further described in U.S. Pat. No. 4,399,216 which is incorporated by reference herein in its entirety.

Other methods suitable for adaptation to the synthesis of Factor IX protein in recombinant vertebrate cell culture include those described in Gething et al. Nature 293:620 (1981); Mantei et al. Nature 281:40; and Levinson et al., EPO Application Nos. 117,060A and 117,058A, the entire contents of each of which are incorporated herein by reference.

Host cells such as insect cells (e.g., cultured Spodoptera frugiperda cells) and expression vectors such as the baculovirus expression vector (e.g., vectors derived from Autographa californica MNPV, Trichoplusia ni MNPV, Rachiplusia ou MNPV, or Galleria ou MNPV) may be employed in carrying out the present invention, as described in U.S. Pat. Nos. 4,745,051 and 4,879,236 to Smith et al. In general, a baculovirus expression vector comprises a baculovirus genome containing the nucleotide sequence to be expressed inserted into the polyhedrin gene at a position ranging from the polyhedrin transcriptional start signal to the ATG start site and under the transcriptional control of a baculovirus polyhedrin promoter.

Prokaryote host cells include gram negative or gram positive organisms, for example Escherichia coli (E. coli) or bacilli. Higher eukaryotic cells include established cell lines of mammalian origin as described below. Exemplary host cells are E. coli W3110 (ATCC 27,325), E. coli B, E. coli X1776 (ATCC 31,537) and E. coli 294 (ATCC 31,446). A broad variety of suitable prokaryotic and microbial vectors are available. E. coli is typically transformed using pBR322. Promoters most commonly used in recombinant microbial expression vectors include the betalactamase (penicillinase) and lactose promoter systems (Chang et al. Nature 275:615 (1978); and Goeddel et al. Nature 281:544 (1979)), a tryptophan (trp) promoter system (Goeddel et al. Nucleic Acids Res. 8:4057 (1980) and EPO App. Publ. No. 36,776) and the tac promoter (De Boer et al. Proc. Natl. Acad. Sci. USA 80:21 (1983)). The promoter and Shine-Dalgarno sequence (for prokaryotic host expression) are operably linked to the nucleic acid encoding the Factor IX protein, i.e., they are positioned so as to promote transcription of Factor IX messenger RNA from DNA.

Eukaryotic microbes such as yeast cultures may also be transformed with protein-encoding vectors (see, e.g., U.S. Pat. No. 4,745,057). Saccharomyces cerevisiae is the most commonly used among lower eukaryotic host microorganisms, although a number of other strains are commonly available. Yeast vectors may contain an origin of replication from the 2 micron yeast plasmid or an autonomously replicating sequence (ARS), a promoter, nucleic acid encoding Factor IX protein, sequences for polyadenylation and transcription termination, and a selection gene. An exemplary plasmid is YRp7, (Stinchcomb et al. Nature 282:39 (1979); Kingsman et al. Gene 7:141 (1979); Tschemper et al. Gene 10:157 (1980)). Suitable promoting sequences in yeast vectors include the promoters for metallothionein, 3-phosphoglycerate kinase (Hitzeman et al. J. Biol. Chem. 255:2073 (1980) or other glycolytic enzymes (Hess et al. J. Adv. Enzyme Reg. 7:149 (1968); and Holland et al. Biochemistry 17:4900 (1978)). Suitable vectors and promoters for use in yeast expression are further described in R. Hitzeman et al., EPO Publn. No. 73,657.

Cloned coding sequences of the present invention may encode FIX of any species of origin, including mouse, rat, dog, opossum, rabbit, cat, pig, horse, sheep, cow, guinea pig, opossum, platypus, and human, but preferably encode Factor IX protein of human origin. Nucleic acid encoding Factor IX that is hybridizable with nucleic acid encoding proteins disclosed herein is also encompassed. Hybridization of such sequences may be carried out under conditions of reduced stringency or even stringent conditions (e.g., stringent conditions as represented by a wash stringency of 0.3M NaCl, 0.03M sodium citrate, 0.1% SDS at 60° C. or even 70° C.) to nucleic acid encoding Factor IX protein disclosed herein in a standard in situ hybridization assay. See, e.g., Sambrook et al., Molecular Cloning, A Laboratory Manual (2d Ed. 1989) Cold Spring Harbor Laboratory).

The FIX variants produced according to the invention may be expressed in transgenic animals by known methods. See for Example U.S. Pat. No. 6,344,596, which is incorporated herein by reference in its entirety. In brief, transgenic animals may include but are not limited to farm animals (e.g., pigs, goats, sheep, cows, horses, rabbits and the like) rodents (such as mice, rats and guinea pigs), and domestic pets (for example, cats and dogs). Livestock animals such as pigs, sheep, goats and cows, are particularly preferred in some embodiments.

The transgenic animal of this invention is produced by introducing into a single cell embryo an appropriate polynucleotide that encodes a human Factor IX variant of this invention in a manner such that the polynucleotide is stably integrated into the DNA of germ line cells of the mature animal, and is inherited in normal Mendelian fashion. The transgenic animal of this invention would have a phenotype of producing the FIX variant in body fluids and/or tissues. The FIX variant would be removed from these fluids and/or tissues and processed, for example for therapeutic use. (See, e.g., Clark et al. “Expression of human anti-hemophilic factor IX in the milk of transgenic sheep” Bio/Technology 7:487-492 (1989); Van Cott et al. “Haemophilic factors produced by transgenic livestock: abundance can enable alternative therapies worldwide” Haemophilia 10(4):70-77 (2004), the entire contents of which are incorporated by reference herein).

DNA molecules can be introduced into embryos by a variety of means including but not limited to microinjection, calcium phosphate mediated precipitation, liposome fusion, or retroviral infection of totipotent or pluripotent stem cells. The transformed cells can then be introduced into embryos and incorporated therein to form transgenic animals. Methods of making transgenic animals are described, for example, in Transgenic Animal Generation and Use by L. M. Houdebine, Harwood Academic Press, 1997. Transgenic animals also can be generated using methods of nuclear transfer or cloning using embryonic or adult cell lines as described for example in Campbell et al., Nature 380:64-66 (1996) and Wilmut et al., Nature 385:810-813 (1997). Further a technique utilizing cytoplasmic injection of DNA can be used as described in U.S. Pat. No. 5,523,222.

Factor IX-producing transgenic animals can be obtained by introducing a chimeric construct comprising Factor IX-encoding sequences. Methods for obtaining transgenic animals are well-known. See, for example, Hogan et al., MANIPULATING THE MOUSE EMBRYO, (Cold Spring Harbor Press 1986); Krimpenfort et al., Bio/Technology 9:88 (1991); Palmiter et al., Cell 41:343 (1985), Kraemer et al., GENETIC MANIPULATION OF THE EARLY MAMMALIAN EMBRYO, (Cold Spring Harbor Laboratory Press 1985); Hammer et al., Nature 315:680 (1985); Wagner et al., U.S. Pat. No. 5,175,385; Krimpenfort et al., U.S. Pat. No. 5,175,384, Janne et al., Ann. Med. 24:273 (1992), Brem et al., Chim. Oggi. 11:21 (1993), Clark et al., U.S. Pat. No. 5,476,995, all incorporated by reference herein in their entireties.

In some embodiments, cis-acting regulatory regions may be used that are “active” in mammary tissue in that the promoters are more active in mammary tissue than in other tissues under physiological conditions where milk is synthesized. Such promoters include but are not limited to the short and long whey acidic protein (WAP), short and long α, β and κ casein, α-lactalbumin and β-lactoglobulin (“BLG”) promoters. Signal sequences can also be used in accordance with this invention that direct the secretion of expressed proteins into other body fluids, particularly blood and urine. Examples of such sequences include the signal peptides of secreted coagulation factors including signal peptides of Factor IX, protein C, and tissue-type plasminogen activator.

Among the useful sequences that regulate transcription, in addition to the promoters discussed above, are enhancers, splice signals, transcription termination signals, polyadenylation sites, buffering sequences, RNA processing sequences and other sequences which regulate the expression of transgenes.

Preferably, the expression system or construct includes a 3′ untranslated region downstream of the nucleotide sequence encoding the desired recombinant protein. This region can increase expression of the transgene. Among the 3′ untranslated regions useful in this regard are sequences that provide a poly A signal.

Suitable heterologous 3′-untranslated sequences can be derived, for example, from the SV40 small t antigen, the casein 3′ untranslated region, or other 3′ untranslated sequences well known in this art. Ribosome binding sites are also important in increasing the efficiency of expression of FIX. Likewise, sequences that regulate the post-translational modification of FIX are useful in the invention.

Factor IX coding sequences, along with vectors and host cells for the expression thereof, are disclosed in European Patent App. 373012, European Patent App. 251874, PCT Patent Appl. 8505376, PCT Patent Appln. 8505125, European Patent Appln. 162782, and PCT Patent Appln. 8400560, all of which are incorporated by reference herein in their entireties.

Variants in FIX proteins having additional glycosylation sites may be produced by recombinant methods such as site-directed mutagenesis using PCR. Alternatively, the Factor IX variant may be chemically synthesized to prepare a Factor IX protein with one or more additional glycosylation sites.

### EXAMPLES

### Example 1

### Addition of One Glycosylation Site into Human FIX Amino Acid Sequence

A variant of human FIX having one additional glycosylation site in the activation peptide was produced in CHO cells. This variant is stable, has normal activity and an increased half life as compared with wild type recombinant human FIX.

Vectors. FIX-pDEF38 CHEF-1 promoter-containing vector from ICOS was used to express nucleic acid encoding wild type recombinant human FIX or a variant of recombinant human FIX.

Variant FIX. The variant human FIX prepared for these experiments comprises nine extra amino acids containing one extra glycosylation site inserted into the activation peptide (SEQ ID NO:3; FIG. 1, line 3). The sequence of the activation peptide of this variant with the nine added amino acids bolded is AETVFPDVDYVNSTEAETILDNITDGAILNNITQSTQSFNDFTR (SEQ ID NO:133), which shows amino acid 146 at the N terminus and amino acid 181 at the C terminus, with numbering based on the mature human FIX amino acid sequence of SEQ ID NO:33.

Transfection of CHO DG44 cells. Cells are seeded at a density of 3×10⁵ cells/mL in a 125 mL shaker flask containing 15 mL of growth medium and incubated at 37° C. On day 3, cell density should be ˜1×10⁶ cells/mL. DNA-LIPOFECTAMINE 2000 CD complexes are prepared by diluting 20 ug of DNA into 650 ul of OPTIPRO SFM, mixing gently and incubating for 5 minutes at room temperature (RT). LIPOFECTAMINE 2000 CD is mixed gently before use and diluted by putting 45 ul in 650 ul of OPTIPRO SFM, mixing gently and incubating at RT for 5 minutes. After the incubation, the diluted DNA and diluted LIPOFECTAMINE 2000 CD are combined, mixed gently and incubated for 30-45 minutes at RT to allow the DNA-LIPOFECTAMINE 2000 CD complexes to form. After incubation, DNA-LIPOFECTAMINE 2000 CD complexes are added into the shaker flask. After 48 hrs, the cells are spun down and the medium is changed with 30 ml CD OptiCHO™ Medium (Invitrogen. Cat. 12681-011). The medium is changed every 2-3 days to obtain stably transfected cells.

Selection of FIX-expressing cells. Because the dhfr gene is inactivated in DG44 cells, the dhfr gene (Egrie J C, Browne J K. “Development and characterization of novel erythropoiesis stimulating protein (NESP)” Nephrol Dial Transplant. 2001; 16 Suppl 3:3-13) was used as a selection marker. The stably expressing dhfr positive DG44 cells do not require HT for cell growth and can be grown in CD CHO medium.

Purification of hFIX variant proteins from media collected from mixed CHO DG44 cell transfectants and 293 cell clones¹. The purification of rhFIX variant proteins was as follows. EDTA (200 mM, pH 7.4) and benzamidine (1M solution) were added to the crude culture medium to a final concentration of 4 mM and 5 mM, respectively. The culture medium containing the rhFIX variant proteins was mixed with a Q sepharose anion exchange resin at 4° C. The Q sepharose resin was pre-equilibrated with 20 mM Tris, pH 7.4, 0.15M NaCl, 2 mM EDTA, 2 mM benzamidine. The column was washed with 1 L equilibrating buffer and then washed with 200 ml equilibrating buffer without the EDTA. The rhFIX variant protein was eluted with 20 mM Tris, pH 7.4, 0.15M NaCl, 10 mM CaCl₂.

FIX activity. Functional activity of the variant recombinant human FIX was determined by incubating 100 μl human FIX-deficient plasma with 100 μl automated activated partial thromboplastin time (aPTT) reagent (Trinity biotech USA), and 20 μl of test sample diluted with 80 μl Owren-Koller buffer for 3 min at 37° C. To start the reaction, 100 μl of 25 mM CaCl₂ was added, and time to clot formation was measured by eye. The clotting activity of normal pooled human plasma was deemed 100%. FIX-specific activity was calculated by dividing the clotting activity by the total amount of FIX protein as determined by immunoassay and is expressed as units per milligram. The specific activity 116 units per mg for wild type FIX and 104 units per mg for FIX with one extra glycosylation site.

FIX size. An obvious increase in the size of the purified FIX with one extra glycosylation site, as compared to purified plasma FIX and purified wild type recombinant FIX made in CHO cells, was detected by polyacrylamide gel electrophoresis. Upon enzymatic removal of the sugars, the variant FIX migrates approximately with the similarly treated wild type recombinant FIX.

Half life. The half life measurement was done by injecting eight hemophilia B mice with the variant FIX with one extra glycosylation site and injecting eight different hemophilia B mice with wild-type recombinant FIX. One hundred units of FIX/kg was injected into the hemophilia B mice of each group. After injection, the amount of FIX remaining in the circulation was determined at 15 minutes, 4 hr, 12 hr, 24 hr, and 48 hr. The amount of FIX remaining in the circulation was measured by ELISA using wild type FIX as a standard. Antibodies for the ELISA were obtained from Affinity Biologicals (Product numbers SAFIX-AP SAFIX-APHRP). The curve was fit to one exponential decay.

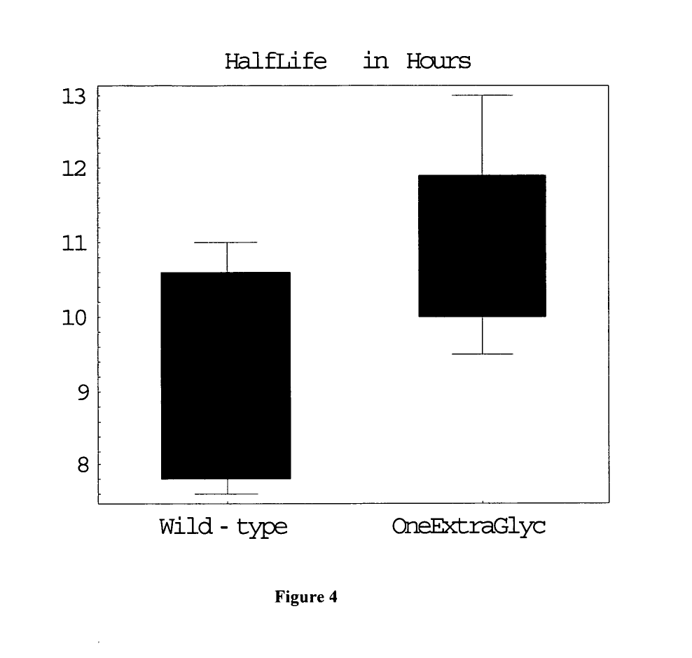

The variant FIX with one extra glycosylation site exhibited a longer half life (about 1.5 hour), as shown in FIG. 4. Further analysis of this variant will be carried out to determine whether there is complete sialylation of this FIX protein, as incomplete sialylation can result in a shorter half life, as reported by Griffith². Assays to determine the degree of sialylation are well known in the art (See, e.g., Anumula and Dhume “High resolution and high sensitivity methods for oligosaccharide mapping and characterization by normal phase high performance liquid chromatography following derivatization with highly fluorescent anthranilic acid” Glycobiology 8:685-694 (1998); Liu et al. “Human plasma N-glycoproteome analysis by immunoaffinity subtraction, hydrazide chemistry, and mass spectrometry” J. Proteome Res. 4(6):2070-2080 (2005)), the entire contents of each of which are incorporated by reference herein).

The half-life of a proteins can be influenced by many factors. Simple size has a major effect on whether a circulating protein is maintained in the circulation or is distributed throughout the body. In addition, specific binding sites may remove proteins from circulation. It is known that plasma proteins that are under-sialylated have exposed GlcNAc and Gal residues that are removed from circulation by the asialoglycoprotein liver receptors³⁻⁵. There is a family of 18 different sialyl transferase enzymes that are differentially expressed in mammalian tissues⁶. In humans, the N-glycosylated N-terminal galactoses are usually terminated by α(2,6) sialic acid. CHO or BHK cells produce FIX in which the N-glycosylated terminal galactose is capped by α(2,3)-sialylated galactose. However FIX produced in 293 cells is capped by sialic acid on α(2,6)-galactose. Under-sialylation could easily lead to an increased clearance rate from the circulation and mask the expected half-life increase resulting from the extra glycosylation. Under-sialylation may be improved either in vitro (by enzymatic ally adding sialic acids³) or in cell culture by either adding sialylation enzymes to the cells expressing recombinant FIX or by manipulating culture conditions to increase sialylation⁷⁻¹⁰. It has been shown also that transfect ion of CHO cells with the gene for Gal(β1-4)GlcNAc-R α(2,6)-sialyltransferase overwhelms the endogenous sialylation enzymes and results in the production of recombinant proteins bearing terminal α-(2,6)-sialyl-galactose as the major modification¹¹.

This study demonstrates that amino acid residues can be inserted into the activation peptide of human factor IX without materially affecting its clotting time and that these insertions have no deleterious effect on the production of human factor IX. These studies further demonstrate that any amino acid sequence can be incorporated into the activation peptide of factor IX, provided it does not contain any sequences that would loop back into the FIX protein itself and disrupt structure, as would be readily identified by one of ordinary skill in the art using well known techniques. Furthermore, amino acid sequences could be incorporated that allow chemical addition of specific sites for adding compounds such as polyethylene glycol to further extend half-life. Such sequences could be produced and tested according to standard protocols using routine experimentation.

### Example 2

### Variant Human FIX with No New Glycosylation Sites Introduced

As a demonstration that a very different sequence can also be inserted into the activation peptide of human FIX without adversely affecting the FIX molecule the following amino acid sequence FLNCCPGCCMEP (SEQ ID NO:134) was inserted into the activation peptide between amino acids 161 and 162 (numbering is based on the mature FIX amino acid sequence as shown in SEQ ID NO:33). This recombinant protein was analyzed according to the method set forth in Example 1 above and was shown to have the same functionality as wild type recombinant human FIX.

It will be understood by those of skill in the art that numerous and various modifications can be made without departing from the spirit of the present invention. Therefore, it should be clearly understood that the forms of the present invention are illustrative only and are not intended to limit the scope of the present invention.

All publications, patent applications, patents, patent publications, sequences identified by GenBank® database accession numbers and other references cited herein are incorporated by reference in their entireties for the teachings relevant to the sentence and/or paragraph in which the reference is presented.

The invention is defined by the following claims, with equivalents of the claims to be included therein.

### REFERENCES FOR EXAMPLE 1

1. Yan S C, Razzano P, Chao Y B, et al. Characterization and novel purification of recombinant human protein C from three mammalian cell lines. Biotechnology (N Y). 1990; 8:655-661.
2. Griffith M J, Monroe D M, van Cott D E, et al. N-GLYCAN SIALYLATION IS IMPORTANT FOR IN VIVO RECOVERY OF RECOMBINANT FACTOR IX J Thromb Haemost. 2007; 5:P-M-043.
3. Raju T S, Briggs J B, Chamow S M, Winkler M E, Jones A J. Glycoengineering of therapeutic glycoproteins: in vitro galactosylation and sialylation of glycoproteins with terminal N-acetylglucosamine and galactose residues. Biochemistry. 2001; 40:8868-8876.
4. Joziasse D H, Lee R T, Lee Y C, et al. alpha3-galactosylated glycoproteins can bind to the hepatic asialoglycoprotein receptor. Eur J. Biochem. 2000; 267:6501-6508.
5. Van den Nieuwenhof I M, Koistinen H, Easton R L, et al. Recombinant glycodelin carrying the same type of glycan structures as contraceptive glycodelin-A can be produced in human kidney 293 cells but not in Chinese hamster ovary cells. Eur J Biochem. 2000; 267:4753-4762.
6. Takashima S, Kurosawa N, Tachida Y, Inoue M, Tsuji S. Comparative analysis of the genomic structures and promoter activities of mouse Siaalpha2,3Galbeta1,3GalNAc GalNAcalpha2,6-sialyltransferase genes (ST6Ga1NAc III and IV): characterization of their Spl binding sites. J Biochem (Tokyo). 2000; 127:399-409.
7. Chee Furng Wong D, Tin Kam Wong K, Tang Goh L, Kiat Heng C, Gek Sim Yap M. Impact of dynamic online fed-batch strategies on metabolism, productivity and N-glycosylation quality in CHO cell cultures. Biotechnol Bioeng. 2005; 89:164-177.
8. Chen P, Harcum S W. Effects of amino acid additions on ammonium stressed CHO cells. J Biotechnol. 2005; 117:277-286.
9. Chen P, Harcum S W. Effects of elevated ammonium on glycosylation gene expression in CHO cells. Metab Eng. 2006; 8:123-132.
10. Nam J H, Zhang F, Ermonval M, Linhardt R J, Sharfstein S T. The effects of culture conditions on the glycosylation of secreted human placental alkaline phosphatase produced in Chinese hamster ovary cells. Biotechnol Bioeng. 2008; 100:1178-1192.
11. Grabenhorst E, Hoffmann A, Nimtz M, Zettlmeissl G, Conradt H S. Construction of stable BHK-21 cells coexpressing human secretory glycoproteins and human Gal(beta 1-4)GlcNAc-R alpha 2,6-sialyltransferase alpha 2,6-linked NeuAc is preferentially attached to the Gal(beta 1-4)GlcNAc(beta 1-2)Man(alpha 1-3)-branch of diantennary oligosaccharides from secreted recombinant beta-trace protein. Eur J Biochem. 1995; 232:718-725.

TABLE 1

| Amino Acids | One-Letter Symbol | Common Abbreviation |
|---|---|---|
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

TABLE 2
Solvent Accessible Surface Area (ASA) calculation parameters:
Sphere radius: 1.4
Burial threshold: 0.25

| | | | | | | | SC |
|---|---|---|---|---|---|---|---|
| | | Bck | S.Chn | Total | SC Ref. | Percent | Class |
| TYR | 1 | 0.27 | 125.59 | 125.86 | 232.25 | 54.08 | E |
| ASN | 2 | 7.49 | 64.20 | 71.70 | 152.56 | 42.08 | E |
| SER | 3 | 31.85 | 35.49 | 67.34 | 104.76 | 33.88 | E |
| GLY | 4 | 35.78 | 13.41 | 49.18 | 30.80 | 43.53 | E |
| LYS | 5 | 16.74 | 158.41 | 175.15 | 217.63 | 72.79 | E |
| LEU | 6 | 8.28 | 135.99 | 144.28 | 185.25 | 73.41 | E |
| GLU | 7 | 8.50 | 21.04 | 29.54 | 168.29 | 12.50 | B |
| GLU | 8 | 30.76 | 44.77 | 75.53 | 168.29 | 26.60 | E |
| PHE | 9 | 24.15 | 164.83 | 188.98 | 220.07 | 74.90 | E |
| VAL | 10 | 13.07 | 51.73 | 64.80 | 157.34 | 32.88 | E |
| GLN | 11 | 31.24 | 149.22 | 180.46 | 183.52 | 81.31 | E |
| GLY | 12 | 24.92 | 3.98 | 28.90 | 30.80 | 12.91 | B |
| ASN | 13 | 6.63 | 59.16 | 65.79 | 152.56 | 38.78 | E |
| LEU | 14 | 11.27 | 83.39 | 94.66 | 185.25 | 45.02 | E |
| GLU | 15 | 2.60 | 116.90 | 119.50 | 168.29 | 69.47 | E |
| ARG | 16 | 0.86 | 88.62 | 89.49 | 249.57 | 35.51 | E |
| GLU | 17 | 0.00 | 18.71 | 18.71 | 168.29 | 11.12 | B |
| CYS | 18 | 8.83 | 5.31 | 14.14 | 129.22 | 4.11 | B |
| MET | 19 | 18.21 | 138.84 | 157.06 | 195.71 | 70.94 | E |
| GLU | 20 | 39.22 | 46.20 | 85.41 | 168.29 | 27.45 | E |
| GLU | 21 | 13.25 | 19.74 | 32.98 | 168.29 | 11.73 | B |
| LYS | 22 | 14.47 | 176.83 | 191.30 | 217.63 | 81.25 | E |
| CYS | 23 | 16.54 | 4.14 | 20.68 | 129.22 | 3.20 | B |
| SER | 24 | 7.99 | 31.53 | 39.52 | 104.76 | 30.10 | E |
| PHE | 25 | 5.59 | 64.06 | 69.65 | 220.07 | 29.11 | E |
| GLU | 26 | 5.05 | 82.90 | 87.96 | 168.29 | 49.26 | E |
| GLU | 27 | 1.52 | 7.49 | 9.00 | 168.29 | 4.45 | B |
| ALA | 28 | 4.70 | 3.15 | 7.85 | 86.68 | 3.63 | B |
| ARG | 29 | 4.39 | 118.91 | 123.30 | 249.57 | 47.64 | E |
| GLU | 30 | 32.80 | 52.38 | 85.18 | 168.29 | 31.13 | E |
| VAL | 31 | 26.79 | 57.63 | 84.42 | 157.34 | 36.63 | E |
| PHE | 32 | 15.66 | 55.53 | 71.18 | 220.07 | 25.23 | E |
| GLU | 33 | 38.84 | 97.20 | 136.05 | 168.29 | 57.76 | E |
| ASN | 34 | 8.70 | 57.51 | 66.21 | 152.56 | 37.70 | E |
| THR | 35 | 6.61 | 62.61 | 69.22 | 141.80 | 44.15 | E |
| GLU | 36 | 4.28 | 98.00 | 102.28 | 168.29 | 58.23 | E |
| ARG | 37 | 2.80 | 119.95 | 122.76 | 249.57 | 48.06 | E |
| THR | 38 | 0.00 | 0.00 | 0.00 | 141.80 | 0.00 | B |
| THR | 39 | 0.00 | 54.41 | 54.41 | 141.80 | 38.37 | E |
| GLU | 40 | 10.13 | 57.40 | 67.54 | 168.29 | 34.11 | E |
| PHE | 41 | 8.14 | 42.37 | 50.50 | 220.07 | 19.25 | B |
| TRP | 42 | 0.00 | 61.69 | 61.69 | 267.74 | 23.04 | B |
| LYS | 43 | 10.33 | 119.92 | 130.25 | 217.63 | 55.10 | E |
| GLN | 44 | 4.98 | 133.39 | 138.37 | 183.52 | 72.69 | E |
| TYR | 45 | 25.77 | 75.81 | 101.58 | 232.25 | 32.64 | E |
| VAL | 46 | 8.23 | 65.11 | 73.35 | 157.34 | 41.38 | E |
| ASP | 47 | 20.51 | 55.77 | 76.27 | 135.82 | 41.06 | E |
| GLY | 48 | 10.54 | 2.70 | 13.24 | 30.80 | 8.77 | B |
| ASP | 49 | 0.58 | 35.16 | 35.73 | 135.82 | 25.89 | E |
| GLN | 50 | 6.81 | 104.99 | 111.80 | 183.52 | 57.21 | E |
| CYS | 51 | 3.97 | 22.00 | 25.97 | 129.22 | 17.03 | B |
| GLU | 52 | 31.64 | 139.49 | 171.12 | 168.29 | 82.89 | E |
| SER | 53 | 37.04 | 48.46 | 85.50 | 104.76 | 46.26 | E |
| ASN | 54 | 16.68 | 102.95 | 119.64 | 152.56 | 67.48 | E |
| PRO | 55 | 19.36 | 7.93 | 27.29 | 158.05 | 5.02 | B |
| CYS | 56 | 12.01 | 2.30 | 14.30 | 129.22 | 1.78 | B |
| LEU | 57 | 6.35 | 89.67 | 96.02 | 185.25 | 48.40 | E |
| ASN | 58 | 49.16 | 42.59 | 91.75 | 152.56 | 27.92 | E |
| GLY | 59 | 48.82 | 13.15 | 61.97 | 30.80 | 42.69 | E |
| GLY | 60 | 3.57 | 0.40 | 3.97 | 30.80 | 1.30 | B |
| SER | 61 | 11.00 | 85.41 | 96.41 | 104.76 | 81.53 | E |
| CYS | 62 | 17.13 | 1.83 | 18.96 | 129.22 | 1.42 | B |
| LYS | 63 | 7.94 | 139.66 | 147.60 | 217.63 | 64.17 | E |
| ASP | 64 | 15.09 | 57.49 | 72.58 | 135.82 | 42.32 | E |
| ASP | 65 | 7.60 | 43.64 | 51.24 | 135.82 | 32.13 | E |
| ILE | 66 | 30.84 | 144.89 | 175.73 | 187.72 | 77.18 | E |
| ASN | 67 | 13.32 | 122.54 | 135.85 | 152.56 | 80.32 | E |
| SER | 68 | 3.81 | 36.41 | 40.23 | 104.76 | 34.76 | E |
| TYR | 69 | 14.11 | 26.83 | 40.93 | 232.25 | 11.55 | B |
| GLU | 70 | 5.59 | 73.21 | 78.80 | 168.29 | 43.50 | E |
| CYS | 71 | 6.68 | 0.67 | 7.35 | 129.22 | 0.52 | B |
| TRP | 72 | 7.49 | 115.47 | 122.97 | 267.74 | 43.13 | E |
| CYS | 73 | 1.76 | 7.99 | 9.75 | 129.22 | 6.19 | B |
| PRO | 74 | 6.36 | 67.68 | 74.04 | 158.05 | 42.82 | E |
| PHE | 75 | 7.24 | 67.64 | 74.88 | 220.07 | 30.73 | E |
| GLY | 76 | 5.84 | 8.63 | 14.47 | 30.80 | 28.03 | E |
| PHE | 77 | 1.57 | 60.84 | 62.41 | 220.07 | 27.65 | E |
| GLU | 78 | 4.54 | 42.38 | 46.92 | 168.29 | 25.18 | E |
| GLY | 79 | 33.91 | 4.27 | 38.18 | 30.80 | 13.86 | B |
| LYS | 80 | 28.88 | 148.45 | 177.33 | 217.63 | 68.21 | E |
| ASN | 81 | 0.00 | 33.47 | 33.47 | 152.56 | 21.94 | B |
| CYS | 82 | 0.00 | 0.23 | 0.23 | 129.22 | 0.18 | B |
| GLU | 83 | 19.73 | 71.03 | 90.77 | 168.29 | 42.21 | E |
| LEU | 84 | 1.61 | 106.59 | 108.20 | 185.25 | 57.54 | E |
| ASP | 85 | 5.18 | 78.25 | 83.43 | 135.82 | 57.61 | E |
| VAL | 86 | 0.61 | 0.52 | 1.13 | 157.34 | 0.33 | B |
| THR | 87 | 0.56 | 59.85 | 60.41 | 141.80 | 42.21 | E |
| CYS | 88 | 12.82 | 25.94 | 38.77 | 129.22 | 20.08 | B |
| ASN | 89 | 32.29 | 109.18 | 141.47 | 152.56 | 71.56 | E |
| ILE | 90 | 9.42 | 71.41 | 80.83 | 187.72 | 38.04 | E |
| LYS | 91 | 31.54 | 47.20 | 78.74 | 217.63 | 21.69 | B |
| ASN | 92 | 14.48 | 28.84 | 43.32 | 152.56 | 18.90 | B |
| GLY | 93 | 0.00 | 0.00 | 0.00 | 30.80 | 0.00 | B |
| ARG | 94 | 4.85 | 114.06 | 118.90 | 249.57 | 45.70 | E |
| CYS | 95 | 0.00 | 0.00 | 0.00 | 129.22 | 0.00 | B |
| GLU | 96 | 0.10 | 40.37 | 40.48 | 168.29 | 23.99 | B |
| GLN | 97 | 0.00 | 0.00 | 0.00 | 183.52 | 0.00 | B |
| PHE | 98 | 0.00 | 38.92 | 38.92 | 220.07 | 17.68 | B |
| CYS | 99 | 0.93 | 1.21 | 2.13 | 129.22 | 0.93 | B |
| LYS | 100 | 1.98 | 161.78 | 163.76 | 217.63 | 74.34 | E |
| ASN | 101 | 26.90 | 32.19 | 59.09 | 152.56 | 21.10 | B |
| SER | 102 | 17.07 | 15.09 | 32.17 | 104.76 | 14.41 | B |
| ALA | 103 | 53.92 | 73.38 | 127.30 | 86.68 | 84.65 | E |
| ASP | 104 | 29.80 | 71.30 | 101.10 | 135.82 | 52.49 | E |
| ASN | 105 | 5.98 | 116.68 | 122.66 | 152.56 | 76.48 | E |
| LYS | 106 | 6.97 | 78.17 | 85.14 | 217.63 | 35.92 | E |
| VAL | 107 | 2.15 | 2.48 | 4.63 | 157.34 | 1.57 | B |
| VAL | 108 | 4.70 | 79.48 | 84.18 | 157.34 | 50.52 | E |
| CYS | 109 | 6.11 | 0.74 | 6.84 | 129.22 | 0.57 | B |
| SER | 110 | 3.10 | 48.75 | 51.85 | 104.76 | 46.53 | E |
| CYS | 111 | 19.48 | 3.24 | 22.72 | 129.22 | 2.51 | B |
| THR | 112 | 1.32 | 1.85 | 3.17 | 141.80 | 1.31 | B |
| GLU | 113 | 16.54 | 86.13 | 102.67 | 168.29 | 51.18 | E |
| GLY | 114 | 27.26 | 2.40 | 29.66 | 30.80 | 7.79 | B |
| TYR | 115 | 19.30 | 0.93 | 20.23 | 232.25 | 0.40 | B |
| ARG | 116 | 7.93 | 125.32 | 133.25 | 249.57 | 50.22 | E |
| LEU | 117 | 24.16 | 36.08 | 60.24 | 185.25 | 19.48 | B |
| ALA | 118 | 6.67 | 13.31 | 19.98 | 86.68 | 15.36 | B |
| GLU | 119 | 24.69 | 145.15 | 169.84 | 168.29 | 86.25 | E |
| ASN | 120 | 31.18 | 62.55 | 93.73 | 152.56 | 41.00 | E |
| GLN | 121 | 3.07 | 82.40 | 85.46 | 183.52 | 44.90 | E |
| LYS | 122 | 0.00 | 38.14 | 38.14 | 217.63 | 17.53 | B |
| SER | 123 | 0.12 | 38.91 | 39.03 | 104.76 | 37.14 | E |
| CYS | 124 | 1.30 | 0.00 | 1.30 | 129.22 | 0.00 | B |
| GLU | 125 | 5.57 | 83.86 | 89.42 | 168.29 | 49.83 | E |
| PRO | 126 | 10.45 | 60.74 | 71.18 | 158.05 | 38.43 | E |
| ALA | 127 | 27.45 | 24.33 | 51.79 | 86.68 | 28.07 | E |
| VAL | 128 | 9.37 | 73.68 | 83.05 | 157.34 | 46.83 | E |
| PRO | 129 | 27.18 | 94.73 | 121.91 | 158.05 | 59.94 | E |
| PHE | 130 | 9.31 | 54.32 | 63.63 | 220.07 | 24.68 | B |
| PRO | 131 | 9.64 | 8.80 | 18.44 | 158.05 | 5.57 | B |
| CYS | 132 | 3.95 | 17.82 | 21.77 | 129.22 | 13.79 | B |
| GLY | 133 | 0.20 | 0.00 | 0.20 | 30.80 | 0.00 | B |
| ARG | 134 | 1.28 | 63.41 | 64.69 | 249.57 | 25.41 | E |
| VAL | 135 | 3.16 | 22.36 | 25.51 | 157.34 | 14.21 | B |
| SER | 136 | 4.80 | 9.07 | 13.87 | 104.76 | 8.66 | B |
| VAL | 137 | 6.47 | 1.77 | 8.24 | 157.34 | 1.13 | B |
| SER | 138 | 0.97 | 46.05 | 47.03 | 104.76 | 43.96 | E |
| GLN | 139 | 37.96 | 108.90 | 146.86 | 183.52 | 59.34 | E |
| THR | 140 | 23.25 | 93.55 | 116.79 | 141.80 | 65.97 | E |
| SER | 141 | 10.17 | 0.52 | 10.69 | 104.76 | 0.49 | B |
| LYS | 142 | 9.33 | 172.08 | 181.41 | 217.63 | 79.07 | E |
| LEU | 143 | 16.86 | 120.73 | 137.60 | 185.25 | 65.17 | E |
| THR | 144 | 7.19 | 6.03 | 13.22 | 141.80 | 4.25 | B |
| ARG | 145 | 18.07 | 91.85 | 109.92 | 249.57 | 36.80 | E |
| ALA | 146 | 37.80 | 70.61 | 108.41 | 86.68 | 81.46 | E |
| GLU | 147 | 9.26 | 89.70 | 98.96 | 168.29 | 53.30 | E |
| ALA | 148 | 29.35 | 71.17 | 100.53 | 86.68 | 82.11 | E |
| VAL | 149 | 3.62 | 80.97 | 84.60 | 157.34 | 51.46 | E |
| PHE | 150 | 0.00 | 22.28 | 22.28 | 220.07 | 10.13 | B |
| PRO | 151 | 11.40 | 93.90 | 105.30 | 158.05 | 59.41 | E |
| ASP | 152 | 8.22 | 89.42 | 97.64 | 135.82 | 65.84 | E |
| VAL | 153 | 0.33 | 53.16 | 53.49 | 157.34 | 33.79 | E |
| ASP | 154 | 0.94 | 45.36 | 46.31 | 135.82 | 33.40 | E |
| TYR | 155 | 39.87 | 135.86 | 175.72 | 232.25 | 58.50 | E |
| VAL | 156 | 2.41 | 81.67 | 84.09 | 157.34 | 51.91 | E |
| ASN | 157 | 15.87 | 53.80 | 69.67 | 152.56 | 35.27 | E |
| SER | 158 | 9.86 | 52.72 | 62.58 | 104.76 | 50.33 | E |
| THR | 159 | 37.44 | 87.63 | 125.07 | 141.80 | 61.80 | E |
| GLU | 160 | 19.85 | 122.04 | 141.89 | 168.29 | 72.52 | E |
| ALA | 161 | 21.20 | 10.01 | 31.21 | 86.68 | 11.54 | B |
| GLU | 162 | 15.99 | 96.79 | 112.78 | 168.29 | 57.52 | E |
| THR | 163 | 9.85 | 112.60 | 122.45 | 141.80 | 79.41 | E |
| ILE | 164 | 13.67 | 123.33 | 137.00 | 187.72 | 65.70 | E |
| LEU | 165 | 0.78 | 34.33 | 35.11 | 185.25 | 18.53 | B |
| ASP | 166 | 1.58 | 75.75 | 77.34 | 135.82 | 55.78 | E |
| ASN | 167 | 2.85 | 72.64 | 75.50 | 152.56 | 47.62 | E |
| ILE | 168 | 0.00 | 3.68 | 3.68 | 187.72 | 1.96 | B |
| THR | 169 | 0.19 | 26.20 | 26.39 | 141.80 | 18.48 | B |
| GLN | 170 | 9.30 | 96.68 | 105.98 | 183.52 | 52.68 | E |
| SER | 171 | 6.68 | 5.60 | 12.29 | 104.76 | 5.35 | B |
| THR | 172 | 11.09 | 12.16 | 23.26 | 141.80 | 8.58 | B |
| GLN | 173 | 35.92 | 68.99 | 104.91 | 183.52 | 37.59 | E |
| SER | 174 | 25.99 | 50.08 | 76.07 | 104.76 | 47.80 | E |
| PHE | 175 | 25.20 | 99.76 | 124.96 | 220.07 | 45.33 | E |
| ASN | 176 | 20.49 | 60.35 | 80.83 | 152.56 | 39.56 | E |
| ASP | 177 | 9.36 | 59.18 | 68.54 | 135.82 | 43.57 | E |
| PHE | 178 | 0.03 | 92.07 | 92.10 | 220.07 | 41.84 | E |
| THR | 179 | 0.02 | 23.46 | 23.48 | 141.80 | 16.55 | B |
| ARG | 180 | 0.00 | 106.62 | 106.62 | 249.57 | 42.72 | E |
| VAL | 181 | 0.81 | 51.30 | 52.11 | 157.34 | 32.61 | E |
| VAL | 182 | 19.24 | 109.54 | 128.78 | 157.34 | 69.62 | E |
| GLY | 183 | 24.71 | 13.96 | 38.67 | 30.80 | 45.34 | E |
| GLY | 184 | 8.70 | 0.60 | 9.31 | 30.80 | 1.96 | B |
| GLU | 185 | 0.17 | 12.09 | 12.25 | 168.29 | 7.18 | B |
| ASP | 186 | 14.39 | 65.47 | 79.86 | 135.82 | 48.20 | E |
| ALA | 187 | 4.77 | 0.00 | 4.77 | 86.68 | 0.00 | B |
| LYS | 188 | 1.92 | 14.45 | 16.36 | 217.63 | 6.64 | B |
| PRO | 189 | 0.57 | 6.07 | 6.64 | 158.05 | 3.84 | B |
| GLY | 190 | 8.40 | 0.00 | 8.40 | 30.80 | 0.00 | B |
| GLN | 191 | 3.54 | 30.26 | 33.80 | 183.52 | 16.49 | B |
| PHE | 192 | 7.66 | 6.33 | 13.98 | 220.07 | 2.88 | B |
| PRO | 193 | 4.58 | 0.31 | 4.89 | 158.05 | 0.20 | B |
| TRP | 194 | 0.00 | 2.91 | 2.91 | 267.74 | 1.09 | B |
| GLN | 195 | 4.69 | 0.06 | 4.75 | 183.52 | 0.03 | B |
| VAL | 196 | 0.00 | 0.00 | 0.00 | 157.34 | 0.00 | B |
| VAL | 197 | 0.08 | 16.00 | 16.08 | 157.34 | 10.17 | B |
| LEU | 198 | 0.00 | 0.00 | 0.00 | 185.25 | 0.00 | B |
| ASN | 199 | 0.00 | 61.15 | 61.15 | 152.56 | 40.08 | E |
| GLY | 200 | 6.11 | 0.00 | 6.11 | 30.80 | 0.00 | B |
| LYS | 201 | 6.18 | 73.82 | 80.00 | 217.63 | 33.92 | E |
| VAL | 202 | 50.22 | 117.81 | 168.03 | 157.34 | 74.88 | E |
| ASP | 203 | 8.10 | 60.74 | 68.84 | 135.82 | 44.72 | E |
| ALA | 204 | 4.63 | 30.78 | 35.41 | 86.68 | 35.51 | E |
| PHE | 205 | 10.41 | 13.15 | 23.56 | 220.07 | 5.98 | B |
| CYS | 206 | 0.45 | 0.90 | 1.36 | 129.22 | 0.70 | B |
| GLY | 207 | 0.00 | 0.00 | 0.00 | 30.80 | 0.00 | B |
| GLY | 208 | 0.00 | 0.00 | 0.00 | 30.80 | 0.00 | B |
| SER | 209 | 0.00 | 0.00 | 0.00 | 104.76 | 0.00 | B |
| ILE | 210 | 0.00 | 0.00 | 0.00 | 187.72 | 0.00 | B |
| VAL | 211 | 15.63 | 4.49 | 20.12 | 157.34 | 2.85 | B |
| ASN | 212 | 3.85 | 43.00 | 46.85 | 152.56 | 28.19 | E |
| GLU | 213 | 2.18 | 87.87 | 90.05 | 168.29 | 52.22 | E |
| LYS | 214 | 0.00 | 56.82 | 56.82 | 217.63 | 26.11 | E |
| TRP | 215 | 0.00 | 28.11 | 28.11 | 267.74 | 10.50 | B |
| ILE | 216 | 0.00 | 0.00 | 0.00 | 187.72 | 0.00 | B |
| VAL | 217 | 0.00 | 0.00 | 0.00 | 157.34 | 0.00 | B |
| THR | 218 | 0.00 | 0.00 | 0.00 | 141.80 | 0.00 | B |
| ALA | 219 | 0.00 | 0.00 | 0.00 | 86.68 | 0.00 | B |
| ALA | 220 | 0.72 | 0.00 | 0.72 | 86.68 | 0.00 | B |
| CYS | 222 | 0.15 | 0.00 | 0.15 | 129.22 | 0.00 | B |
| VAL | 223 | 3.08 | 0.33 | 3.40 | 157.34 | 0.21 | B |
| GLU | 224 | 5.42 | 62.67 | 68.09 | 168.29 | 37.24 | E |
| THR | 225 | 55.43 | 85.50 | 140.93 | 141.80 | 60.30 | E |
| GLY | 226 | 23.22 | 15.40 | 38.62 | 30.80 | 49.99 | E |
| VAL | 227 | 14.36 | 32.05 | 46.41 | 157.34 | 20.37 | B |
| LYS | 228 | 21.74 | 169.93 | 191.67 | 217.63 | 78.08 | E |
| ILE | 229 | 1.70 | 0.00 | 1.70 | 187.72 | 0.00 | B |
| THR | 230 | 0.00 | 43.99 | 43.99 | 141.80 | 31.02 | E |
| VAL | 231 | 1.40 | 0.00 | 1.40 | 157.34 | 0.00 | B |
| VAL | 232 | 0.00 | 22.26 | 22.26 | 157.34 | 14.15 | B |
| ALA | 233 | 0.00 | 0.00 | 0.00 | 86.68 | 0.00 | B |
| GLY | 234 | 5.58 | 0.00 | 5.58 | 30.80 | 0.00 | B |
| GLU | 235 | 11.21 | 10.69 | 21.90 | 168.29 | 6.35 | B |
| ASN | 237 | 0.00 | 24.88 | 24.88 | 152.56 | 16.31 | B |
| ILE | 238 | 25.21 | 36.69 | 61.90 | 187.72 | 19.54 | B |
| GLU | 239 | 35.47 | 73.18 | 108.65 | 168.29 | 43.48 | E |
| GLU | 240 | 6.25 | 104.93 | 111.18 | 168.29 | 62.35 | E |
| THR | 241 | 27.21 | 106.54 | 133.75 | 141.80 | 75.13 | E |
| GLU | 242 | 13.37 | 27.36 | 40.73 | 168.29 | 16.26 | B |
| THR | 244 | 7.49 | 10.34 | 17.83 | 141.80 | 7.29 | B |
| GLU | 245 | 14.27 | 29.00 | 43.27 | 168.29 | 17.23 | B |
| GLN | 246 | 4.49 | 22.72 | 27.22 | 183.52 | 12.38 | B |
| LYS | 247 | 12.94 | 163.01 | 175.95 | 217.63 | 74.90 | E |
| ARG | 248 | 7.70 | 34.16 | 41.85 | 249.57 | 13.69 | B |
| ASN | 249 | 4.99 | 64.87 | 69.87 | 152.56 | 42.52 | E |
| VAL | 250 | 8.42 | 0.00 | 8.42 | 157.34 | 0.00 | B |
| ILE | 251 | 32.72 | 66.80 | 99.51 | 187.72 | 35.58 | E |
| ARG | 252 | 4.11 | 113.04 | 117.14 | 249.57 | 45.29 | E |
| ILE | 253 | 2.07 | 32.52 | 34.58 | 187.72 | 17.32 | B |
| ILE | 254 | 5.06 | 124.37 | 129.43 | 187.72 | 66.25 | E |
| PRO | 255 | 1.25 | 22.02 | 23.27 | 158.05 | 13.94 | B |
| ASN | 258 | 3.97 | 73.90 | 77.87 | 152.56 | 48.44 | E |
| TYR | 259 | 8.25 | 19.59 | 27.84 | 232.25 | 8.43 | B |
| ASN | 260 | 36.09 | 85.16 | 121.25 | 152.56 | 55.82 | E |
| ALA | 261 | 27.50 | 23.02 | 50.52 | 86.68 | 26.55 | E |
| ALA | 262 | 54.08 | 69.46 | 123.54 | 86.68 | 80.14 | E |
| ILE | 263 | 13.33 | 75.87 | 89.19 | 187.72 | 40.41 | E |
| ASN | 264 | 16.68 | 24.65 | 41.33 | 152.56 | 16.16 | B |
| LYS | 265 | 4.07 | 126.06 | 130.12 | 217.63 | 57.92 | E |
| TYR | 266 | 0.38 | 51.66 | 52.04 | 232.25 | 22.24 | B |
| ASN | 267 | 1.12 | 9.95 | 11.07 | 152.56 | 6.52 | B |
| ASP | 269 | 0.00 | 3.49 | 3.49 | 135.82 | 2.57 | B |
| ILE | 270 | 0.00 | 0.00 | 0.00 | 187.72 | 0.00 | B |
| ALA | 271 | 0.00 | 0.00 | 0.00 | 86.68 | 0.00 | B |
| LEU | 272 | 1.11 | 0.07 | 1.18 | 185.25 | 0.04 | B |
| LEU | 273 | 0.34 | 0.00 | 0.34 | 185.25 | 0.00 | B |
| GLU | 274 | 0.00 | 18.28 | 18.28 | 168.29 | 10.86 | B |
| LEU | 275 | 0.00 | 0.00 | 0.00 | 185.25 | 0.00 | B |
| ASP | 276 | 16.49 | 57.20 | 73.69 | 135.82 | 42.11 | E |
| GLU | 277 | 2.61 | 96.37 | 98.99 | 168.29 | 57.27 | E |
| PRO | 278 | 2.44 | 69.22 | 71.66 | 158.05 | 43.80 | E |
| LEU | 279 | 3.48 | 0.00 | 3.48 | 185.25 | 0.00 | B |
| VAL | 280 | 4.62 | 111.51 | 116.13 | 157.34 | 70.87 | E |
| LEU | 281 | 15.17 | 28.43 | 43.60 | 185.25 | 15.35 | B |
| ASN | 282 | 0.29 | 40.69 | 40.98 | 152.56 | 26.67 | E |
| SER | 283 | 2.66 | 27.61 | 30.27 | 104.76 | 26.35 | E |
| TYR | 284 | 1.12 | 51.82 | 52.94 | 232.25 | 22.31 | B |
| VAL | 285 | 0.00 | 0.00 | 0.00 | 157.34 | 0.00 | B |
| THR | 286 | 0.00 | 0.95 | 0.95 | 141.80 | 0.67 | B |
| PRO | 287 | 0.00 | 5.93 | 5.93 | 158.05 | 3.75 | B |
| ILE | 288 | 2.87 | 0.12 | 2.98 | 187.72 | 0.06 | B |
| CYS | 289 | 1.72 | 0.15 | 1.87 | 129.22 | 0.11 | B |
| ILE | 290 | 17.34 | 23.72 | 41.07 | 187.72 | 12.64 | B |
| ALA | 291 | 14.96 | 0.76 | 15.71 | 86.68 | 0.88 | B |
| ASP | 292 | 17.82 | 71.99 | 89.81 | 135.82 | 53.01 | E |
| LYS | 293 | 0.30 | 128.24 | 128.55 | 217.63 | 58.93 | E |
| GLU | 294 | 2.05 | 120.77 | 122.82 | 168.29 | 71.76 | E |
| TYR | 295 | 0.04 | 41.29 | 41.33 | 232.25 | 17.78 | B |
| THR | 296 | 0.00 | 0.61 | 0.61 | 141.80 | 0.43 | B |
| ASN | 297 | 0.00 | 62.71 | 62.71 | 152.56 | 41.11 | E |
| ILE | 298 | 4.83 | 94.49 | 99.32 | 187.72 | 50.34 | E |
| PHE | 299 | 3.42 | 14.54 | 17.97 | 220.07 | 6.61 | B |
| LEU | 300 | 2.16 | 9.56 | 11.72 | 185.25 | 5.16 | B |
| LYS | 301 | 25.40 | 142.97 | 168.37 | 217.63 | 65.69 | E |
| PHE | 302 | 27.30 | 158.84 | 186.14 | 220.07 | 72.18 | E |
| GLY | 303 | 8.18 | 18.77 | 26.95 | 30.80 | 60.93 | E |
| SER | 304 | 9.64 | 28.10 | 37.74 | 104.76 | 26.83 | E |
| GLY | 305 | 4.68 | 0.00 | 4.68 | 30.80 | 0.00 | B |
| TYR | 306 | 0.02 | 72.96 | 72.98 | 232.25 | 31.41 | E |
| VAL | 307 | 0.43 | 6.79 | 7.22 | 157.34 | 4.32 | B |
| SER | 308 | 2.56 | 0.03 | 2.59 | 104.76 | 0.02 | B |
| GLY | 309 | 0.06 | 0.00 | 0.06 | 30.80 | 0.00 | B |
| TRP | 310 | 0.48 | 6.95 | 7.43 | 267.74 | 2.60 | B |
| GLY | 311 | 14.74 | 1.12 | 15.86 | 30.80 | 3.63 | B |
| ARG | 312 | 27.44 | 38.98 | 66.42 | 249.57 | 15.62 | B |
| VAL | 313 | 39.36 | 64.84 | 104.20 | 157.34 | 41.21 | E |
| PHE | 314 | 16.57 | 135.17 | 151.73 | 220.07 | 61.42 | E |
| LYS | 316 | 29.68 | 122.27 | 151.95 | 217.63 | 56.18 | E |
| GLY | 317 | 11.82 | 0.00 | 11.82 | 30.80 | 0.00 | B |
| ARG | 318 | 7.84 | 121.48 | 129.32 | 249.57 | 48.67 | E |
| SER | 319 | 3.30 | 5.88 | 9.18 | 104.76 | 5.61 | B |
| ALA | 320 | 14.51 | 29.06 | 43.57 | 86.68 | 33.52 | E |
| LEU | 321 | 0.05 | 87.51 | 87.55 | 185.25 | 47.24 | E |
| VAL | 322 | 0.00 | 20.76 | 20.76 | 157.34 | 13.19 | B |
| LEU | 323 | 0.00 | 6.41 | 6.41 | 185.25 | 3.46 | B |
| GLN | 324 | 2.61 | 21.41 | 24.02 | 183.52 | 11.66 | B |
| TYR | 325 | 2.73 | 19.35 | 22.09 | 232.25 | 8.33 | B |
| LEU | 326 | 0.00 | 0.03 | 0.03 | 185.25 | 0.02 | B |
| ARG | 327 | 0.00 | 113.77 | 113.77 | 249.57 | 45.59 | E |
| VAL | 328 | 0.01 | 1.68 | 1.69 | 157.34 | 1.07 | B |
| PRO | 329 | 0.02 | 39.18 | 39.20 | 158.05 | 24.79 | B |
| LEU | 330 | 17.34 | 52.97 | 70.31 | 185.25 | 28.59 | E |
| VAL | 331 | 5.59 | 3.04 | 8.63 | 157.34 | 1.93 | B |
| ASP | 332 | 18.64 | 75.55 | 94.18 | 135.82 | 55.62 | E |
| ARG | 333 | 9.89 | 133.09 | 142.98 | 249.57 | 53.33 | E |
| ALA | 334 | 8.14 | 30.31 | 38.44 | 86.68 | 34.96 | E |
| THR | 335 | 2.05 | 47.23 | 49.27 | 141.80 | 33.31 | E |
| CYS | 336 | 0.00 | 0.22 | 0.22 | 129.22 | 0.17 | B |
| LEU | 337 | 2.01 | 79.09 | 81.10 | 185.25 | 42.69 | E |
| ARG | 338 | 33.37 | 177.73 | 211.10 | 249.57 | 71.21 | E |
| SER | 339 | 20.57 | 5.99 | 26.56 | 104.76 | 5.72 | B |
| THR | 340 | 20.65 | 4.56 | 25.21 | 141.80 | 3.22 | B |
| LYS | 341 | 43.24 | 145.22 | 188.46 | 217.63 | 66.73 | E |
| PHE | 342 | 14.62 | 24.42 | 39.04 | 220.07 | 11.10 | B |
| THR | 343 | 9.93 | 93.74 | 103.68 | 141.80 | 66.11 | E |
| ILE | 344 | 19.59 | 6.66 | 26.25 | 187.72 | 3.55 | B |
| TYR | 345 | 9.63 | 83.94 | 93.56 | 232.25 | 36.14 | E |
| ASN | 346 | 8.78 | 116.51 | 125.29 | 152.56 | 76.37 | E |
| ASN | 347 | 1.44 | 17.46 | 18.90 | 152.56 | 11.44 | B |
| MET | 348 | 5.65 | 1.01 | 6.66 | 195.71 | 0.52 | B |
| PHE | 349 | 2.97 | 5.49 | 8.46 | 220.07 | 2.50 | B |
| CYS | 350 | 0.34 | 0.00 | 0.34 | 129.22 | 0.00 | B |
| ALA | 351 | 2.12 | 0.15 | 2.27 | 86.68 | 0.17 | B |
| GLY | 352 | 2.51 | 0.00 | 2.51 | 30.80 | 0.00 | B |
| PHE | 353 | 1.64 | 19.67 | 21.31 | 220.07 | 8.94 | B |
| GLU | 355 | 13.85 | 62.67 | 76.51 | 168.29 | 37.24 | E |
| GLY | 356 | 20.68 | 0.00 | 20.69 | 30.80 | 0.01 | B |
| GLY | 357 | 22.76 | 4.81 | 27.58 | 30.80 | 15.63 | B |
| ARG | 358 | 1.89 | 66.71 | 68.60 | 249.57 | 26.73 | E |
| ASP | 359 | 9.52 | 11.56 | 21.07 | 135.82 | 8.51 | B |
| SER | 360 | 13.75 | 11.98 | 25.73 | 104.76 | 11.43 | B |
| CYS | 361 | 14.62 | 38.52 | 53.15 | 129.22 | 29.81 | E |
| GLN | 362 | 1.88 | 39.36 | 41.24 | 183.52 | 21.45 | B |
| GLY | 363 | 12.98 | 1.09 | 14.07 | 30.80 | 3.53 | B |
| ASP | 364 | 3.70 | 4.29 | 7.99 | 135.82 | 3.16 | B |
| SER | 365 | 1.74 | 5.22 | 6.96 | 104.76 | 4.98 | B |
| GLY | 366 | 0.00 | 0.00 | 0.00 | 30.80 | 0.00 | B |
| GLY | 367 | 2.61 | 1.56 | 4.17 | 30.80 | 5.07 | B |
| PRO | 368 | 0.92 | 0.00 | 0.92 | 158.05 | 0.00 | B |
| VAL | 370 | 0.00 | 1.87 | 1.87 | 157.34 | 1.19 | B |
| THR | 371 | 0.36 | 6.97 | 7.34 | 141.80 | 4.92 | B |
| GLU | 372 | 30.42 | 54.74 | 85.16 | 168.29 | 32.53 | E |
| VAL | 373 | 4.82 | 14.38 | 19.20 | 157.34 | 9.14 | B |
| GLU | 374 | 42.40 | 67.06 | 109.46 | 168.29 | 39.85 | E |
| GLY | 375 | 14.57 | 17.57 | 32.14 | 30.80 | 57.04 | E |
| THR | 376 | 0.00 | 0.00 | 0.00 | 141.80 | 0.00 | B |
| SER | 377 | 0.00 | 9.63 | 9.63 | 104.76 | 9.19 | B |
| PHE | 378 | 0.00 | 0.08 | 0.08 | 220.07 | 0.04 | B |
| LEU | 379 | 0.31 | 0.02 | 0.33 | 185.25 | 0.01 | B |
| THR | 380 | 0.00 | 0.00 | 0.00 | 141.80 | 0.00 | B |
| GLY | 381 | 0.00 | 0.00 | 0.00 | 30.80 | 0.00 | B |
| ILE | 382 | 0.00 | 0.00 | 0.00 | 187.72 | 0.00 | B |
| ILE | 383 | 0.00 | 2.10 | 2.10 | 187.72 | 1.12 | B |
| SER | 384 | 1.95 | 0.00 | 1.95 | 104.76 | 0.00 | B |
| TRP | 385 | 1.30 | 110.40 | 111.70 | 267.74 | 41.24 | E |
| GLY | 386 | 23.92 | 0.65 | 24.57 | 30.80 | 2.12 | B |
| GLU | 387 | 32.54 | 33.31 | 65.85 | 168.29 | 19.80 | B |
| GLU | 388 | 9.64 | 90.20 | 99.84 | 168.29 | 53.60 | E |
| CYS | 389 | 31.77 | 22.87 | 54.64 | 129.22 | 17.70 | B |
| ALA | 390 | 25.02 | 65.91 | 90.92 | 86.68 | 76.04 | E |
| MET | 391 | 10.13 | 86.20 | 96.33 | 195.71 | 44.04 | E |
| LYS | 392 | 18.83 | 160.20 | 179.02 | 217.63 | 73.61 | E |
| GLY | 393 | 46.51 | 15.41 | 61.92 | 30.80 | 50.03 | E |
| LYS | 394 | 5.28 | 37.72 | 43.00 | 217.63 | 17.33 | B |
| TYR | 395 | 15.71 | 24.41 | 40.12 | 232.25 | 10.51 | B |
| GLY | 396 | 6.12 | 2.98 | 9.09 | 30.80 | 9.66 | B |
| ILE | 397 | 0.00 | 3.32 | 3.32 | 187.72 | 1.77 | B |
| TYR | 398 | 0.00 | 6.22 | 6.22 | 232.25 | 2.68 | B |
| THR | 399 | 0.00 | 0.00 | 0.00 | 141.80 | 0.00 | B |
| LYS | 400 | 0.00 | 32.89 | 32.89 | 217.63 | 15.11 | B |
| VAL | 401 | 0.00 | 0.00 | 0.00 | 157.34 | 0.00 | B |
| SER | 402 | 0.00 | 10.50 | 10.50 | 104.76 | 10.03 | B |
| ARG | 403 | 16.40 | 77.09 | 93.49 | 249.57 | 30.89 | E |
| TYR | 404 | 0.00 | 0.94 | 0.94 | 232.25 | 0.41 | B |
| VAL | 405 | 0.82 | 0.86 | 1.68 | 157.34 | 0.55 | B |
| ASN | 406 | 1.90 | 94.81 | 96.71 | 152.56 | 62.15 | E |
| TRP | 407 | 3.71 | 52.87 | 56.58 | 267.74 | 19.75 | B |
| ILE | 408 | 0.00 | 0.08 | 0.08 | 187.72 | 0.04 | B |
| LYS | 409 | 1.34 | 104.52 | 105.87 | 217.63 | 48.03 | E |
| GLU | 410 | 4.61 | 86.16 | 90.77 | 168.29 | 51.20 | E |
| LYS | 411 | 5.92 | 54.20 | 60.12 | 217.63 | 24.90 | B |
| THR | 412 | 4.57 | 11.00 | 15.57 | 141.80 | 7.75 | B |
| LYS | 413 | 18.60 | 112.34 | 130.93 | 217.63 | 51.62 | E |
| LEU | 414 | 33.54 | 129.02 | 162.55 | 185.25 | 69.65 | E |
| THR | 415 | 42.16 | 44.61 | 86.77 | 141.80 | 31.46 | E |

Total area: 26818.00

1. An isolated Factor IX (FIX) protein variant comprising one or more than one additional glycosylation site as compared to wild type Factor IX.

2. The FIX variant of claim 1, wherein at least one of the one or more than one additional glycosylation sites is in the activation peptide.

3. The FIX variant of claim 1, comprising a peptide segment inserted between position N157 and N167 of the human FIX amino acid sequence of SEQ ID NO:33.

4. The FIX variant of claim 3, wherein the peptide segment comprises 3-100 amino acid residues.

5. The FIX variant of claim 4, wherein the peptide segment comprises at least part of a mouse Factor IX activation peptide.

6. The FIX variant of claim 5, wherein the mouse activation peptide is modified to increase the number of glycosylation sites.

7. The FIX variant of claim 1, wherein the one or more than one additional glycosylation sites are selected from N-linked glycosylation site(s), O-linked glycosylation site(s) and a combination of N-linked glycosylation site(s) and O-linked glycosylation site(s).

8. The FIX variant of claim 7, wherein the glycosylation site(s) comprise N-linked glycosylation site(s) comprising a consensus sequence NXT/S, with the proviso that X is not proline.

9. The FIX variant of claim 7, wherein the glycosylation site(s) comprise O-linked glycosylation site(s) comprising a consensus sequence selected from the group consisting of CXXGGT/S-C (SEQ ID NO:9), NSTE/DA (SEQ ID NO:10), NITQS (SEQ ID NO:11), QSTQS (SEQ ID NO:12), D/E-FT-R/K-V (SEQ ID NO:13), C-E/D-SN (SEQ ID NO:14), GGSC-K/R (SEQ ID NO:15) and any combination thereof.

10. The FIX variant of claim 1, comprising 1-5 additional glycosylation sites.

11. A vector comprising a nucleotide sequence encoding the FIX variant of claim 1.

12. A transformed cell comprising the vector of claim 11.

13. A transgenic animal comprising the FIX variant of claim 1.

14. The FIX variant of claim 1, wherein at least one additional glycosylation site is outside of the activation peptide.

15. The FIX variant of claim 14, wherein the at least one additional glycosylation site corresponds to a site that is glycosylated in the native form of a non-human homolog of FIX.

16. The FIX variant of claim 15, wherein the non-human homolog is selected from the group consisting of dog, pig, cow, and mouse.

17. A method of increasing the number of glycosylation sites in a Factor IX protein comprising:
a) aligning a first FIX amino acid sequence and a second FIX amino acid sequence;
b) identifying a glycosylation site in the first FIX amino acid sequence that is not present in the second FIX amino acid sequence; and
c) modifying the second FIX amino acid sequence to introduce a glycosylation site corresponding to the glycosylation site identified in the first FIX amino acid sequence of step (b),
wherein modifying the second FIX amino acid sequence increases the number of glycosylation sites in the FIX protein.

18. The method of claim 17, wherein the first FIX amino acid sequence is from a non-human species and the second FIX amino acid sequence is human FIX.

19. The method of claim 17, wherein the glycosylation site in the first FIX amino acid sequence is in the activation peptide.

20. The method of claim 17, wherein the glycosylation site in the first FIX amino acid sequence is outside of the activation peptide.

21. The FIX variant of claim 1, which is a human FIX protein.

22. The FIX variant of claim 1, wherein the one or more additional glycosylation sites are introduced by insertion of additional amino acids, deletion of amino acids, substitution of amino acids and/or rearrangement of amino acids, in any combination.

23. The FIX variant of claim 1, wherein the one or more additional glycosylation sites are introduced by site-directed mutagenesis.

24. The FIX variant of claim 1, wherein the one or more additional glycosylation sites are introduced by chemical synthesis of the FIX variant.

25. An isolated FIX variant comprising one or more additional sugar chains as compared to wild type FIX.

26. The isolated FIX variant of claim 25, wherein said one or more additional sugar chains are added to the FIX protein by chemical and/or enzymatic methods.
